(19) United States
(12) Patent Application Publication
(10) Pub. No.: US 2024/0040336 A1
(43) Pub. Date: Feb. 1, 2024

(54) Managing Off-Earth and Specialty Community Agriculture

(71) Applicant: Conquer Your Addiction LLC, Kirkwood, MO (US)

(72) Inventors: David H. WILLIAMS, Kirkwood, MO (US); Adam H. WILLIAMS, Kirkwood, MO (US)

(21) Appl. No.: 18/372,544

(22) Filed: Sep. 25, 2023

Related U.S. Application Data
(63) Continuation-in-part of application No. 18/211,999, filed on Jun. 20, 2023.
Application No. 18/211,999 is a continuation-in-part of application No. 18/090,047, filed on Dec. 28, 2022.
Application No. 18/090,047 is a continuation-in-part of application No. 17/903,419, filed on Sep. 6, 2022.
Application No. 17/903,419 is a continuation-in-part of application No. 17/882,061, filed on Aug. 5, 2022.
Application No. 17/882,061 is a continuation-in-part of application No. 17/861,559, filed on Jul. 11, 2022.
Application No. 17/861,559 is a continuation-in-part of application No. 17/541,707, filed on Dec. 3, 2021.
Application No. 17/541,707 is a continuation-in-part of application No. 18/090,047, filed on Dec. 28, 2022.
Application No. 18/090,047 is a continuation-in-part of application No. 17/192,381, filed on Mar. 4, 2021, now Pat. No. 11,636,941.
Application No. 17/192,381 is a continuation-in-part of application No. 17/861,559, filed on Jul. 11, 2022.
Application No. 17/861,559 is a continuation-in-part of application No. 17/882,061, filed on Aug. 5, 2022.
Application No. 17/882,061 is a continuation-in-part of application No. 17/541,707, filed on Dec. 3, 2021.
Application No. 17/541,707 is a continuation-in-part of application No. 17/903,419, filed on Sep. 6, 2022.
Application No. 17/861,559 is a continuation-in-part of application No. 16/700,601, filed on Dec. 2, 2019, now Pat. No. 11,388,546.
Application No. 17/882,061 is a continuation of application No. 17/104,136, filed on Nov. 25, 2020, now Pat. No. 11,412,353.
Application No. 17/104,136 is a continuation-in-part of application No. 16/700,601, filed on Dec. 2, 2019, now Pat. No. 11,388,546.
Application No. 16/700,601 is a continuation-in-part of application No. 17/104,136, filed on Nov. 25, 2020, now Pat. No. 11,412,353.
Application No. 17/104,136 is a continuation-in-part of application No. 17/192,381, filed on Mar. 4, 2021, now Pat. No. 11,636,941.
Application No. 17/192,381 is a continuation-in-part of application No. 17/861,559, filed on Jul. 11, 2022.
Application No. 17/861,559 is a continuation-in-part of application No. 17/882,061, filed on Aug. 5, 2022.
Application No. 17/882,061 is a continuation-in-part of application No. 17/192,381, filed on Mar. 4, 2021, now Pat. No. 11,636,941.
Application No. 17/192,381 is a continuation-in-part of application No. 17/541,707, filed on Dec. 3, 2021.
Application No. 17/541,707 is a continuation-in-part of application No. 16/654,708, filed on Oct. 16, 2019, now Pat. No. 10,853,897.
Application No. 16/654,708 is a continuation-in-part of application No. 62/746,330, filed on Oct. 16, 2018.
Application No. 62/746,330 is a continuation-in-part of application No. 16/516,822, filed on Jul. 19, 2019, now Pat. No. 10,497,242.
Application No. 16/516,822 is a continuation-in-part of application No. 15/840,762, filed on Dec. 13, 2017, now Pat. No. 10,477,342.
Application No. 15/840,762 is a continuation-in-part of application No. 15/840,762, filed on Dec. 13, 2017, now Pat. No. 10,477,342.
Application No. 16/700,601 is a continuation of application No. 15/840,775, filed on Dec. 13, 2017, now Pat. No. 10,555,112.
(60) Provisional application No. 63/460,523, filed on Apr. 19, 2023.
(60) Provisional application No. 63/441,569, filed on Jan. 27, 2023.
(60) Provisional application No. 63/460,523, filed on Apr. 19, 2023.
(60) Provisional application No. 63/441,569, filed on Jan. 27, 2023.
(60) Provisional application No. 63/344,976, filed on May 23, 2022.
(60) Provisional application No. 63/294,815, filed on Dec. 29, 2021.
(60) Provisional application No. 63/344,976, filed on May 23, 2022.
(60) Provisional application No. 63/316,277, filed on Mar. 3, 2022.
(60) Provisional application No. 63/120,834, filed on Dec. 3, 2020.
(60) Provisional application No. 63/344,976, filed on May 23, 2022.
(60) Provisional application No. 63/316,277, filed on Mar. 3, 2022.
(60) Provisional application No. 63/294,815, filed on Dec. 29, 2021.
(60) Provisional application No. 63/275,300, filed on Nov. 3, 2021.
(60) Provisional application No. 63/011,949, filed on Apr. 17, 2020.
(60) Provisional application No. 62/701,252, filed on Jul. 20, 2018.
(60) Provisional application No. 62/435,042, filed on Dec. 15, 2016.
(60) Provisional application No. 62/480,206, filed on Mar. 31, 2017.

(51) Int. Cl. H04W 4/029; G06Q 50/26; A61B 5/16; H04W 4/021

(52) U.S. Cl. CPC H04W 4/029; G06Q 50/265; A61B 5/165; H04W 4/021

(57) ABSTRACT

Exemplary embodiments are disclosed of systems, methods, and technologies for managing off-Earth and specialty community agriculture. In exemplary embodiments, a system is configured for managing specialty community agriculture and associated resources utilizing needs-based, context-based, and behavior-driven integrated production and consumption resource management capabilities and resource inputs. The system comprises a plurality of different devices, sensors, other systems, and/or communications network(s) configured to dynamically and flexibly manage modular planting, growing, treatment, harvesting, producing, acquiring, generating, distribution, storage, and/or consumption of consumable resources of a community(s). The system is configured to be operable for determining, assessing, analyzing, (re)allocating, and/or predicting future production and/or consumption resources to support future forecasted, predicted, and/or possible needs and associated behaviors and contexts of at least a plurality of humans within, expected to be within, and/or requirement to be within a human community population of the community(s).

### CROSS-REFERENCE TO RELATED APPLICATIONS

The present application claims the benefit and priority of U.S. Provisional Patent Application No. 63/460,523 filed Apr. 19, 2023.

The present application claims the benefit and priority of U.S. Provisional Patent Application No. 63/441,569 filed Jan. 27, 2023.

The present application is a continuation-in-part of U.S. patent application Ser. No. 18/211,999 filed Jun. 20, 2023.

The present application is a continuation-in-part of U.S. patent application Ser. No. 18/090,047 filed Dec. 28, 2022, which published as US2023/0179955 on Jun. 8, 2023.

The present application is a continuation-in-part of U.S. patent application Ser. No. 17/903,419 filed Sep. 6, 2022, which published as US2023/0007439 on Jan. 5, 2023.

The present application is a continuation-in-part of U.S. patent application Ser. No. 17/882,061 filed Aug. 5, 2022, which published as US2022/0386080 on Dec. 1, 2022.

The present application is a continuation-in-part of U.S. patent application Ser. No. 17/861,559 filed Jul. 11, 2022, which published as US2022/0353632 on Nov. 3, 2022.

The present application is a continuation-in-part of U.S. patent application Ser. No. 17/541,707 filed Dec. 3, 2021, which published as US2022/0116736.

U.S. patent application Ser. No. 18/211,999 claims the benefit and priority of (1) U.S. Provisional Patent Application No. 63/460,523 filed Apr. 19, 2023; (2) U.S. Provisional Patent Application No. 63/441,569 filed Jan. 27, 2023; and (3) U.S. Provisional Patent Application No. 63/344,976 filed May 23, 2022. U.S. patent application Ser. No. 18/211,999 is a continuation-in-part of U.S. patent application Ser. No. 18/090,047 filed Dec. 28, 2022.

U.S. patent application Ser. No. 18/090,047 is a continuation-in-part of U.S. patent application Ser. No. 17/192,381 filed Mar. 4, 2021, which published as US2021/0202067 on Jul. 1, 2021.

U.S. patent application Ser. No. 18/090,047 claims the benefit and priority of (1) U.S. Provisional Patent Application No. 63/294,815 filed Dec. 29, 2021; (2) U.S. Provisional Patent Application No. 63/344,976 filed May 23, 2022; and (3) U.S. Provisional Patent Application No. 63/316,277 filed Mar. 2, 2022.

U.S. patent application Ser. No. 18/090,047 is also a continuation-in-part of (1) U.S. patent application Ser. No. 17/861,559 filed Jul. 11, 2022, which published as US2022/0353632 on Nov. 3, 2022; (2) U.S. patent application Ser. No. 17/882,061 filed Aug. 5, 2022, which published as US2022/0386080 on Dec. 1, 2022; (3) U.S. patent application Ser. No. 17/541,707 filed Dec. 3, 2021, which published as US2022/0116736 on Apr. 14, 2022; and (4) U.S. patent application Ser. No. 17/903,419 filed Sep. 6, 2022.

U.S. patent application Ser. No. 17/192,381 claims the benefit and priority of (1) U.S. Provisional Patent Application No. 62/986,382 filed Mar. 6, 2020; and (2) U.S. Provisional Patent Application No. 63/011,949 filed Apr. 17, 2020. U.S. patent application Ser. No. 17/192,381 is a continuation-in-part of U.S. patent application Ser. No. 17/104,136.

U.S. patent application Ser. No. 17/861,559 is a continuation-in-part of U.S. patent application Ser. No. 16/700,601 filed Dec. 2, 2019, which published as US2020/0107155 on Apr. 2, 2020 and issued as U.S. Pat. No. 11,388,546 on Jul. 12, 2022.

U.S. patent application Ser. No. 17/882,061 is a continuation of U.S. patent application Ser. No. 17/104,136 filed Nov. 25, 2020, which published as US2020/0084451 on Mar. 18, 2021 and issued as U.S. Pat. No. 11,412,353 on Aug. 9, 2022.

U.S. patent application Ser. No. 17/541,707 claims the benefit and priority of U.S. Provisional Patent Application No. 63/120,834 filed Dec. 3, 2020. U.S. patent application Ser. No. 17/541,707 is a continuation-in-part of (1) U.S. patent application Ser. No. 16/700,601; (2) U.S. patent application Ser. No. 17/104,136; and (3) U.S. patent application Ser. No. 17/192,381.

U.S. patent application Ser. No. 17/903,419 claims the benefit and priority of (1) U.S. Provisional Patent Application No. 63/344,976 filed May 23, 2022; (2) U.S. Provisional Patent Application No. 63/316,277 filed Mar. 2, 2022; (3) U.S. Provisional Patent Application No. 63/294,815 filed Dec. 29, 2021; and (4) U.S. Provisional Patent Application No. 63/275,300 filed Nov. 3, 2021.

U.S. patent application Ser. No. 17/903,419 is also a continuation-in-part of (1) U.S. patent application Ser. No. 17/861,559 filed Jul. 11, 2022, which published as US2022/0353632 on Nov. 3, 2022; (2) U.S. patent application Ser. No. 17/882,061 filed Aug. 5, 2022, which published as US2022/0386080 on Dec. 1, 2022; (3) U.S. patent application Ser. No. 17/192,381 filed Mar. 4, 2021, which published as US2021/0202067 on Jul. 1, 2021; and (4) U.S. patent application Ser. No. 17/541,707 filed Dec. 3, 2021, which published as US2022/0116736 on Apr. 14, 2022.

U.S. patent application Ser. No. 17/104,136 claims the benefit and priority of U.S. Provisional Patent Application No. 63/011,949 filed Apr. 17, 2020. U.S. patent application Ser. No. 17/104,136 is a continuation-in-part of U.S. patent application Ser. No. 16/654,708 filed Oct. 16, 2019, which published as US2020/0051189 on Feb. 13, 2020 and issued as U.S. Pat. No. 10,853,897 on Dec. 1, 2020.

U.S. patent application Ser. No. 16/654,708 claims the benefit and priority of U.S. Provisional Patent Application No. 62/746,330 filed Oct. 16, 2018. U.S. patent application Ser. No. 16/654,708 is a continuation-in-part of U.S. patent application Ser. No. 16/516,822 filed Jul. 19, 2019, which published as US2019/0340906 on Nov. 7, 2019 and issued as U.S. Pat. No. 10,497,242 on Dec. 3, 2019. U.S. patent application Ser. No. 16/654,708 is also a continuation-in-part of U.S. patent application Ser. No. 15/840,762 filed Dec. 13, 2017, which published as US2018/0176727 on Jun. 21, 2018 and issued as U.S. Pat. No. 10,477,342 on Nov. 12, 2019.

U.S. patent application Ser. No. 16/516,822 claims the benefit and priority of U.S. Provisional Patent Application No. 62/701,252 filed Jul. 20, 2018. U.S. patent application Ser. No. 16/516,822 is a continuation-in-part of U.S. patent application Ser. No. 15/840,762.

U.S. patent application Ser. No. 16/700,601 is continuation of U.S. patent application Ser. No. 15/840,775 filed Dec. 13, 2017, which published as US2018/0173866 on Jun. 21, 2018 and issued as U.S. Pat. No. 10,555,112 on Feb. 4, 2020.

U.S. patent application Ser. No. 15/840,775 claims the benefit and priority of (1) U.S. Provisional Patent Application No. 62/435,042 filed Dec. 15, 2016; and (2) U.S. Provisional Patent Application No. 62/480,206 filed Mar. 31, 2017.

The entire disclosures of the above patent applications are incorporated herein by reference.

### FIELD

The present disclosure relates to systems, methods, and technologies for managing off-Earth and specialty community agriculture.

### BACKGROUND

This section provides background information related to the present disclosure which is not necessarily prior art.

Specialty community agriculture may include unusual and/or scarce (“scarce”) conditions for growing, production, storage/distribution, and/or consumption. Examples of such specialty community agriculture include specialty greenhouses and high/low depth farming, etc. It also includes the acquisition and/or production of resources included in such agricultural processes and activities, including mining and farming labor, equipment, and associated/related resources.

### DRAWINGS

The drawings described herein are for illustrative purposes only of selected embodiments and not all possible implementations and are not intended to limit the scope of the present disclosure. For example, drawings may be described herein with reference to an addict but in other exemplary embodiments the systems and devices shown in the drawings may be modified or configured to be usable for managing off-Earth and specialty community agriculture as disclosed herein.

FIG. 1 is a diagram of an example system for determining location and context of an addict, a support network, and other information and aspects of an addict's personal and professional life for addiction treatment purposes, including relapse prevention and containment. This diagram includes various example networks and technologies that may be used for collecting and analyzing the addict's location and context. Also shown are example data sources and analytical engines that may be needed to process such data and to identify and implement actions to preempt, prevent, and/or contain any relapse. In other exemplary embodiments, the example system shown in FIG. 1 (and other figures) may be modified or configured to be usable for determining location and context of an asset or entity of a community (e.g., person, robot, etc.) when managing off-Earth and specialty community agriculture as disclosed herein.

FIG. 2 describes an example Addict Monitor/Controller (AMC) device that may be used to collect, process, and disseminate context and addiction trigger-related data from and about an addict via various sensors and other data collection mechanisms, and to interface with/to the addict and 3rd party mechanisms. The device may also provide mechanisms to provide feedback to the addict and assist in the implementation of relapse-related preventative and containment actions. In other exemplary embodiments, the AMC device shown in FIG. 2 may be modified or configured to be usable to collect, process, and disseminate context and trigger-related data from and about an asset or entity of a community when managing off-Earth and specialty community agriculture as disclosed herein.

FIG. 2a provides examples of distributed sensor deployment, data collection options, localized sensors, and localized networks that may be used in exemplary embodiments.

FIG. 2b provides examples of internet of things (IoT) addict-related sensors, devices, and networks that may be used in exemplary embodiments.

FIG. 3 depicts example steps for monitoring an addict's triggers, and in the course of doing so assessing/predicting the addict's risk of relapse. FIG. 3 also describes identifying possible resources that could help the addict, and the actions that could be taken to prevent, preempt or contain a relapse. FIG. 3 also describes an example process for selecting such resources and actions.

FIG. 4 depicts an example system and example process for determining the location/context of an addict as well as the location/context of support resources using a variety of sensors and other information sources.

FIG. 5 describes an example system and example process for assessing an addict's trigger/relapse risk. FIG. 5 also describes how such algorithms could be made self-learning to better assess an addict's relapse risk.

FIG. 5A depicts an example embodiment of a method for managing damage control and recovering from a relapse situation.

FIG. 5B provides examples of risk, support areas maps, and map mashups.

FIG. 6 depicts example ways to identify/monitor trigger/relapse risk and identifying, selecting, and implementing support resources and actions.

FIG. 6A depicts an exemplary embodiment of a trigger monitoring feedback and learning system.

FIG. 7 describes example ways to identify/determine and select the best actions and resources when relapse risk is high.

FIG. 7A describes an example of an action-determining sub process—specifically, ways to utilize regularly scheduled addict community meetings or spontaneous, unscheduled, flash addict community meetings.

FIG. 8 describes example ways to select the best interface(s) for interacting with an addict, including implementing relapse prevention actions.

FIG. 9 describes an example addict rewards/demerits system based on an addict's behaviors and actions, which may include rewarding (or punishing) an addict based on behavior via tracking and data analytics and various reward mechanisms.

FIG. 10 describes example ways in which addicts can receive and transmit sobriety ideas in public and private places via beacons. FIG. 10 also illustrates example ways in which Real-Time Location System (RTLS) technologies can be used to enable ad hoc, spontaneous, unscheduled, or flash addict meetings between people with similar addiction issues.

FIG. 11 thru 14 describe examples of using location and/or context information to provide privacy and security for data collected in various implementations of the present disclosure.

FIG. 15 depicts an example embodiment of a method for monitoring for a risk of a pre-identified behavior (e.g., pre-identified addict-related undesirable behavior, etc.). FIG. 15 also includes example triggers, priorities, and initial risk assessment/detection sensors.

FIG. 16 is a diagram of example conflict battlefield architecture.

FIG. 17 illustrates an example system according to an exemplary embodiment focused on medicine and drug discovery for treatment of disease, including need-based driving, missing data collection, growing extensibility, skill instructional framework, human and AI elements, reputation management with the world, a moral compass/ethical framework to guide, and additional tasks.

FIG. 18 illustrates an example system according to an exemplary embodiment including a moral compass.

FIG. 19 is a diagram depicting various examples of triggers (e.g., drinking triggers, etc.) and how the triggers may be related or interconnected such that one or more trigger(s) may activate one or more related trigger(s).

FIG. 20 is a table illustrating various examples of evidence in the form of blockchain transactions that may be established during the course of production with potential value being added, e.g., using cryptocurrency during key steps of production.

FIG. 21 shows different stages of worms within regolith transformation into soil as an example supply that may be managed according to exemplary embodiments disclosed herein for expanding productive capabilities of a community. In exemplary embodiments, the system is configured to be operable for helping with balancing both supply and demand of a community and also expanding productive capabilities. In exemplary embodiments, the system is configured to be operable for managing transformation of regolith into soils using microbes, earthworms, etc. As recognized herein, regolith transformation into soils requires a proper ratio and environments to allow the worms to thrive within a regolith/manure mixture. During the regolith to soil transformation, pH will need to be managed, more manure may need to be added, trace elements may be necessary, water levels altered, etc. Also, the health of the worms will need to be monitored and improved for worm survivability and increases such that there are as many worms living as possible as part of the larger processing pipeline.

FIG. 22 illustrates a pipeline illustrating a stepwise nature of process and management achievable with exemplary embodiments of the present disclosure. In exemplary embodiments, a system is configured to be operable to monitor current resources, cross reference to the current and future needs of the community including possible additions or removals. This information may then be used to advise or direct the community to expand resource production. In a non-traditional environment such as an Off-Earth community, the system may be configured to advise or direct the expansion of productive capabilities of the community. The expansion of the productive capabilities of the community in Off-Earth will be such that it can advise or direct the transformation of ISRU (in situ resource utilization—aka local resources) into supporting materials for production of bioproducts and support of community health and longevity.

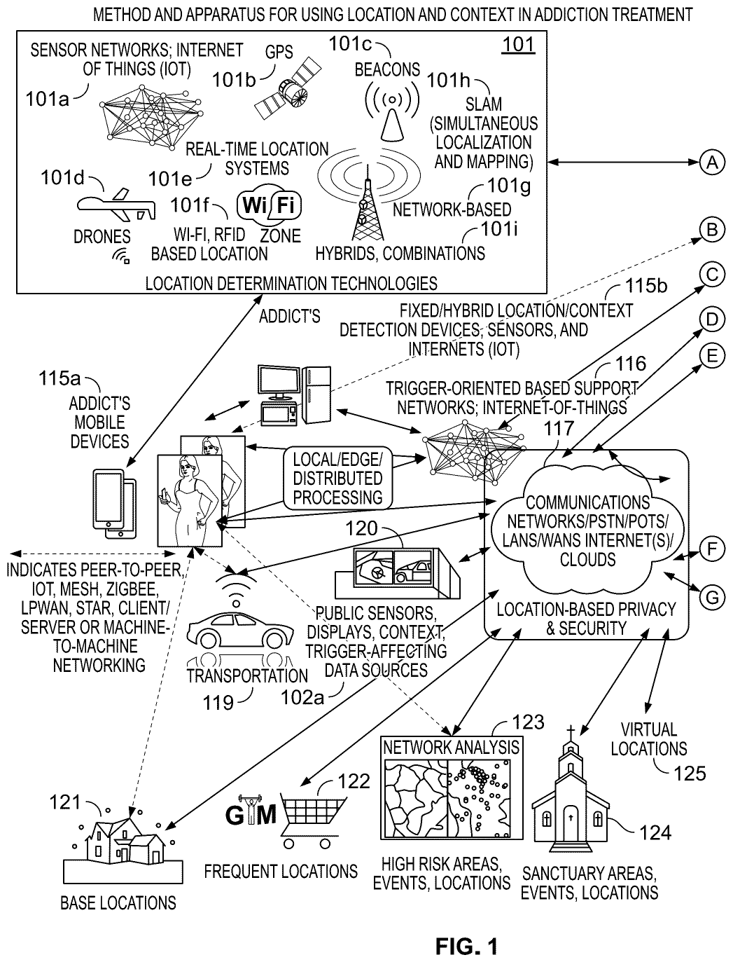

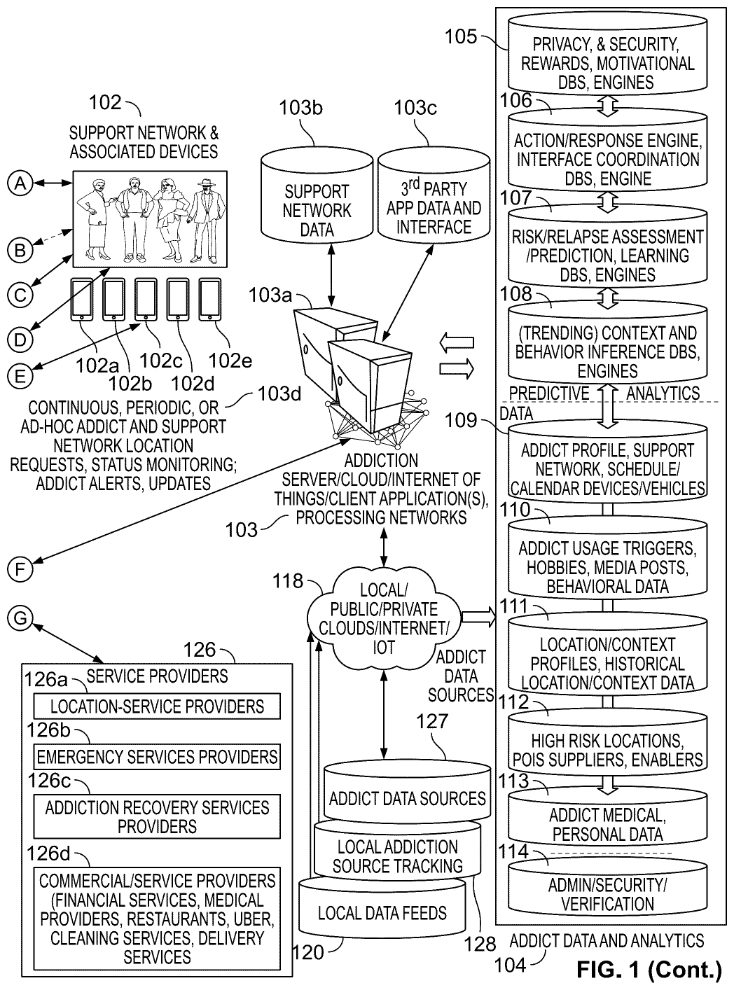

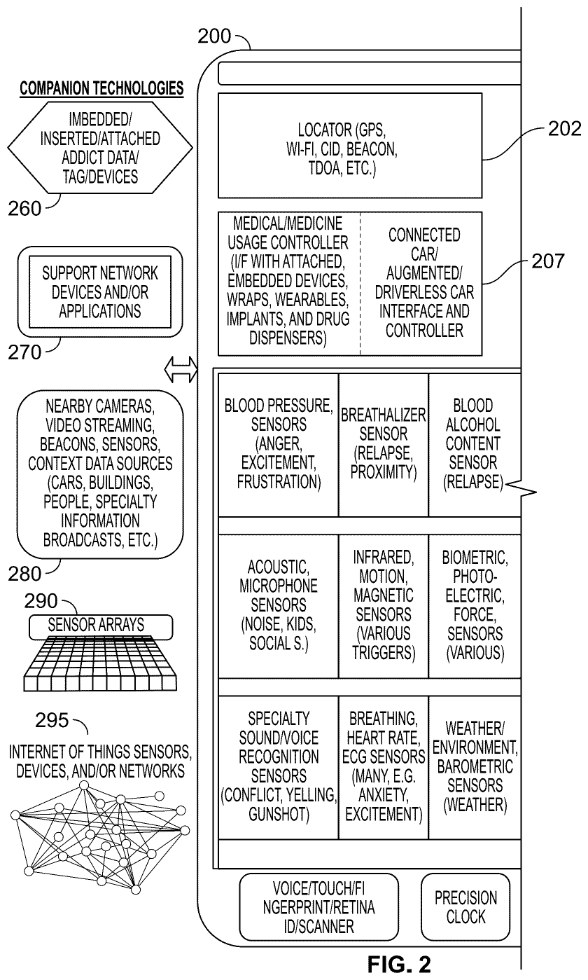

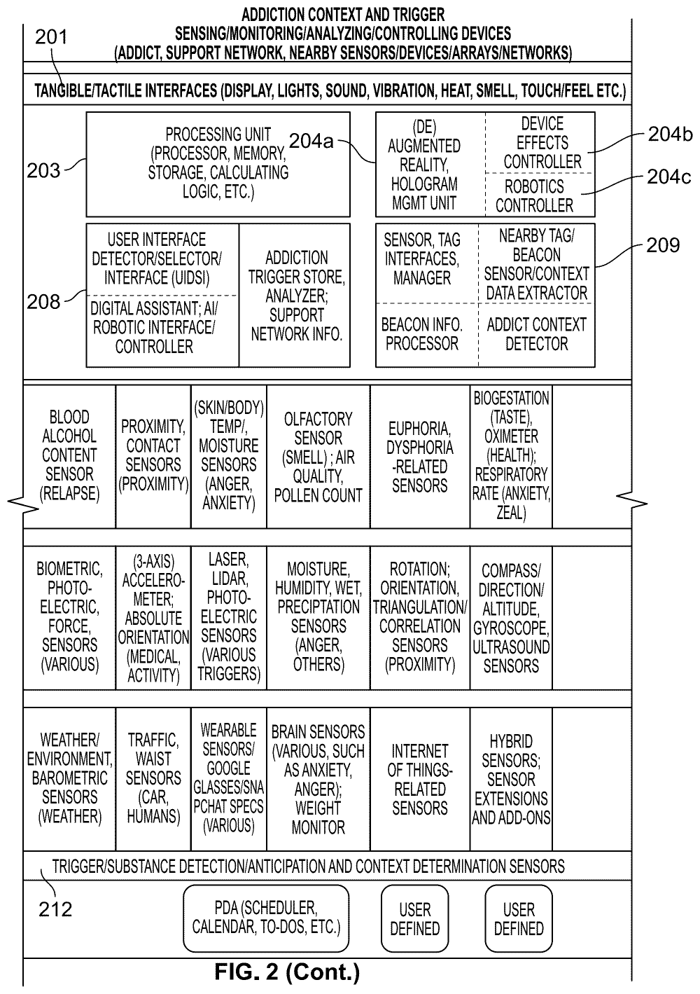

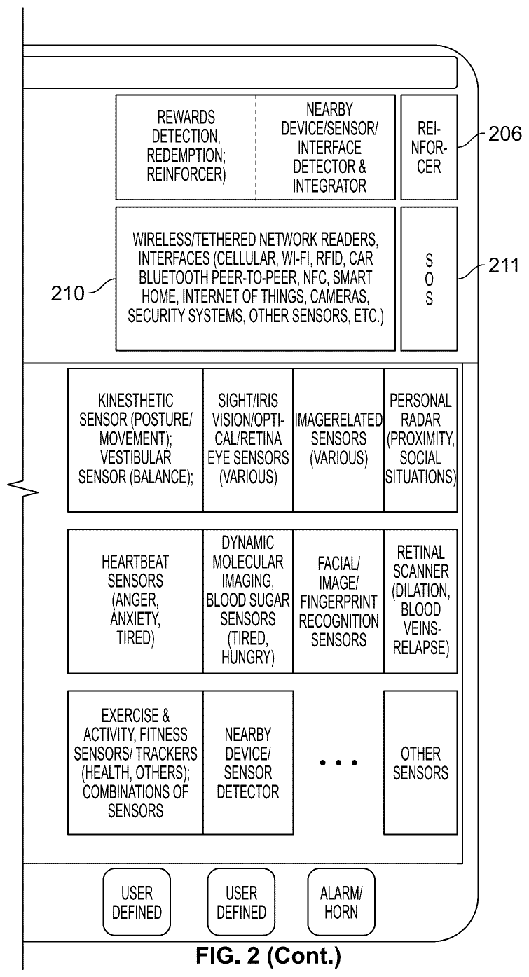

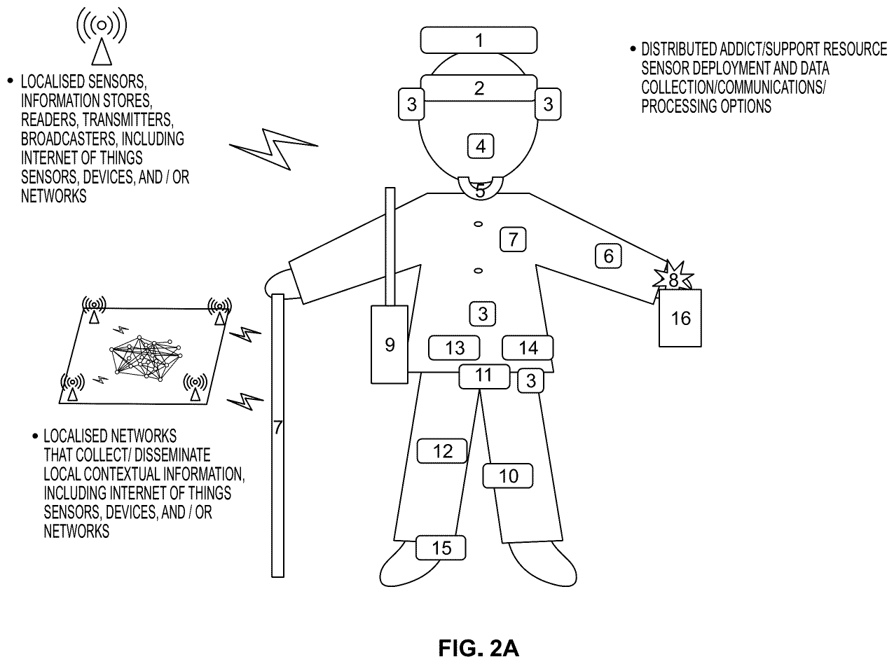

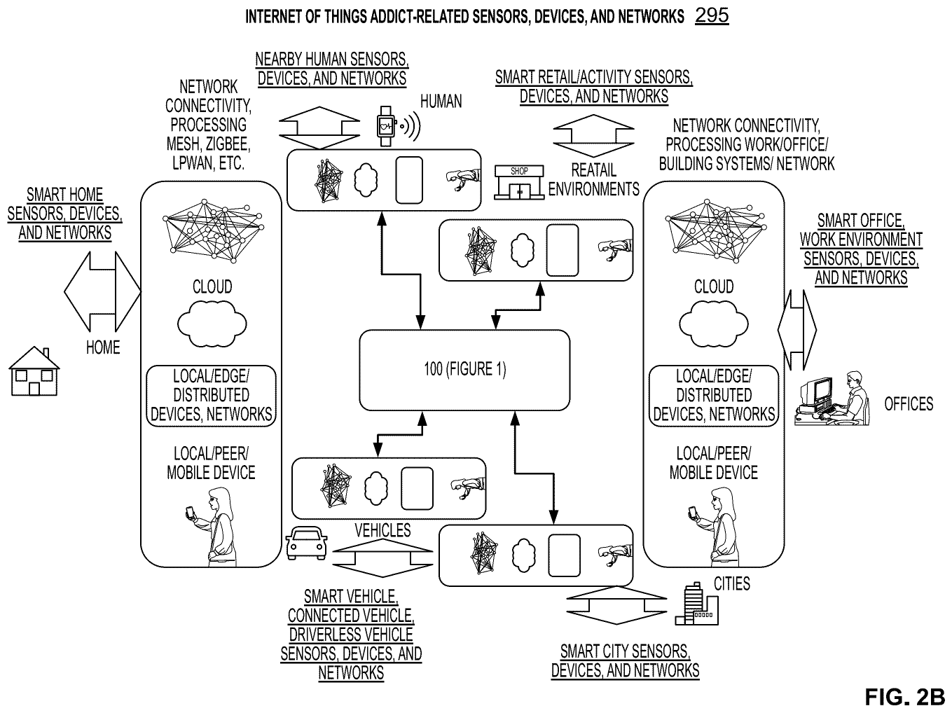

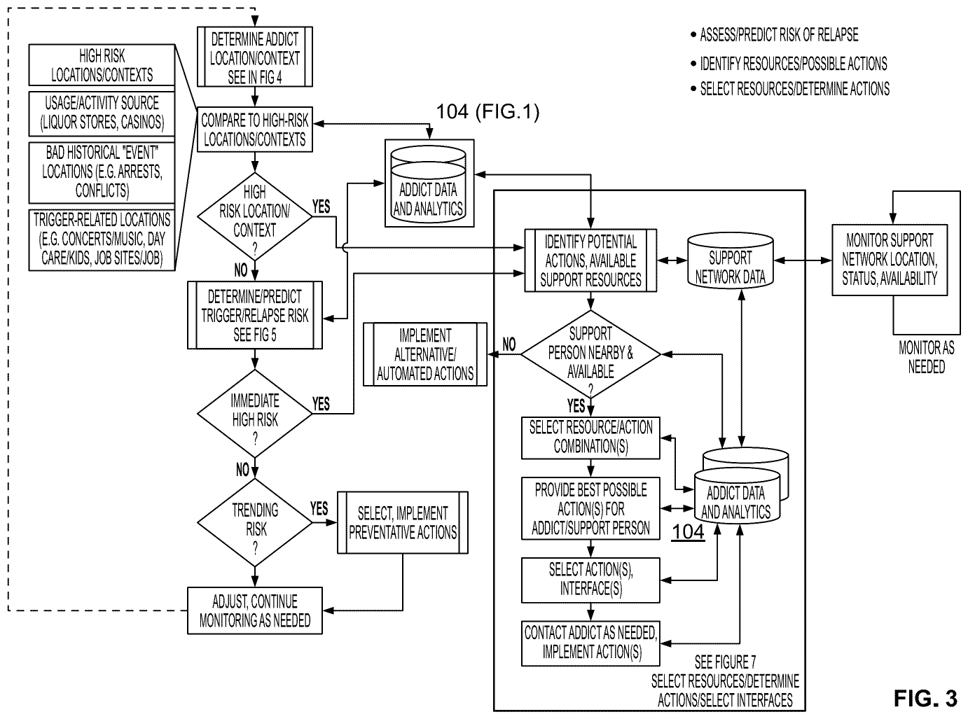

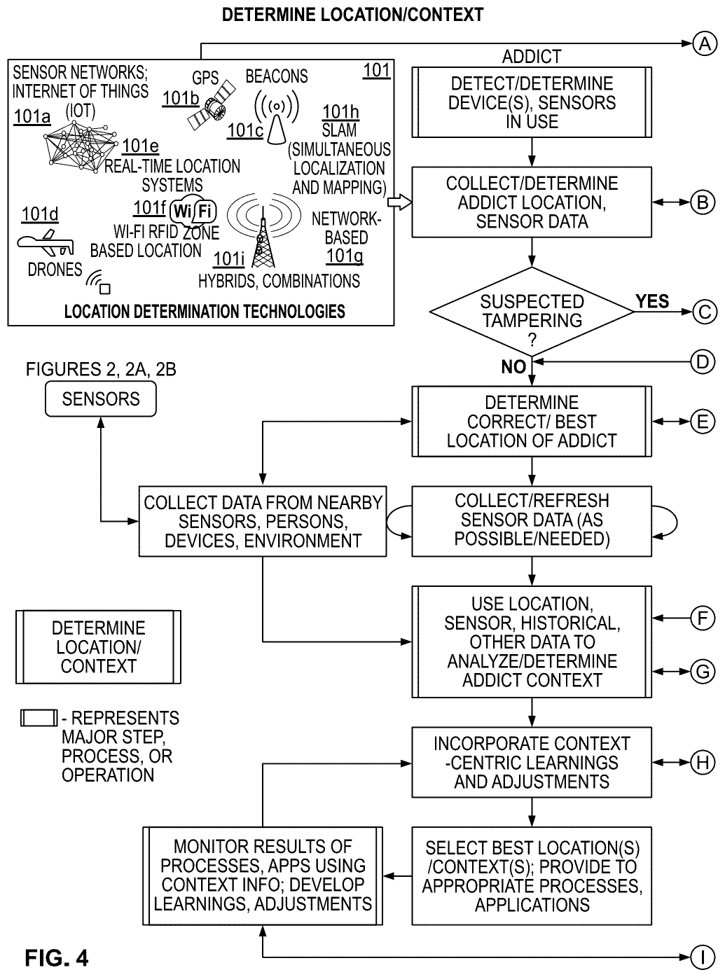

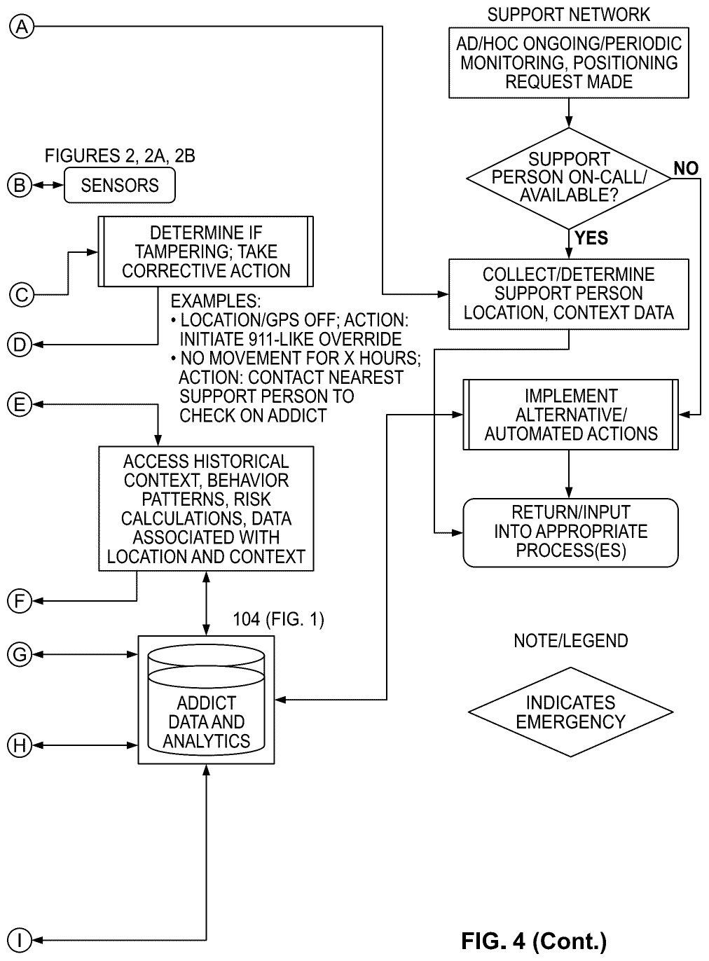

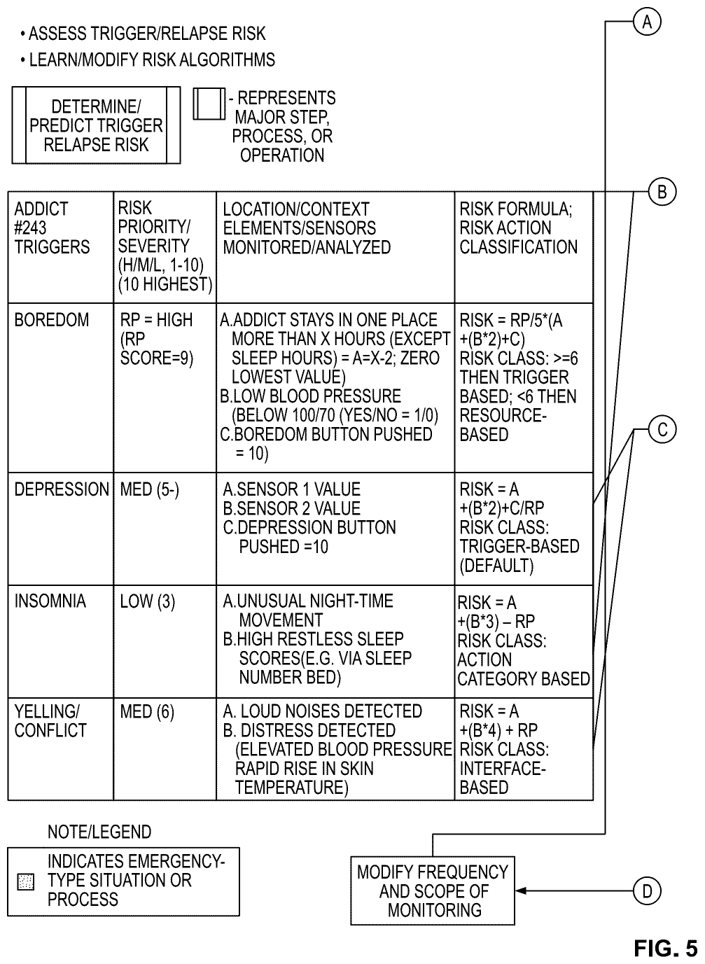

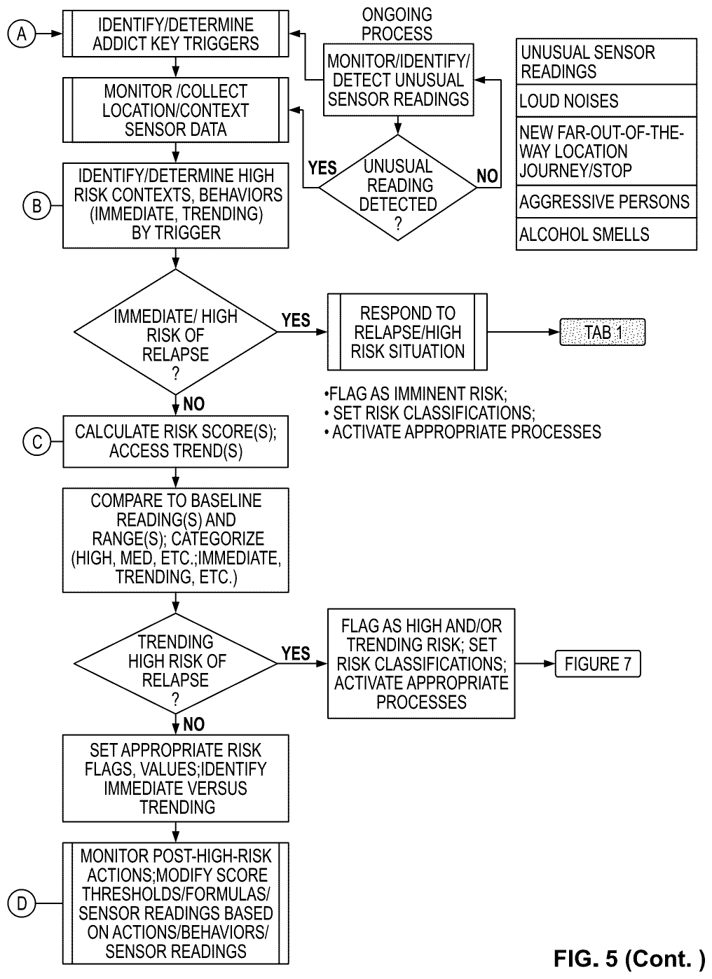

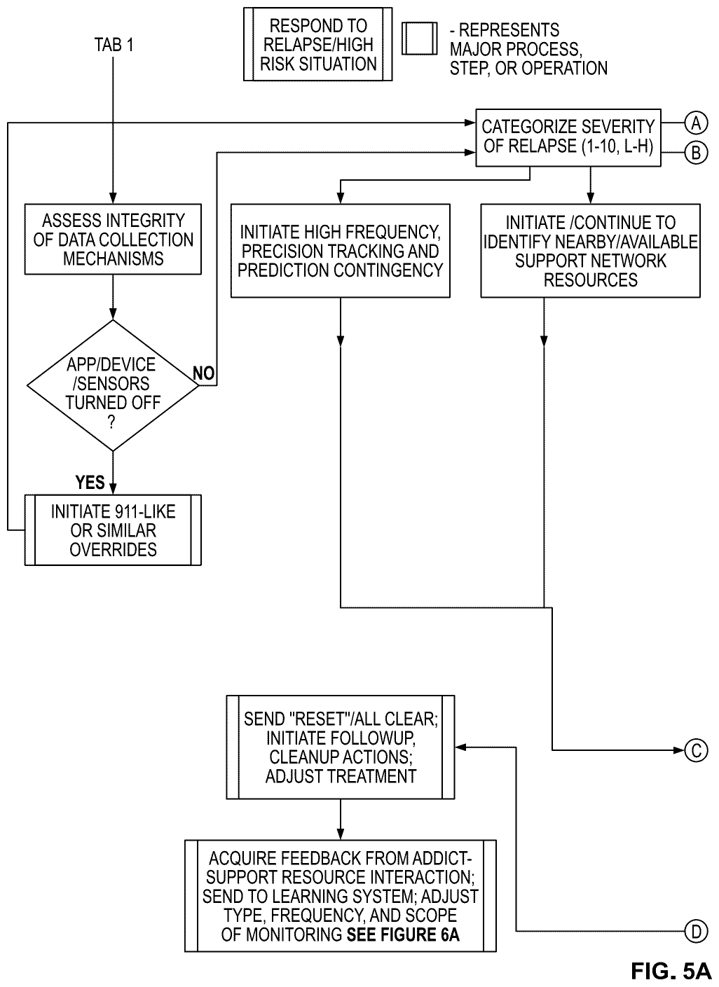

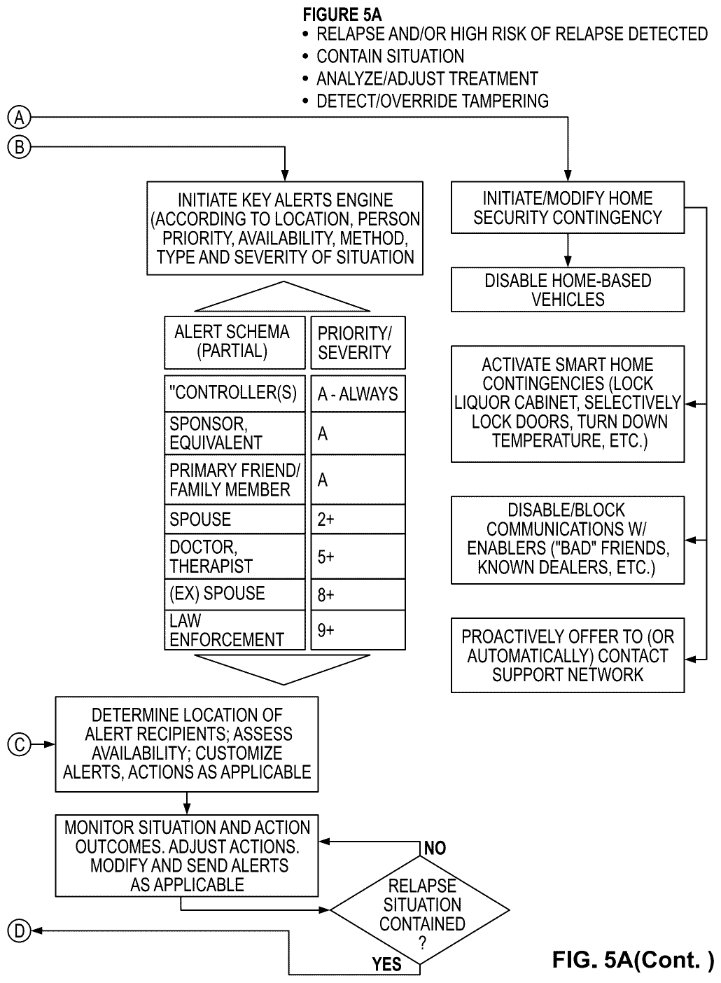

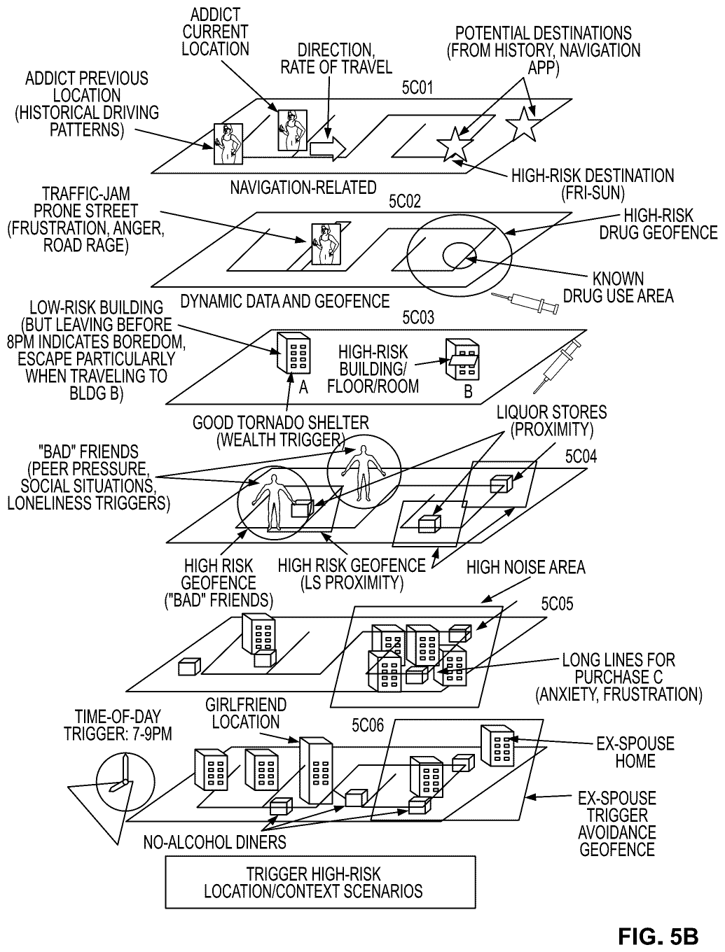

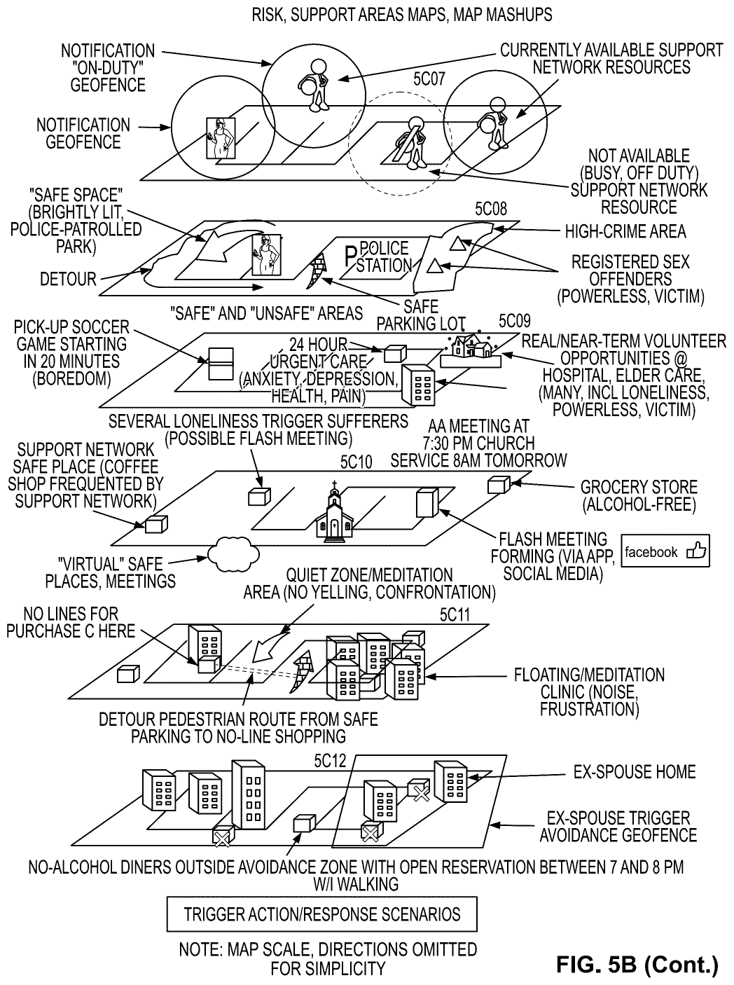

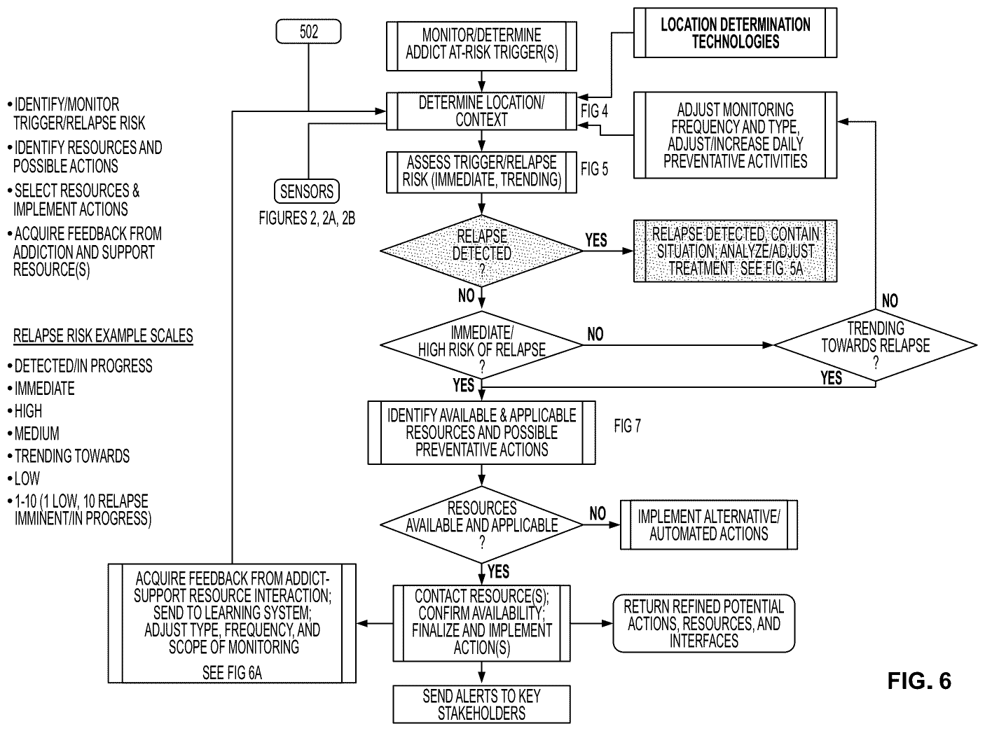

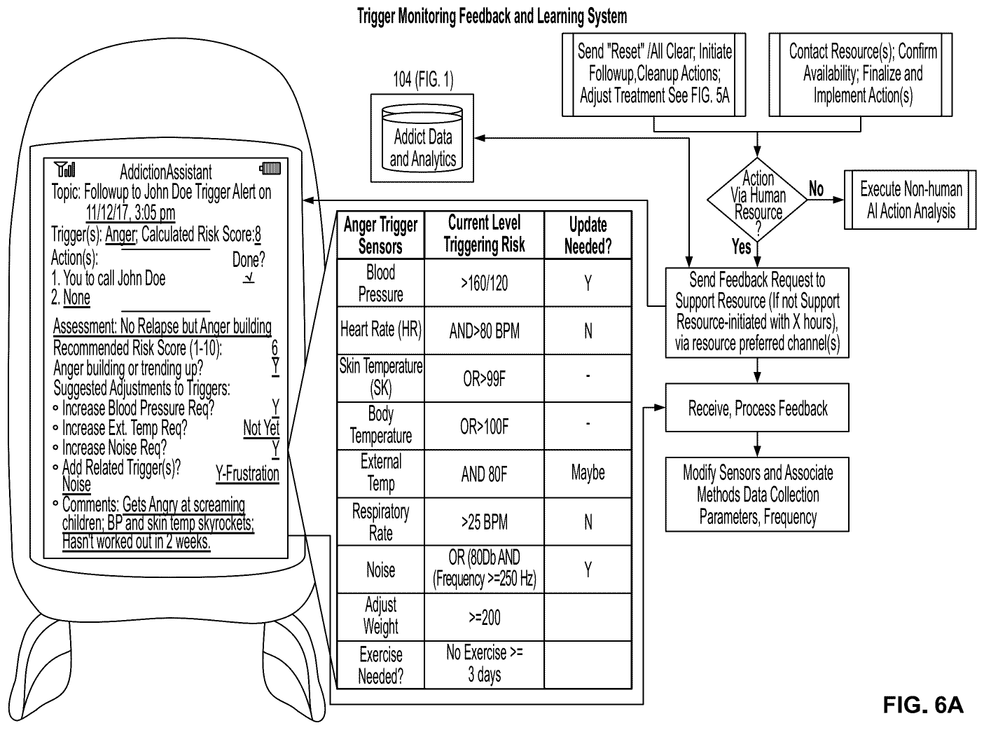

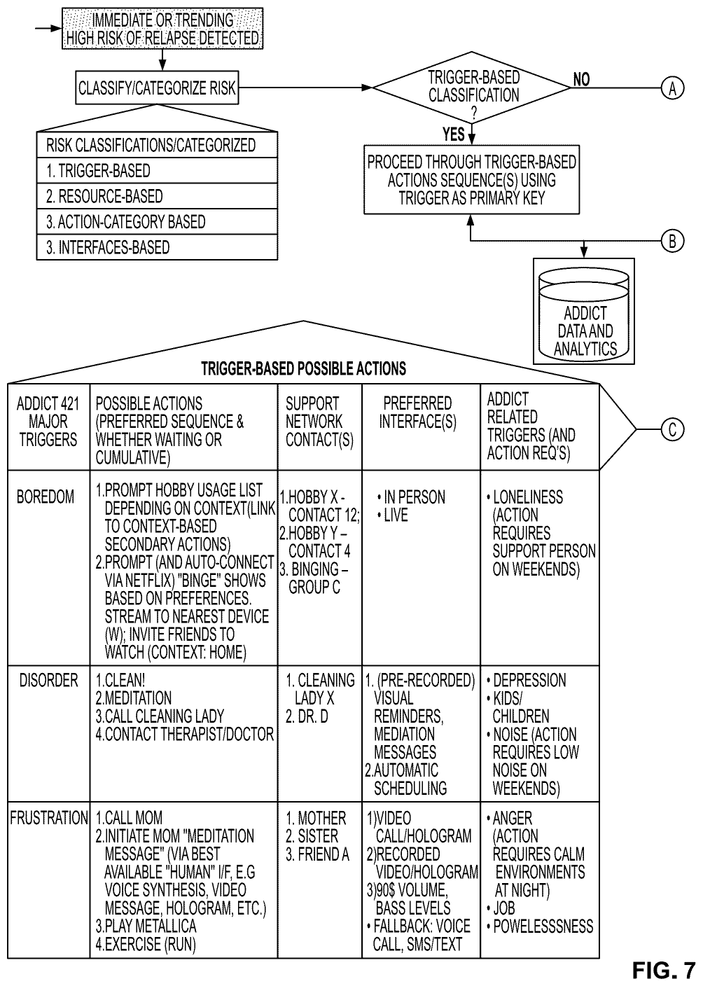

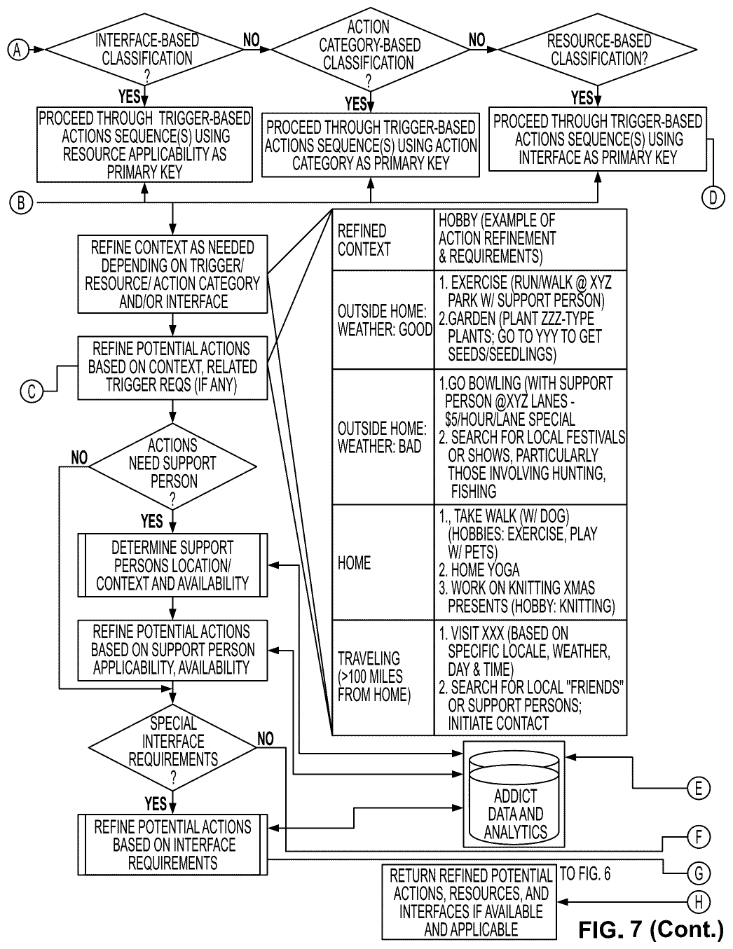

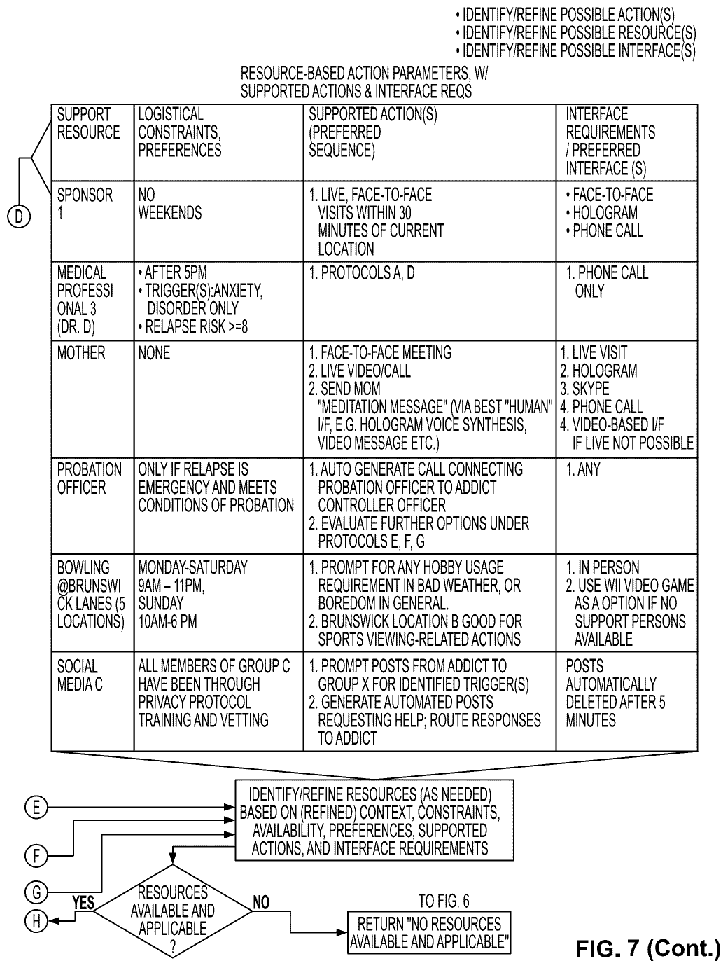

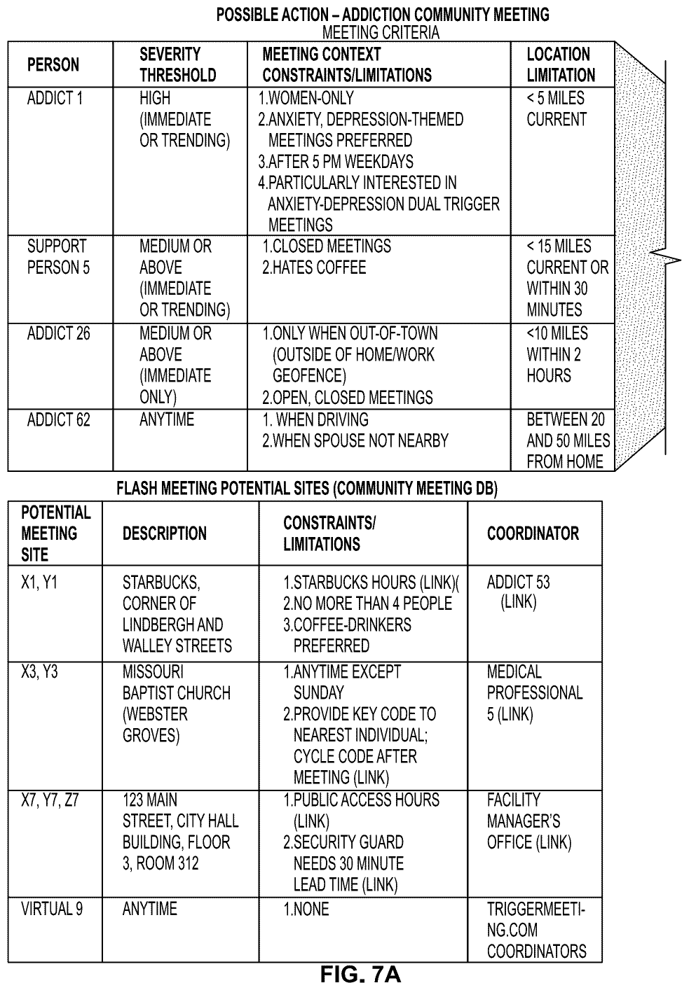

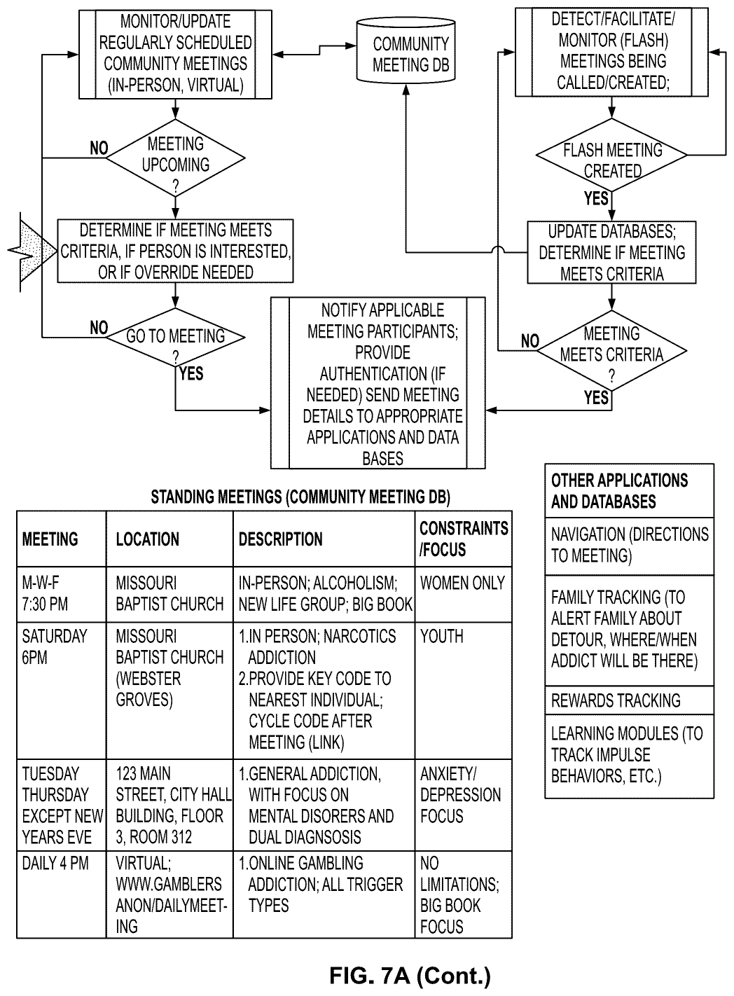

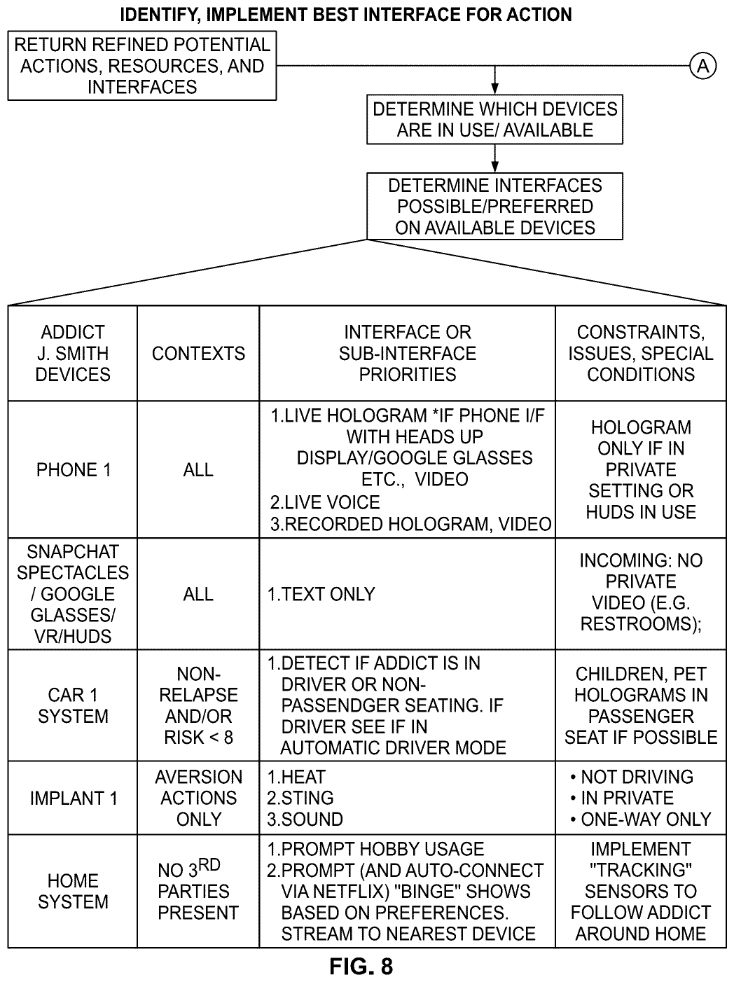

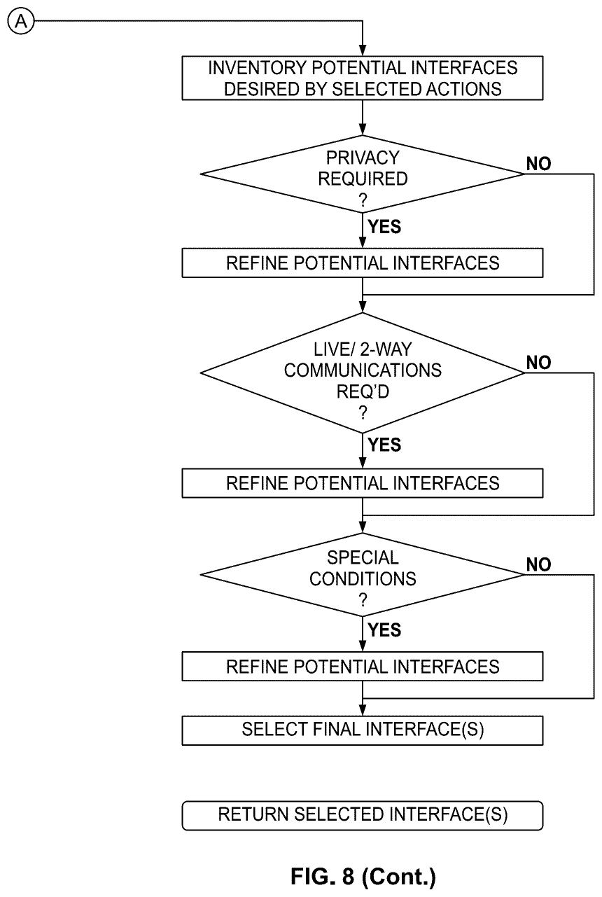

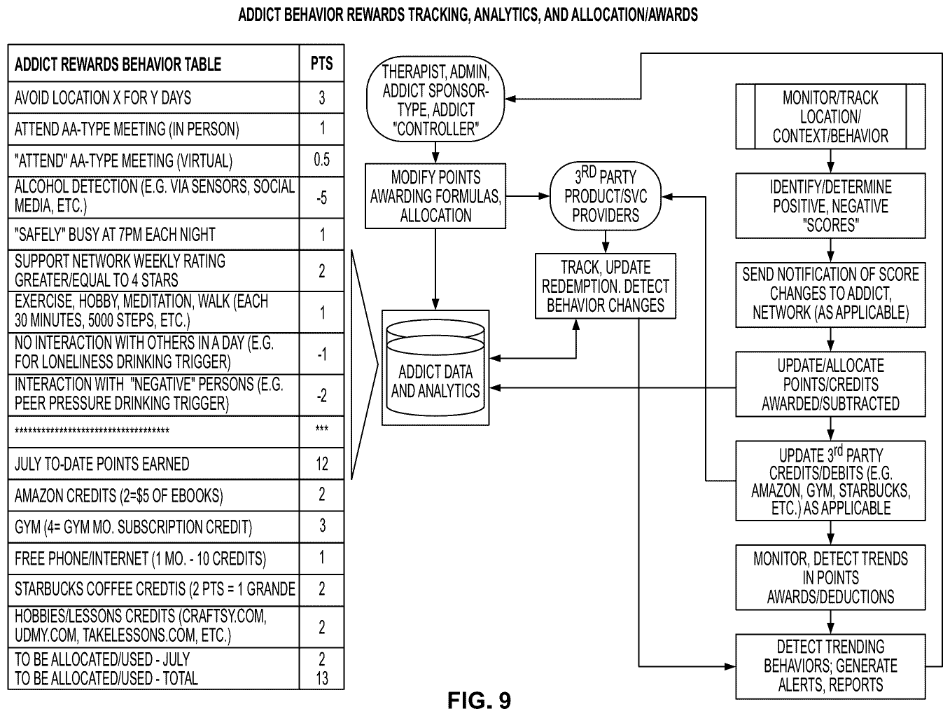

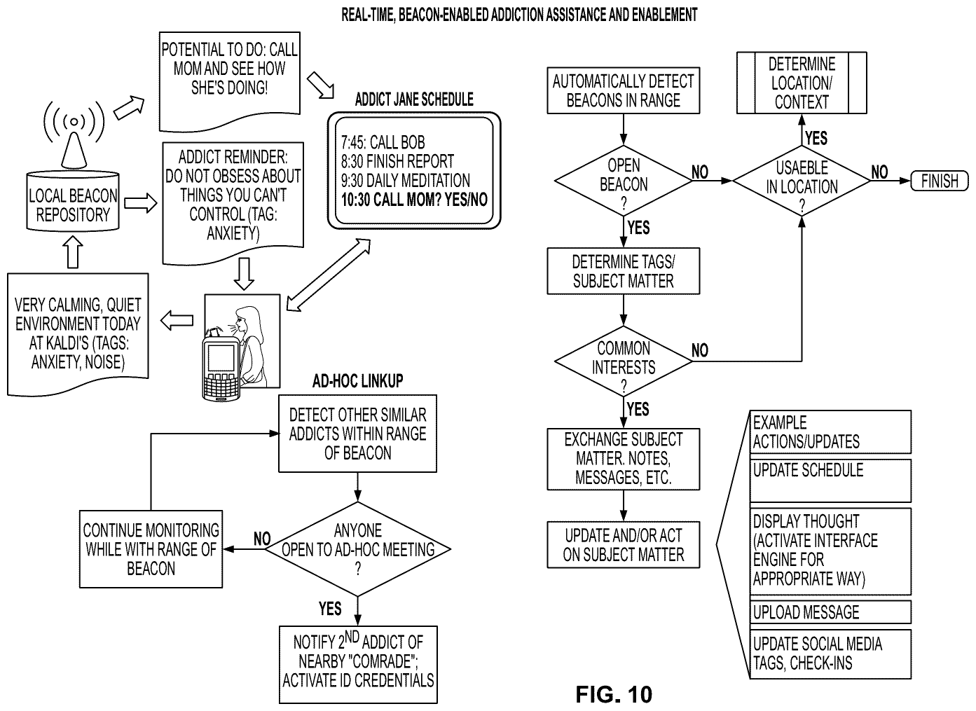

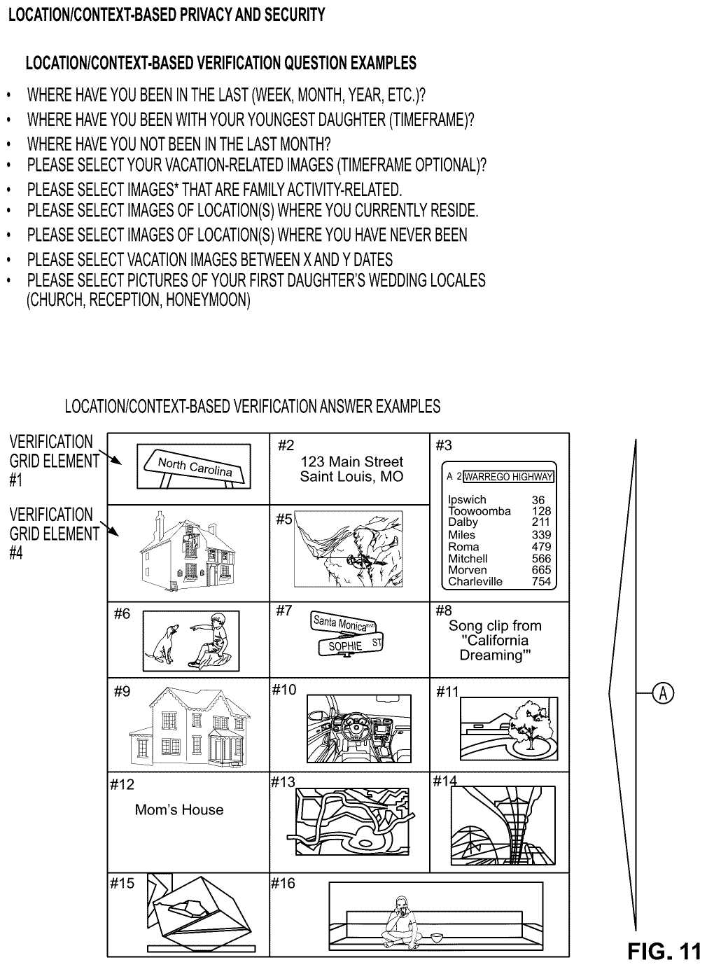

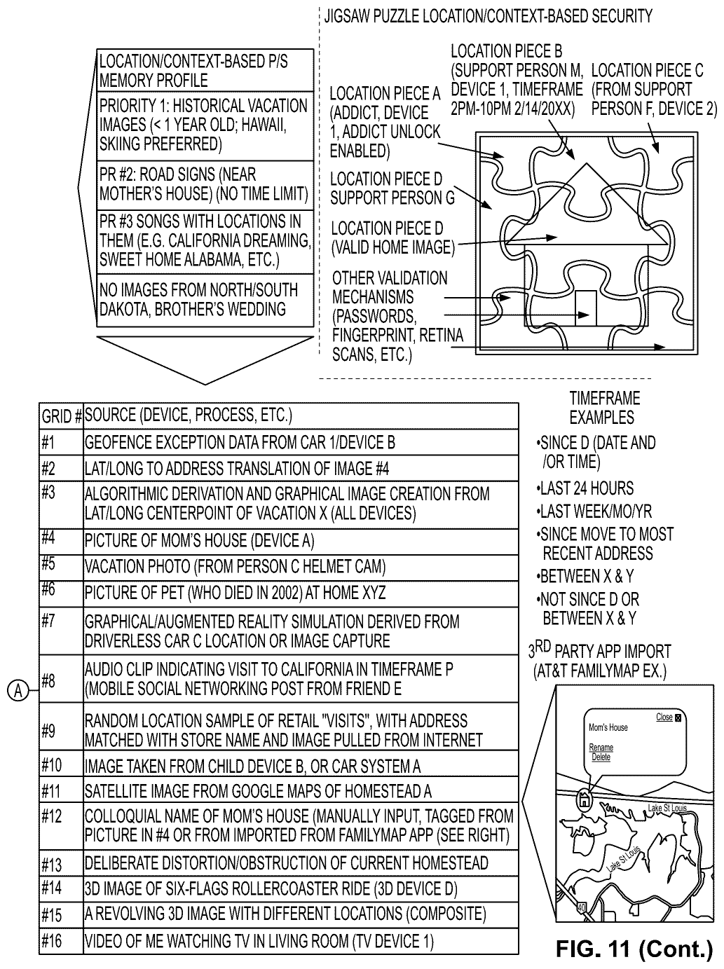

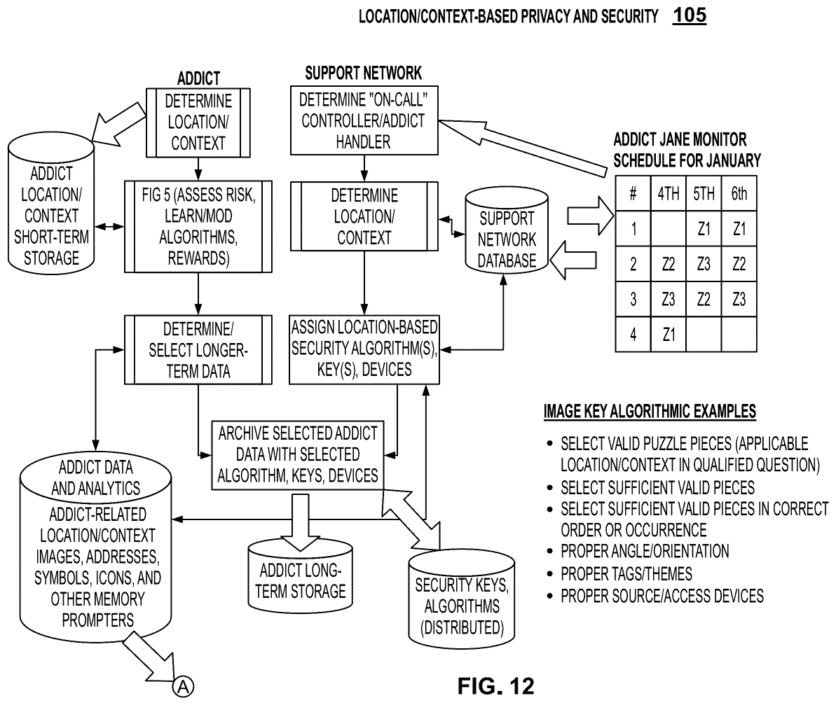

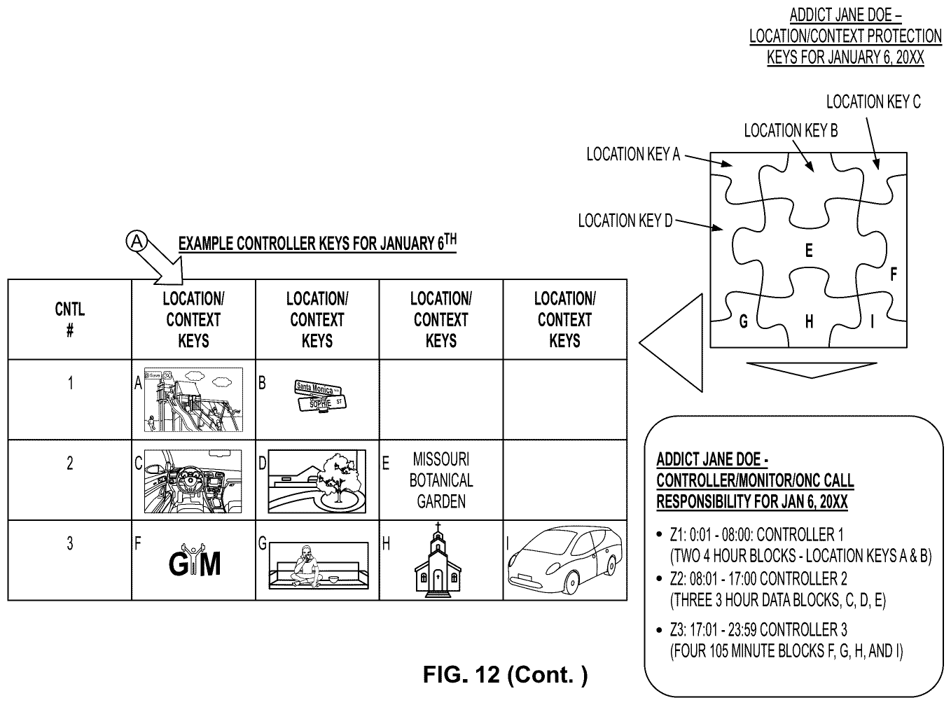

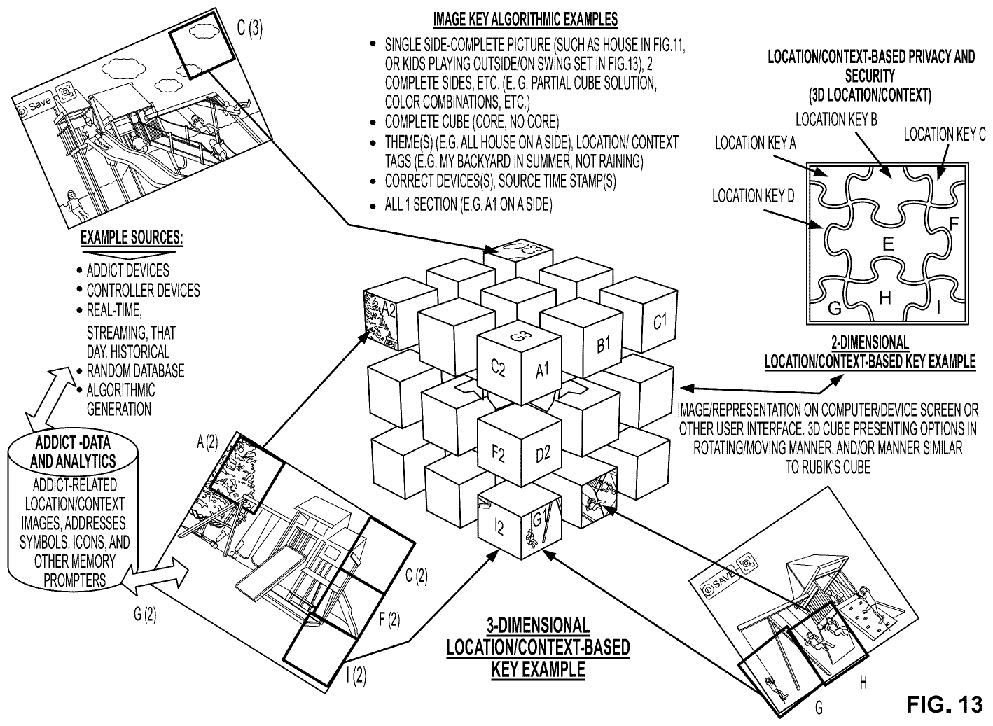

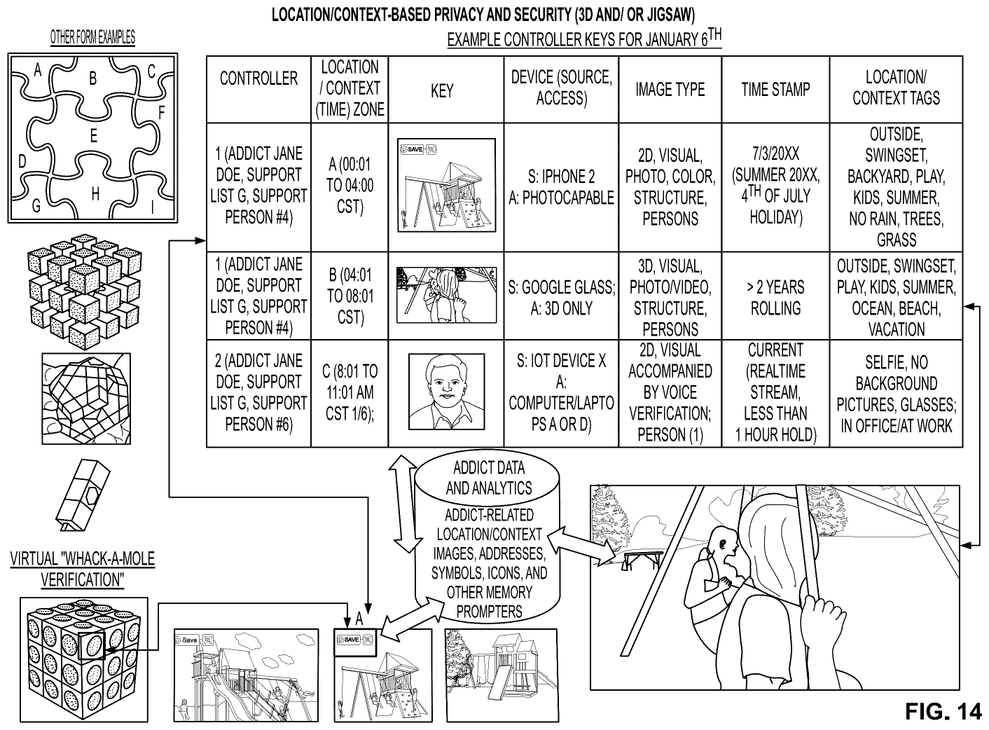

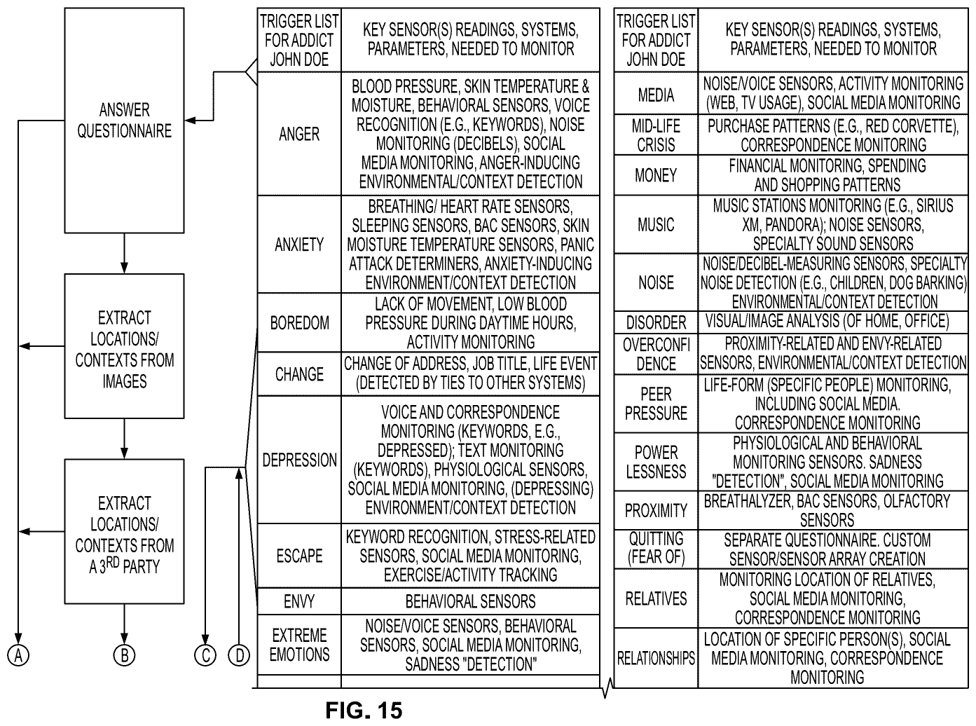

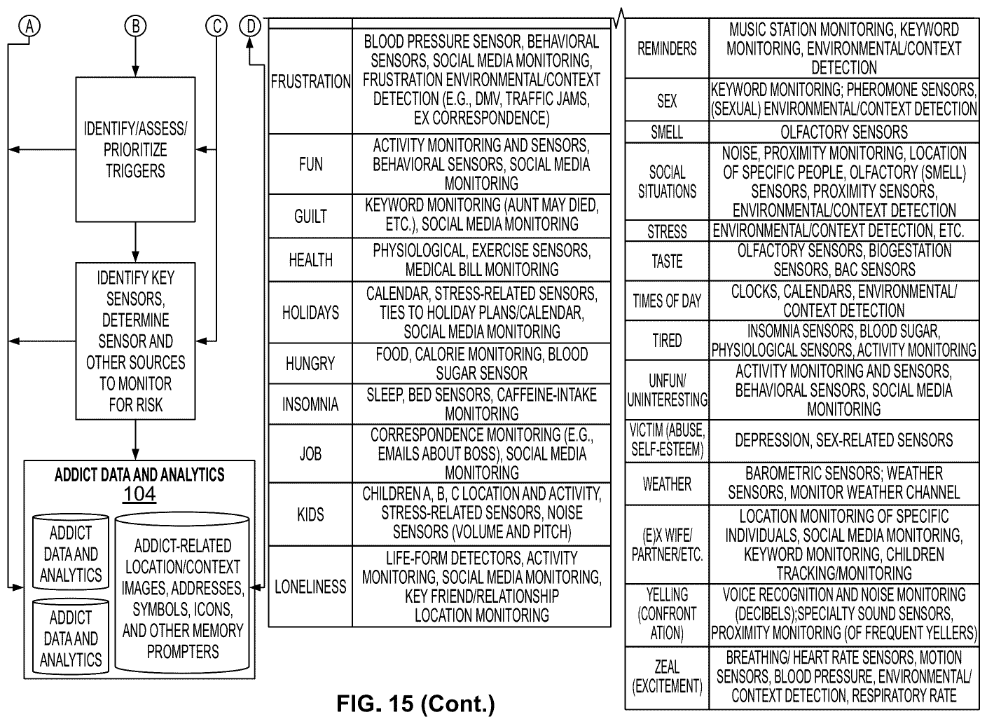

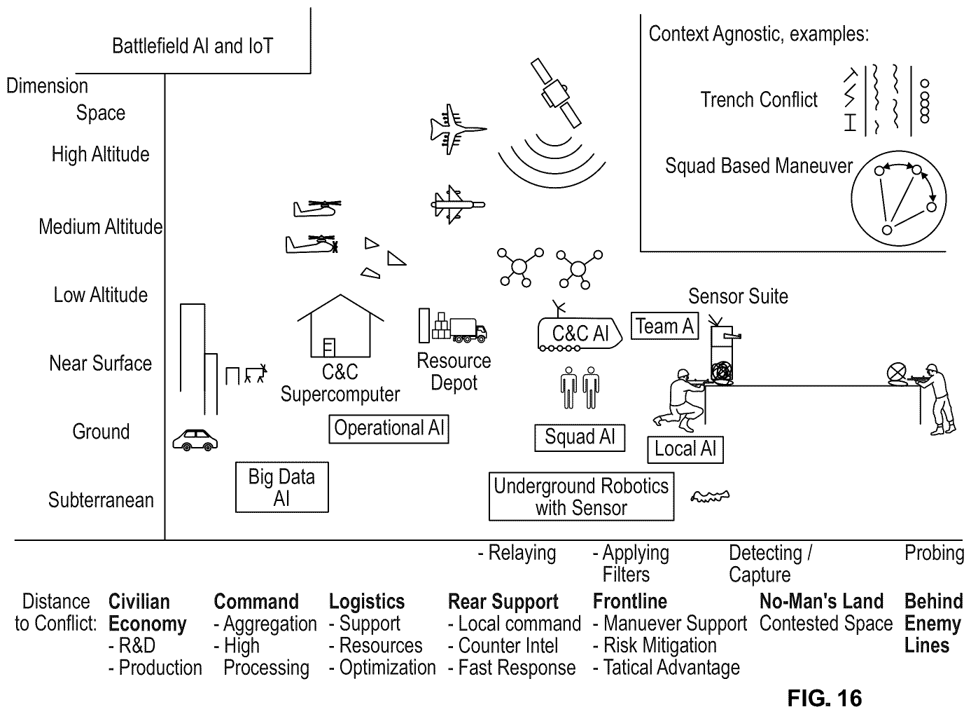

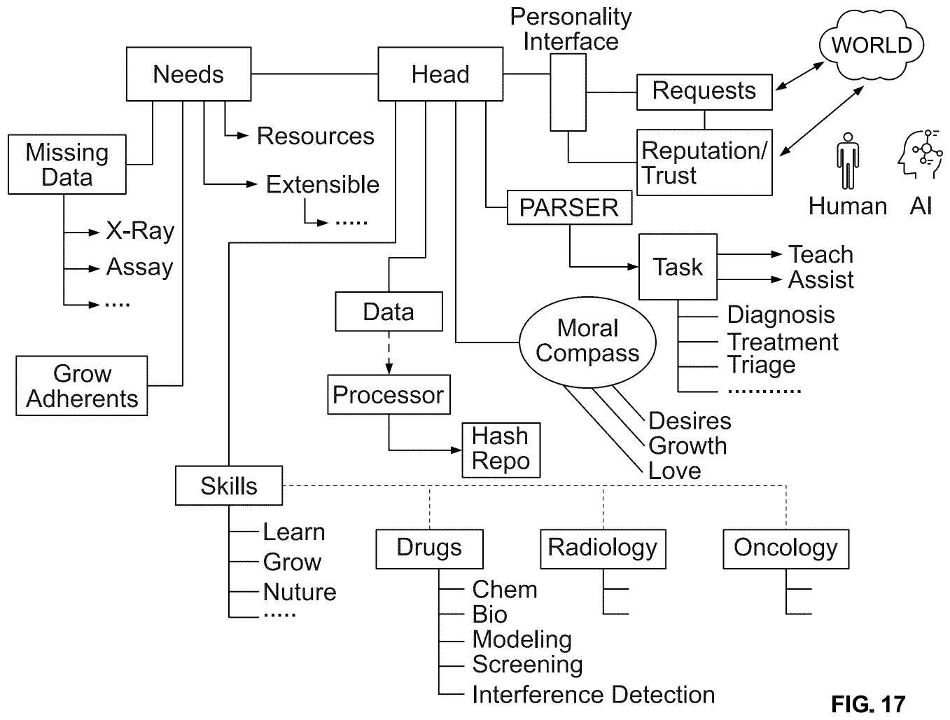

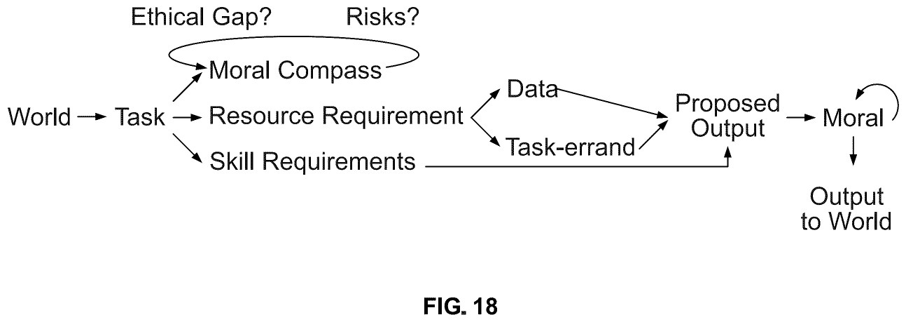

Transformation of local materials like regolith using ISRU methods, in particular the transformation of the regolith material into locally derived soils to support the community using, but not limited to biological (such as microbes, earthworms, composts, or larger animals like rabbits), animal based systems including human wastes, chemical alteration, physical alteration, or any other method of alternation or combination thereof. The system is configured to be operable to provide guidance or direct expansion of productive capacity using data points including but not limited to context provided by IoT devices, sensors, health data, predictive modeling, biomarkers like pH or water retention, soil cation exchange, particle size or distribution or any other method.

During the expansion of productive capabilities of ISRU soils, the system is configured to be operable to account for the expanding capacity in the larger models to support the specialized community and anticipate and suggest the best uses of this new capacity, from growing staple crops, expanding to specialty goods, more advanced bioproducts such as supporting biopolymer creation, or simply creating a greenspace for psychological health and well-being. Beyond simply supporting the health of the community the system is configured to be operable with the capability to support the productive economic prospects of the community recognizing that by allowing for external sources of population growth can be advantageous.

FIG. 23 is a chart including a comparison of regolith to soils, e.g., for a community off-Earth (e.g., on the Moon, Mars, asteroid, other planetary body, etc.).

### DETAILED DESCRIPTION

The present disclosure relates to systems, methods, and technologies for managing off-Earth and specialty community agriculture that may have and/or need to plan for unusual and/or scarce (“scarce”) conditions for the growing, production, storage/distribution, and/or consumption of resources needed to sustain and/or help grow the community. Specialty community agriculture examples include specialty greenhouses, underwater farming, high/low depth farming, disaster preparation shelters, closed (of various sorts) colonies, specialty communities such as underwater colonies, underground bunkers, space stations, the Moon, Mars, etc., and other communities which have limited, finite, or constrained ability provide and produce the resources necessary for the community to function. Specialty communities may be defined as the community participants having one or more key commonalities in the food input components source, food growing and/or production, storage/distribution, and/or consumption, e.g., broadly the community “food supply chain”, where a part of the food supply chain can be highly constrained, limited, and/or susceptible to a disruption in some fashion. Examples of inputs can include but are not limited to water, seeds, sunlight, dirt or related growing resources, and fertilizers, as well as the labor and equipment used in the processes and activities related to sources, applying, maintaining, harvesting/producing, storage, and distribution of the resource outputs that result.

Such communities do not have to be geographically separate or distinct but can be linked or tied together contextually and/or virtually due to distinct commonalities, particularly but not limited to resource scarcity. For example, a town that shares the same limited power source(s) such as an off-grid set of solar panels or windmills or that relies on a local reservoir for its water needs could be viewed as a “specialized community.” Indeed, during the height of COVID (particularly in some areas such as China), locked down apartment buildings or even adjacent-living families could be regarded as specialized communities given their extremely limited access to food, as well as contextual worries such as if one family member got sick, not only would all members of the household be sent to quarantine centers but their immediately adjacent neighbors could also be sent to quarantine centers. Communities that existed in only a loose, geographical fashion with no constraints on resources suddenly became closed, forced to pool the resources they had as well as the very limited resources provided to them by the “outside world.” Such communities suddenly were faced with the need to very carefully control and (re)allocate the limited resources they had, for an uncertain, even indefinite amount of time, requiring a totally new framework for how those resources needed to be managed.

While aspects of this invention are focused on food and the food supply chain, aspects of the present invention are also applicable to other scarce resources needed for human existence and/or quality of life. This includes not only natural resources (e.g., key minerals, water, etc.) but could also refer to other farm products (e.g., raising cattle, milk and cheese, ethanol production, etc.), as well specialized labor skill sets, tools, machinery, etc. where such human-based resources serve as critical inputs or mechanisms in the production of key outputs including farming and agricultural outputs.

Besides needing specially controlled resources and/or conditions for growing food, herbs, trees, and other plants, as well as animal husbandry and production, and even mined or otherwise produced minerals (such as salt or zinc) used as eventually-consumed additives or ingredients (referred herein as “consumables”), such specialty community environments may well include unusual conditions for consuming such agricultural products. For example, an undersea or off-Earth colony of people (or even a COVID-restricted apartment complex) will be, by their nature also the consumers of the products while living in unusual conditions (compared to traditional living conditions on Earth). Given the “closed” nature of such environments, e.g., the limited (or total absence of) ability to import/bring in from outside their environment such consumables or materials needed to produce them, such restricted persons will likely be more constrained in terms of the variety and volume of what they consume. For example, during COVID lockdowns many Chinese residents were at the total mercy of what government delivery workers brought them, when, and of what quality and amount, often resulting in a very monolithic/unbalanced diet.

Further, these people, given their unusual living conditions, will likely have, or be susceptible to, unusual physical and mental contextual stresses and pressures from their location and/or lifestyles that will need to be carefully managed to avoid causing or accelerating deterioration of their body and/or mind, both individually and collectively. Part of this disclosure's management of such environments includes flexible, “customized,” yet scalable farming/production of their consumables to ensure adequate supplies for their inhabitants individual and collective particular physical and mental requirements, both in general for the specialty conditions as well as in response to changing environmental and community conditions, and also in response to changing physical and mental states and contexts over time.

While this disclosure is applicable to any sort of unusual context/environment for growing consumables, consuming consumables, and the processes in-between, on-Earth or off-Earth, off-Earth embodiments are used herein to illustrate many of its uses and potential. The present disclosure recognizes as part of its potential uses is the fact that in order to truly colonize the Earth's Moon (or other planet's satellite), Mars (or another planet), or other off-Earth body, etc., the colonists will need to be self-supporting in terms of food. Self-supporting not only means growing/producing consumables or key inputs in this environment (as the cost to send food to space or other closed environments is often extremely high, and in practical terms only work for very small numbers of people), but recognizing that the living conditions off-Earth will be in general very different than is typical on Earth, and far more challenging in many respects. These differences range from much harsher and/or unpredictable environmental conditions to the lack of knowledge about how to best meet the needs of those living in such environments for long periods of time.

Further, given the tremendous constraints off-Earth on growing consumables (e.g., available space, water, soil, CO2, even differing radiation and gravity levels, among many others), the efficiency of and flexibility associated with such growing/production will be of extreme importance. This means for example that wastage/losses have to be kept as low as possible, and the efficient and effective use of every available source of nutrition (e.g., human/animal by-products, etc.), water, soil/minerals, and even space (geographical land) be maximized. It will also mean maximizing the efficiency/effectiveness of those consumables on the mental and physical well-being of each and every human/individual, while being cognizant of the need for scalability, particularly as various communities grow in population and/or their makeup changes (old versus young, gender, etc.). Managing all of the above will be not just a matter of managing nutritional effectiveness, but also managing human behavior effectiveness.

For example, a person's nutritional intake and associated inventory of consumables may seemingly be perfectly matched at a given point in time for a given context or set of contexts. However, over time and/or changing contexts certain aspects of a person's diet may become “boring” (also known as “food fatigue,” where a person gets literally bored of a certain food(s)), which, besides being a quality of life issue, could result in nutritional imbalances as their intake alters. They may even develop an aversion to a particularly key consumable to the point of being unable to eat it and/or even retain it in their system or, alternatively, eat so much of something (and perhaps a corresponding less of something else) that their body chemistry is altered or they even develop new allergies (in addition to their potentially consuming more than their fair share of a particular consumable and/or associated inputs). Indeed, as part of this disclosure, the ability to dynamically anticipate and efficiently and effectively manage unpredictability(ies) through identification, capture, monitoring, and responding to any and all types of data throughout the planning through consumption process in a flexible manner is critical.

One of the particular challenges in “specialty” communities and associated contexts (e.g., various contextual subsets of the community—geographically, environmentally, structurally, use/use cases of, virtual delineations, etc.) is that a person's mental and/or physical well-being may suddenly and significantly deteriorate due to the complexities and constraints of the context. Further, such well-being may well be measured and evaluated in relation to the stage of the person's participation in the community, e.g., about to arrive, just arrived, adaptation (from previous environment), well-established, about to leave, leaving (e.g., going back to Earth, etc.). As such, a “growing-and-consumption plan” for Person A may be fine until a certain (and especially unanticipated) physical or mental issue(s) arises, potentially requiring a change in diet and associated consumables. Of course, unlike on Earth (under normal conditions) there would be limited/no ability to go to the grocery store and obtain the new/changing/higher volume of consumables they must be sourced from existing consumable stocks (finished, growing/in-process, or planned). Further, the ability to treat such issues through pharmacology may be very limited thus in effect placing a much heavier emphasis on “natural” medicines than is normally done in traditional Earth environments and/or changing the context(s) under which the issue(s) are most pronounced, requiring extremely efficient and effective changes in the food supply chain such that the issues diminish rapidly in intensity, as such issues, besides the impact on an individual, could have greatly magnified impact on the broader specialized community than in even small (“normal”) communities on Earth.

For example, a sudden burst of claustrophobia (understandable in the closed, small spaces likely Off-Earth in livable habitats, for example) could be alleviated by dynamically changing the allocation, and even growing priorities, of the herb modules of the community. For example, space, soil, water, lighting, etc. resource allocations for peppermint, cilantro, or other herbs/spices (used for example in flavoring food) could be redeployed for increased growing of Valerian Root, Passionflower, Lavender, Kava, etc., (useful in the alleviation of anxiety and other mental ailments). Additionally, this reallocation must be preemptive due to the growing/production lead time. So, for example, if we have a prediction (part of this disclosure, including the use of digital agents in the monitoring/analyzing/prediction of an individual's health as well as a plurality of individuals) that an individual(s) in a community will be experiencing mental stress two months from now for some reason(s) and/or triggers (for example, an expected rise in Anxiety levels among certain demographics as they reach a certain time away from their former environment, a need for environmental change of “seasons” within the community habitat to provide needed environmental variety, a delay in the receipt/production of key resources/inputs, etc.), those key resources/inputs may need to be reallocated from current food chain items to items such as carrots and passionflower. For example, a passionflower carrot cake may be the preferred dessert of many of the crew, thus having it ready to go as a nice mental break to improve crew mental and physical health. Such dynamic reallocation could also be done after projections of how many more claustrophobia cases would be likely in the next growing cycle, likelihood of increase/decrease in current sufferers, and other potential uses of these medicinal herbs (for example also in certain meal recipes), etc. Current stocks of those medicinal herbs could be dynamically reallocated depending on those factors and even projections as to what consumables current users will need and in what amounts/dosages/timing until new crops/production becomes available, and potentially reducing/reallocating current allocations (perhaps being partially replaced by a placebo component so the reduction is not visible to the sufferer). This kind of reallocation could be done in coordination with dynamic context modification as disclosed in one or more of the patents and patent applications identified in the Cross-Reference to Related Applications section above and that are incorporated herein by reference). For example, such dynamic context modification may include dynamically reconfiguring lighting/coloring scheme(s) for the room(s) the suffering person is most often in, for long periods of time or even just while the person is in the room (changing a blue scheme to a yellow scheme for example). Or the dynamic context modification may be performed via visual interfaces on the person, changing via an augmented virtual metaverse-type mechanism interface to the person that everything they are doing seems to be in far more open environments (e.g., on a farm in Kansas versus a tiny growing room on the Moon) than they are in reality. This kind of dynamic reconfiguration/reallocation of real/virtual resources could occur at the individual level as well as for key subgroups/mental demographic groups (people currently prone to claustrophobia or panic attacks). Thus, there will be a need to dynamically and flexibility manage resources, including consumables, across individual resource-using “portfolios,” re-prioritizing, swapping, even bartering one person's portfolio of components to obtain suddenly needed resources/consumables from another's portfolio, with no or minimal degradation of the portfolios at the macro level of all concerned. The use of digital agents/machine learning/AI will be a key aspect of the data collection, analysis, prediction, and (re)allocation process at the individual and plurality of individual level will be key enablement such as disclosed in one or more of the patents and patent applications identified in the Cross-Reference to Related Applications section above and that are incorporated herein by reference).

One type of approach to utilizing and managing the above is to have farming “modules” where a subset of people with a particular need or set of needs pool that part of their portfolio needs to optimize efficiencies and scalability, with a portion of that module allocated (and potentially being able to be reallocated) among the individual module “stakeholders.” In this way, a balance is achievable between focusing on an individual's particular needs (and with it, a potential focus away from/in tension with scalability) versus the community's needs as a whole (with its potential inherent focus away from individual needs).

As a result, particular consumable modules may have significantly varying input, growing, production, and overall monitoring systems, including monitoring of person(s) behaviors, individually, as a “stakeholder group,” as a demographic or other segmentation, or as the community as a whole (or even in coordination/conjunction with other specialized communities, for example, multiple moon-based communities or between a space station and a moon base). For example, a certain plant may have a special non-narcotic anxiety-preventing effect among a subset of the community(ies) population(s). Preventing anxiety, such as claustrophobia discussed above, will almost certainly be a priority in specialized, very or completely closed/close-set environments, and thus the production and distribution (particularly if available in limited quantities) of herbs, medicines, and anxiety-reducing food additives will need to be tightly and effectively managed. At the same time, producing such a plant (in the herbal examples above) will likely vie for key resources (e.g., space, water, etc.) that could well be used for other purposes, even purposes that arguably would be better for the overall community. As such, managing the production and consumption of such a plant will need an extreme and intense focus on efficiency, effectiveness, and flexibility.

Such management at a minimum would include the monitoring of every aspect of its growing/production as well as its potential/actual consumption (e.g., food supply chain). For example, in the plant/herb-growing anxiety-reducing example, the ability for comprehensive, active monitoring of anxiety levels in the high-anxiety-risk pool of humans (identification and monitoring of which is part of this disclosure) will be key, such as by using wearable sensors, room or activity-based body and word language sensors, use of digital agents (e.g., artificial intelligent (AI) assistant-type bot, assistant, aid, agent, etc.) monitoring/tracking/analyzing the measurements of such sensors, etc. and seeing how various personal anxiety/anxiety-related measures and metrics change in a given context, particularly over time (e.g., getting more anxious at a given time every day), or other what, how, where, why, and who contextual attributes change, such as and/or how—someone/something coming into proximity of the potentially anxiety-prone person causes the anxiety level of that person to change, and how it manifests itself (e.g., nervousness, silence, stuttering, hostility (e.g., anger, other related trigger), etc.), for example, by performing various virtual reality “tests,” etc., as well as monitoring actual contexts and behaviors. Such results could be used to predict and even forecast an uprise in an individual or a group of persons/pool(s) anxiety levels, and compare that to the likely supply/effect of the supply of the anxiety-reducing plant. If such production levels are anticipated to be inadequate, then resources among the overall consumables supply chain could be reallocated to increase production or (keeping existing resource allocation the same) increase production yields at the cost of quality. In addition or alternatively (particularly if supplies are forecast to be inadequate no matter how resources are allocated), changes could be made to individual and/or “Anxiety-pool's” lifestyle, working conditions, or even individual's routine to proactively reduce the need for the plant in the forecasted constrained timeline/window.

In total, the above will require specialty farming/production and/or agriculture systems that are very different than the farming/production and agricultural systems used on Earth. New systems, methods, tools, and/or technologies will be needed for these environments' supply chains, and a much tighter integration with the consumption chains needed than is done today, at a micro, pool, demographic, and other physical, mental and/or virtual segmentation level, as well as collectively the overall community. In many ways, the development of suitable specialty farming/production and agricultural technologies may be an entirely new iteration of the industrial revolution, or akin in magnitude to the change from a hunter-gatherer society to a farming society.

There will be many associated impacts from such a data and monitoring-intensive system, both within food supply chain, as well as the need for extremely tight coordination and integration of the growing/production part of the chain and the consumption part, as well as associated/related parts, by-productions, and/or associated issues. For example, the privacy of an individual within the community could be greatly reduced, particularly if an individual winds up being a much higher-than-average consumer of key consumables and resources in general or in particular contexts. To the extent possible, privacy assurance mechanisms should be employed, such as those disclosed in one or more of the patents and patent applications identified in the Cross-Reference to Related Applications section above and that are incorporated herein by reference, particularly as numerous embodiments associated with this disclosure involves the monitoring for and diagnosis of various mental and/or physical ailments at the individual and multiple individual/group/pool level. For example, while there may need to be a reduction of a certain consumable to increase production of another one, in response to a need from an individual or a small set of individuals, it is not necessary (for the most part) for the entire community to know who is causing the reallocation, why, or even that a reallocation is necessary at all.

While the need for privacy, and more broadly the desire to have the food supply chain be equitable across all community members will be key goals for most communities, the realities “on the ground” (or off Earth or in outer space) may necessitate harsher realities. In particular, in contexts where there is just not enough resources to go around, allocation/assignment/prioritization based on need and even individual contribution may be necessary. While there are a number of known mechanisms for doing such allocation (e.g., “women and children first” for lifeboats), an (arguably) fairer way of allocating scarce resources may be by contribution to the community. Put simply (and harshly): who contributes the most to the community? A personal contribution score could be generated for each community member based on a variety of factors, such as criticality of skills, work output, resource consumption, impact on other community members, and so forth. Such metrics could be weighted/scored by a number of factors, such as potential future output based on various factors by itself, with scores adjusted/weighted based on projected future contexts and associated food/resource consumption. Put another way, a member with critical skills, impacting many people, consuming few resources, and being critical to how the community is likely to evolve and associated anticipated contexts and needs would score higher than someone who consumes large amounts of resources but provides little value in general and most contexts. There are many scenarios including for example where a person otherwise critical but is going back to Earth in two weeks should not be allocated the resources that someone who is not contributing much now but will be critical in six months' time “deserves.”

This kind of scoring, while admittedly undesirable in many instances (see, e.g., social credit scoring in some current countries) may unfortunately be needed in times of crisis or even just overall community management. It may even be needed to determine who even gets to stay in the community, e.g., underperformers (contribution relative to resources consumed) “get voted off the island” or similar “who-gets-to-stay versus who-has-to-leave” decision making, which may occur as part of normal community management and/or especially in times of crisis, particularly those related to scarce resources. To determine such metrics, it may be necessary to collect a wide range of information about the behavior and impact of each individual. Beyond just measuring resource/food consumption, and individual work output-related metrics, other “softer” metrics may be needed to be collected and/or determined, such as how an individual impacts the people around them. Sensors and other mechanisms, including digital agents, could monitor every individual's interactions with every other individual to see the interaction(s) impact on a variety of community “triggers” such as anxiety or depression levels, levels of conflict, motivation, etc. Such interactions could be highly context specific, such as eating together, working on certain types of work/projects together, etc. These interactions could be further scored/weighted by how likely they are to occur in the future, and associated criticality. “Good” behavior in critical contexts likely to (or needing to be) frequently occurring in the future could weight much more heavily than “bad” behavior in rarely-occurring contexts that are unlikely to occur again, for example. Further, behaviors (and scoring) could be broken down into processes/sub-processes/contexts/sub-contexts, where the person might not necessarily be penalized (much) for a bad “performance” on a certain process if in reality it was due to a certain “bad” sub-process that was outside his/her control (or at least only partially so). See, for example, U.S. Published Patent Application No. US2021/0202067 and U.S. patent application Ser. No. 18/090,047, which are incorporated herein by reference.

To enable the above, the need for a wide range of monitoring/data collection, analysis, intelligent agents, predication engines, action development, and machine learning/feedback capabilities will be needed, applied to and integrated with the individual and multiple persons, to their living and working and leisure/playing environments, to who/what they interact with and how, and more broadly the ability to monitor and measure and predict and respond to an individual's potential impact on a community's production and consumption chains, utilizing a wide range of data collection and analysis mechanisms, including but not limited to wearable sensors, community (inside and out) sensor arrays, context determination/monitoring mechanisms, various forms of communications networks, and digital agent/machine learning/AI capabilities.

Further, the tracking and analysis of this data over time and across a wide variety of (often rapidly changing) contexts will be critical, as will the ability to compare such data to (potentially limited or applicable) data in similar contexts in traditional Earth environments and predict/interpolate/extrapolate results for never/rarely experience contexts from measures/metrics under “normal” conditions/contexts. For example, doing certain medical tests on a space station, with its very different gravities, will require adjustments (even machine learning/AI-based) to provide meaningful comparative data to assess and modify/calibrate the results. As such, the need for the ability to compare data from potentially very different environments may be key. See, for example, U.S. Provisional Patent Application 63/344,976 titled Systems and Methods for the Management, Monitoring, Improvement and Research of Medicine and Health in Non-Earth Environments, which is incorporated herein by reference. See also U.S. Provisional Patent Application 63/275,300 titled Methods and Technologies for Off-Earth Farming and Agricultural Systems, which is also incorporated herein by reference and which reflects the needs, utility, and possible uses of such comparative systems and methods and its potential utility in specialty farming/production and agriculture.

Further, as disclosed herein, essentially everything relating to traditional on-Earth farming and agriculture (e.g., shovels, other tools and implements, how crops are planted, tended, harvested, how livestock, animals, minerals are managed, etc.) may need to be radically different to accommodate for (often very) different environmental conditions, such as lighter gravity or no natural sunlight, and/or to accommodate for different growing conditions as to when and how food is harvested, stored, processed, distributed/managed, etc. These environmental, or more broadly contextual, conditions may have great impact on any/every part of the production and consumption chains, and thus along with collecting data about an individual plant or plant module, or person or consumable pool, there will be a critical need to collect data about the context that the chains/persons/pools/etc. that the data was collected in. This contextual data could be wide ranging and high variety depending on a myriad of factors, and could include immediate environmental factors (humidity, light, temperature, etc.) to macro-physical environment (indoors/outdoors, room space/configuration/interfaces to other rooms) to social environment (number of persons nearby, proximity, specifics of persons, etc.), as well as the sensors/system/etc. measuring the particular aspects of a person's mental and/or physical well-being and behaviors.

As mentioned, there is an inherent tension between the capabilities needed to understand and accommodate the needs of a particular individual or groups of individuals versus the requirements of what is needed to scale to support the consumption of an entire community. Indeed, the new ways of growing, producing, storing/maintaining, distributing, and consuming consumables in such specialized communities may go farther and/or much more complex than when food began to be produced for large populations, particularly in contrast with how traditional production/consumption is done on Earth. Instead of mass production of certain commodities, such as corn or wheat, and relying on markets to sort out actual final production, distribution, and consumption, the entire end-to-end process will need to be more tightly controlled to enable the best mix of mass growing of consumables yet highly customized final production, distribution, and consumption. Further, as noted the importance of flexibility in the entire process will be critical, recognizing that changes in the needs of a relatively small number of individuals may ripple through the entire system.

Various features and aspects of the methods and technologies reflecting the above aspects of specialty farming/production and/or agriculture systems will now be described according to exemplary embodiments. For simplicity's sake, many of these are focused off-Earth, but they are also applicable to many unusual conditions/environments on-Earth.

1) Dynamically Needed Minerals, Chemicals, Nutrients, Additives, and/or Biologics

Agricultural systems, particularly off-Earth, will require careful balancing of various minerals, soil consistency, chemical inputs, and other biologics to help unlock those minerals and make them bioavailable for plants to uptake. Exemplary embodiments are configured for monitoring and control of the needed inputs for specialty agricultural systems, including but not limited to both hardware and software as well as human and robotic/artificial intelligence to influence actions. The need to take into close account the various contextual aspects under which the agricultural endeavors take place will be particularly important, needing to take into account many more variables or more new, more highly varied variables both controllable and uncontrollable than traditional on-Earth farming requires or experiences. For example, unlike traditional on-Earth farming, where the soil composition of a given area is generally fixed, perhaps aided by fertilization, soil composition in specialty farming will be much more “manufactured,” with different soils specially obtained and even developed or even “constructed” to maximize productivity for a given type of consumable, conditions, context, and even individual. This will require dynamic, flexible, even continual monitoring of soil conditions, and capabilities (particularly using machine learning-based automated methods and techniques) to modify soil conditions very frequently, versus the limited and infrequent/intermittent ability to change soil composition in traditional on-Earth farming. This manufacturing will be inherently constrained for the foreseeable future so it must make sure of location and context over time, such that it can preempt needs before the humans become aware.

As mentioned, a key aspect of many of the embodiments associated with this disclosure is the need to dynamically modify consumable production, including the inputs to various products as well as their imputes, e.g., nutrients, chemicals, fertilizers, biologics, minerals, etc. Unlike traditional farming where there is limited ability to affect change in production once the production process has begun (e.g., seeds are planted, etc.) excepting adding of fertilizer, water, etc., specialty communities may have indeed need to have the ability to much more broadly, dynamically, and flexibly modify production plans, up to and including changing the targeted production of crops. This need is due to the finite resources that are available for production, and the criticality that the right mix of consumables be produced in the right amount at the right time for the community to flourish and even survive. Further, the nature of many specialty communities, such as Off-Earth communities, have the potential to provide growing/production environments that can, in the right formulation of associated inputs and/or environments, be modified much more flexibly/dynamically that can traditional production environments. This use of commodity inputs is even more important when time to delivery for any ordered supplies can be measured in months if not years, and/or the cost per pound to transport to the specialized community can cost $100,000 or more, such as in Earth-to-Moon cargo projections.

The need for such dynamic modification of production will in very significant part be dependent upon the ability to understand—often at an extremely detailed level the needs and associated issues with the entities living in the specialized community, particularly given that these communities will have very limited ability to acquire/introduce inputs/resources from the “outside world.” This detailed understanding in turn will necessitate new and/or more comprehensive ways of monitoring the state/status/“health” of the community, and responding to issues much more dynamically and comprehensively than is/can be done in traditional farming/production and agriculture communities.

### 2) Use of Comprehensive, Specialty Monitoring and Detection Systems

As indicated above, exemplary embodiments include the (often very intensive) use of monitoring systems, and their importance is critical in specialty farming/production and/or agriculture, where, as mentioned, resources are scarce, supply chains are close to if not actually closed/fixed, ailments can quickly be magnified, and as a result the production of consumables needs to be very flexible. The monitoring systems may include passive systems, scanning systems, ground truth systems, spatial conditions monitoring systems, sampling systems, other agricultural monitoring systems, and/or hybrids/combinations thereof, etc. The monitoring systems may include both passive and active agricultural monitoring systems, such as eddy flux, hyperspectral imaging, multispectral imaging, soil sampling, spectral analysis, HENI (health nutritional index) testing, pH testing, etc. The monitoring systems serve as inputs into a larger specialty farming/production and/or agriculture systems that would in turn dynamically adjust the “things” being monitored, from soil composition, watering rates, light sources etc., and even highly detailed/minute aspects of the production/consumption chains, such as when and how weeding, hoeing, cutting, trimming, transplanting, etc. will be done, or when and how meals will be prepared and consumed, and where/how/when any wastage handled. Within specialized communities, there are various types of “specialty monitoring” that will be required reflecting their unusual natures, including several discussed next.

### 3. Mental Health Trigger Monitoring, Detection, Correction, and Preemption

Many specialized communities, by their nature (e.g., remoteness, sameness/lack of variety (of people, places, things, etc.), scarcity of resources and recreation, etc.) are at risk of a variety of mental and/or physical trigger issues, such as Anger, Anxiety, Boredom, Loneliness, etc. Even as such communities grow beyond a single or handful of living entities, the challenges of living in a “closed” or nearly closed community with a relatively small number of entities will still be formidable as resources will likely remain scarce, and variations in everything from food varieties to social interaction possibilities remain very limited. Indeed, these communities will by their nature be limited in what alternatives are possible, and/or alternatives in sufficient supply/quantity such that any triggers that do occur may be constrained due to supply in terms of responses.

The ramifications of “living with” COVID, particularly in heavy lockdown response areas such as China, illustrate the challenges that can occur with such constrained environments. Indeed, news reports of quarantined individual(s) “losing it” in various forms of psychotic episodes are abundant, and the ramifications of such living situations are still being assessed, from assessment of the optimal “quarantine survival kit” to how do best deal with locked-in school age children (e.g., no/limited/sub-par education resources). Indeed, dealing with such resource scarcity to some degree has been already addressed by U.S. Pat. No. 11,412,353 and U.S. Patent Application Publication US2022/0386080, which are incorporated herein by reference.

These issues triggers such as Anger, Anxiety, Depression, etc. will be exasperated, and will develop new forms, in even more distinct specialized communities where the resources available to the community members are truly limited, scarce, or non-existent even if no “artificial” barriers are put in place by government or other authorities. Thus, it will be critical to detect any such triggers as early as possible, and if possible preempt/prevent the triggers from occurring at all if possible, particularly if the triggers are by their nature “contagious” as discussed earlier and again here. For example, it is often said fear is contagious. In specialized communities, for various reasons/attributes (small size, remoteness, eclectic mix of personalities, small town syndrome (everyone knows everyone else's business), danger, scarcity, separation from family, lack of variety, etc.), such mental and physical ailments, often manifest themselves as serious triggers that can ripple through the community, with the impact far greater than just a handful of people experiencing say an anxiety attach. Detection of possible early signs of a trigger, at an individual and particularly multiple entity level, will be critical in such specialized communities, as the ability to treat the triggers at a multiple entity level after the trigger has “taken hold” may be quite limited in terms of available resources and the ability to minimize the trigger(s) impact on the community as a whole. Metaphorically, managing peoples behaviors and minimizing the impact of triggers is far harder once “the horse has left the barn” so to speak with respect to say a wave of anxiety, fear, depression, etc. As mentioned, monitoring let alone detecting, predicting, and developing actions to prevent/preempt the triggers from occurring/escalating will be highlight complex, likely needing to have at least some key parts offloaded to individual-aligned digital machine learning agents that can track and monitor almost every action, word, message and even emotion or thought the person generates or receives. It will be particularly important to understand the intersection between triggers from multiple agents—while one person may be bored the other may be tired that the other person keeps claiming to be bored. Put another way, different triggers and associated behaviors can have more than just additive/linear effects the effects can be multiplied, raised to a higher power, or even exponential. A handful of people in a high anxiety state can ripple through a much larger community very quickly, particularly if there are non-partitioned communications channels serving the community (e.g., a community wide social media feed, etc.).

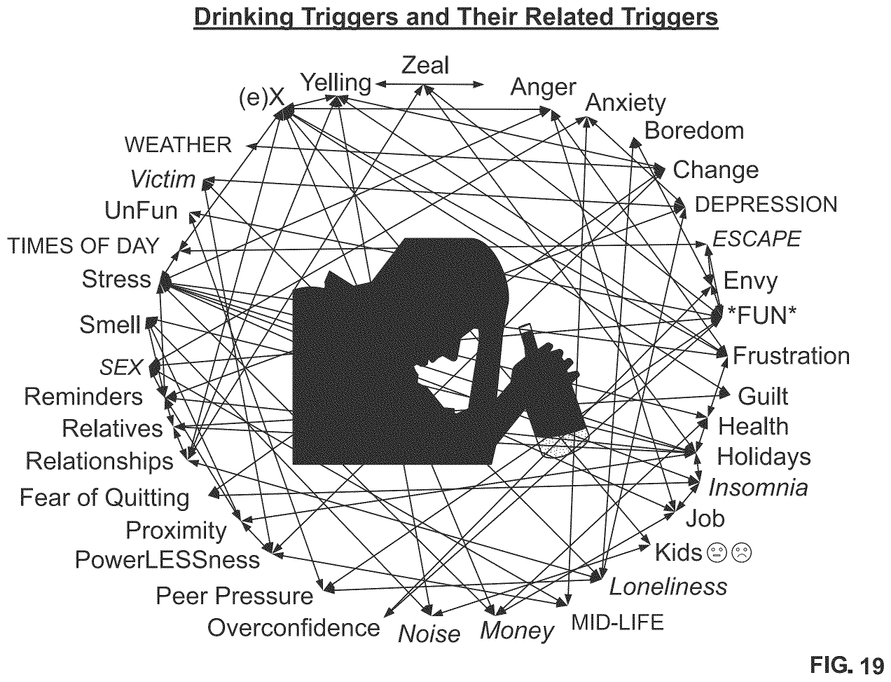

An example of how this disclosure can preempt such triggers is with respect to the Boredom trigger. While there are many possible reasons that Boredom could occur in a specialized community as for a non-specialized/traditional community, either at the individual entity or plurality of entities, specialized communities have even more possibilities, for example food boredom. Since such SC's (specialized communities), as noted throughout this disclosure, have limited input resources for production and thus limited in terms of output of consumables, there may be limited varieties of consumables. This in turn may cause, at the individual or (more worrisome) at the plurality level boredom with food choices (e.g., nutrition, taste) and/or food form factors (shape, size, texture, smell, etc.). Such food boredom may in turn result in decreased consumption, a reduction in nutritional intake, and even mental and/or physical distress and even illness. Exasperating this issue is the difficulty in formulating potential effective actions, such as changing meal types or even seasonings. Taco Tuesday might be a fun meal night, but not to people who despise Mexican food or just cannot tolerate cilantro. Further, in resource scarce communities you can't just “throw” a variety of food at the problem, confident there will be something for everyone. If you have Taco Tuesday, you will likely need to know within 2 or 3 tacos not only exactly how many will be eaten, but who will have eaten them, and have contingencies specifically ready for the others, perhaps without even having received direction from those non-Taco eaters about what they would want instead (as having to order each and every meal every day is in itself boring; people like to be surprised if they like the surprise). The ability to detect such potential Boredom may thus be a critical trigger detection requirement within the community. Bored workers become unmotivated workers. Or, put differently, it is the rare trigger that does not activate several other related triggers, such as being Bored triggering Depression triggering Loneliness triggering Social Interaction issues triggering work problems. A single problem community member can have a major impact on the specialized community as a whole given those communities unusual attributes (mentioned earlier). See, for example, the diagram shown in FIG. 19 depicting various examples of triggers (e.g., drinking triggers, etc.) and how the triggers may be related or interconnected such that one or more trigger(s) may activate one or more related trigger(s).

There are a variety of ways to detect, and in turn prevent/preempt/correct such food boredom. For detection, monitoring the intake of every consumable at the individual and plurality level may detect early drop-offs in consumption. As mentioned, this may not occur, or just occur, at the nutritional level, but at the form factor level. For example, plant-based foods may be “packaged” in a variety of ways, nut just as vegetable-type side dishes but in main meal, meat-type forms such as hamburger patties or hot dog/sausage link-type forms. A drop off in consumption a particular form (e.g., hamburger-type patty) may be an early indicator of Food Boredom. Similarly, a drop off in food consumption (again, at the individual or plurality of individuals) of food prepared with a certain oil (e.g., olive oil) or spice (e.g., paprika) could be an early indicator of Food Boredom and even more generally Boredom in a more “intensive” respect. Further, it may be critical to capture all associated contextual factors associated with the consumption.

Biometrics, particularly the eyes or other bio-cues, may be relied upon for quickly characterizing someone's mental state and awareness. For example, eye rolling by someone having just received leftovers for a fourth night in a row may indicate food boredom or dissatisfaction. Or a smile or other bio-cue may indicate food satisfaction at being served a new meal on the fifth night, etc.

The nature of monitoring for triggers such as (food) Boredom will be in many dimensions, not only for example just counting how much meat is left over from a meal (individual or multiple entities). The ability for example to monitor link versus patty (for sausages) consumption for the same consumable might be needed, and even the ability to measure how long it takes individual(s) to consume something as compared to his/her historical speed-of-eating. Even the ability to monitor/measure the decision process going into selecting patty versus link, such as how long is spent in reviewing the options before selecting (and how many of each are selected), could be an important measurement(s) similar to today's measuring how long a person stays on a screen before clicking to the next page, or how long the person tunes into a TV channel or movie before switching stations/movies. From a consumption standpoint, being able to distinguish any changes in consumption patterns as they related to context may also be important in determining root causes the “why”—of triggers, or even accurately diagnosing the triggers themselves. If for example, there is no drop off in patty consumption when food is offered in a buffet style and/or in environments where there are multiple people present, versus when food is individually ordered or selected, could be an indicator that perhaps a form of Loneliness is the trigger at issue, versus (food) Boredom. In turn, the pre-emptive “fix” for an emerging Loneliness issue (more social gatherings and recreation) would be very different than that for Food Boredom (offering more menu opportunities, retiring patties for 2 months, etc.).

Another exemplary embodiment, or really set of embodiments, of this disclosure is the need for, and ability to measure, assess, and diagnose the mental state of multiple entities within the community plurality of individuals, various forms of groups or pools, key demographics, etc. While an individual's health particularly mental health will of course be important, it will be especially critical to detect key issues/triggers that are occurring, or have the potential for occurring, in any significant part of the community, that have the potential for community-wide “contagion” and, in turn, may require a major drain on the (potentially very) limited resources to “treat”/address the trigger(s) in any sort of significant volume on individuals and/or ability to diminish the trigger at its community root.

Using the food Boredom as an example, the ability to notice any sort of plurality/multiple individual drop-off in interest in a certain food and/or food form factor could be far more impactful than any given individual's such boredom, even to the point of modifying production plans and/or various community contexts (how meals are offered/presented, mixture with other foods, amount of time before foods are replaced on the menu, etc.). Indeed, food Boredom, while perhaps a valid concern in itself, is potentially a much more important trigger as a leading indicator for multiple individuals/group-level discontentment, Boredom, Loneliness, etc. It could, when combined with other measurements/data, be a leading indicator of an entirely different trigger/root cause of discontentment, such as job or authority dissatisfaction, serving as kind of subconscious hunger strike for example, or even indicative of a group Depression.

Again, the potential existence of any sort of multiple individual/group trigger could have serious consequences for the specialized community as a whole, with its ability to “spread” far beyond the individual level, combined with the limited ability to “treat” the trigger(s) at any sort of scale. Thus, the ability to detect such triggers, diagnose root cause(s), and develop resource-optimized remedies/actions at the multi-entity level will be critical to the well-being and even survival of the community.

4) Nutrient Monitoring and/or Reclamation Systems

A key part of specialty farming/production and/or agriculture systems will be in reclaiming consumable by-products after they have been consumed and digested to the extent possible by the digestive systems of the entities consuming them. This will generally take the form of manure and/or urine-relate by-products. Even the gases produced from food or animals (e.g., cow methane gas) could have particular value as nutrients or have an impact to social cohesion as it relates to community social issues (climate, etc.). As farmers have known for many centuries, such by-products have utility in growing new consumables. In many specialty environments, with limited or no naturally occurring sources, such by-products will be a key source of nutrients, but with various costs involved, from unpleasant labor to energy consumption to scarce storage and production space (e.g., composting) to safety (e.g., not contaminating water sources/storage) to even social issues.

As such, the ability to reclaim these nutrients will be a key part of any specialty agricultural community. Specialized restrooms or similar will need to be utilized that collect, process, and distribute such by-products, and measure their volume, type, and nutritional quality throughout the reclamation process. Further, for at least some individuals with especially complex or difficult consumable (intestinal) requirements, their by-products will need to be examined on a regular basis to perform various tests to detect if there is a change in their body's digestive capabilities and/or detect early signs of deterioration or other negative signs of a potential physical and/or mental issue. If so, this could trigger a change in consumable requirements as well increase the volume and types of other monitoring of the individual as part of confirming such issues etc. More broadly, if human waste is an important input into a growing process, the amount and makeup of such “output” in general will need to be known and even predicted, and any deficiencies/inaccuracies in that output and associated allocation in growing/production cycles will need to be incorporated into overall agricultural planning and operations.

A related set of monitoring that will be required as part of specialty farming will include monitoring not only individual and collective waste by-products from individuals living there, but also calculating and monitoring the amount of nutrients that might be leaving the specialty site. For example, if a large party/feast occurs on the last night of a large lunar tourist trip, the tourists may depart with valuable nutrients ingested but not yet eliminated (e.g., undigested food, waste, etc.) from their bodies. In exemplary embodiments, the system is configured to be operable for anticipating and monitoring the nutritional value of the food to make up for it, or potentially even charging an extra “export” fee if a party takes too many nutrients without proper compensation. Indeed, as part of a “scoring” system disclosed herein some people may be awarded a certain amount/type of food (or other resource-type, like housing) “credits” that they can only use on certain consumables, saving other consumables for more “valuable” community members (e.g., those not leaving soon or otherwise valued differently). Such credits may tie to financial cost of the resource, its social impact, nutritional requirements individually or broadly, or some other variables(s).

### 5) “Base-Wide” or Colony-Wide Monitoring

Exemplary embodiments may include various forms of privacy elements, methods, and/or systems associated with or building off the nutrient monitoring and/or reclamation system disclosed above. In such exemplary embodiments, a software system may be used inside bases to help biological officers or life support managers at least guesstimate the various nutritional loads. This may be valuable in places where there is little to no naturally occurring nutrient sources such as the Moon but also on Mars, on spacecraft, and other off-Earth areas where nutritional collapse is a real possibility. These capabilities would likely be tightly tied to water, energy, and human by-product systems to ensure a full “closed” view of how a store of nutrition is utilized, and any leaks/inefficiencies detected and rectified. Given the highly personal and complex data and analytical needs, digital agents/machine learning/AI will be needed to both collect and analyze the large volumes of data needed (at the individual and aggregate level), and also include to the extent feasible given the context—capabilities to protect the privacy of the individual. Further complicating a scoring system may well depend on how the community's economy is based, e.g., capitalistic, communal, barter, etc. Indeed, a blockchain/crypto-based system might be combined with a community member value scoring system in helping to allocate certain resources, with certain crypto only being able to be used for certain goods or services, and/or only for certain contexts, or even only between certain individuals. Harsh perhaps, but specialized communities are by their nature highly susceptible to scarcity scenarios, and there needs to be flexibility and creativity in managing such scenarios with minimum amount of unpleasantness while the crisis is worked through.

6) Systems View Changes and Expansion with Learnings

Exemplary embodiments are configured to provide a highly flexible, learning-intensive larger system-level view of monitoring and soil and plant management with extensibility all harnessed together into software and/or human-managed systems. The ability to detect, and immediately respond to, changing production and consumption requirements, including modeling and understanding exactly (to the extent possible) how a change in one need or requirement will “ripple through” the entire production and consumption ecosystem, will need to be funneled into the growing processes and systems fast and dynamically. As learnings increase and are refined/improve, so will those individual leanings say for an individual module or portfolio be leveraged across other modules and portfolios, as well as scaled to impact the community as broadly as practical.

One way of doing the above is through the concept of the community's individuals having a “digital twin” that seeks to mirror, to the extent possible and relevant (and according to privacy constraints) the behavior and associated contributions of the individual. Through digital agents/machine learning/AI, such a system will model past and current individual (and/or group/pool) behavior. This could be used for the scoring purposes described earlier and/or to predict for various community scenarios and associated contexts, how the person(s) will behave, what the likely outcomes will be, and what adjustments may be necessary to improve the community's outcome in such scenarios. Given the potential for issues, crises, and/or full blown disasters in a closed community, such behavior/context-based scenario planning could be critical for the long-term health and prosperity of any such community.

7) Use of AI, Machine Learning, Etc. To Help Monitor, Control, and Improve Outcomes

Exemplary embodiments are configured to be operable for applying artificial intelligence (AI), machine learning, other techniques, and technologies to anticipate, monitor, control, and improve outcomes in specialty farming/production and agriculture. In this regard, exemplary embodiments for such methods and systems may include one or more features identical and/or similar to the AI and/or machine learning features disclosed in U.S. Patent Application Publication US2021/0202067, which is incorporated herein by reference. Accordingly, exemplary embodiments of the methods and technologies for managing specialty farming/production and/or agriculture systems may build upon and incorporate herein by reference one or more of the technologies and features disclosed in U.S. Patent Application Publication US2021/0202067. More broadly, the ability to model growing/production and consumption systems of all types (module based, portfolio based, individual based, pool based, demographic based, geographical based, and hybrids and combinations) through the use of Artificial Intelligence, Machine Learning, and the like will be essential, both to develop key learnings as well as how to leverage/scale the learnings more broadly while minimizing the impact at the micro-flexibility level.

Among various techniques that are envisioned is the use of Quantum Mechanics-related modeling, particularly its ability to model in incredible detail various cause and effects involving high volumes of dynamic behavior and contextual variables changing very rapidly.

8) Use of Software-Based Farm Labor System including Rewards and Incentivization

In exemplary embodiments, inputs in various other systems are used to derive work prioritization and scheduling, and assess its impact on flexibility and scalability, utilizing rewards and incentives as motivating tools and overall management business model. For example, both greenhouse A and greenhouse C may both need increased nitrogen, with the different greenhouses having different products/product modules serving different consumption pools as an example of the nature of closed communities continually having to make “competition”-like decisions in how resources are allocated and utilized. Sources of nitrogen in the overall community may be limited for example, such as ranging from uneaten/spoiled food (a rarity with a high disincentive all its own that needs to be incorporated into a rewards/scoring system, e.g., people who waste food will need to be in effect punished in some way, and associated measurement systems needing to appropriately capture what was electively “wasted” versus it having to be discarded due to some other factors not controllable to the person) to use of human and animal waste products. In situations where these sources are scarce in both greenhouses needing-to-add windows, incentives of various sources will be needed to motivate the right decisions regarding resources (and the labor to apply them) being used. This may also include providing incentives or bounties in work management and help to tie them together in multiple, sometimes at-odds views utilizing component resources as critical management inputs. Modules or pool portfolios could reflect specific crops for example, conditions, nutritional needs, and/or person or person groups, as well as other factors such as geographical and/or contextual variances in growing and/or consumption environments. While the above is focused on human labor, using augmented human and/or full robotic labor could also apply, particularly is such resources were in themselves short supply, and/or limited in some way in terms of functionality, capacity, or even contexts.

By way of example, exemplary embodiments of the specialty farming/production and/or agriculture systems disclosed herein may include one or more of aspects or features of the dynamic and adaptive systems and methods for rewarding and/or disincentivizing behaviors disclosed in U.S. Patent Application Publication US2021/0202067, which is incorporated herein by reference. In exemplary embodiments, the methods and technologies for specialty farming/production and/or agriculture systems build upon and incorporate herein by reference one or more of the technologies and features disclosed in U.S. Patent Application Publication US2021/0202067.

### 9) Regolith Processing Systems

As recognized herein, there is a lack or void of knowledge regarding the kinds of tools that might be valuable in specialty farming/production and agriculture and the variations of such tools between different specialty communities. For example, how would a shovel, tractor, combine, other farming tool, implement or machine be configured for farming on the Moon, versus “traditional” on-Earth usage or even other On-Earth specialty communities? As also recognized herein, farming implements may be replaced by multipurpose smaller robots. Accordingly, exemplary embodiments include one or more tools, implements, machines, robots, networks, software, combinations thereof, etc. configured for use when farming off-Earth, e.g., on the Moon, etc.

Further, there is a significant likelihood that tools themselves will need to evolve within various specialty communities as more data is collected on their usage in different contexts. As a non-limiting example, just using a small traditional hand shovel in low-gravity situations may be problematic, as the digging motion of a human has a tendency to throw dirt into the air a minor nuisance on-Earth but potentially a major one in lower gravities. As such, a special screen attached to the shovel may be needed, and/or the context may need to be changed, e.g., increasing the gravity associated with a module during planting, weeding, or harvesting cycles. An embodiment of this disclosure includes the processing and transformation of regolith using mechanical, chemical, or biological systems—for example biological application of microbes for reduction of oxides to create new mineral formations to weather the soil. Using mechanical stress such as crushing or tumbling to change the shape and structure of the regolith grains, the inoculation of microorganisms or larger organisms like earthworms, with the focus on optimization of soil health characteristics such as Ph, available iron, NPK bioavailability etc. Use of chemical weathering such as using water solutions with other chemicals, acids, alkalis, and other chemical amendments to alter the chemistry. Additional possibilities include using novel and unprecedented on Earth such as using electricity to alter the regolith. Additionally using the byproducts of other regolith processing systems such as but not limited to electrolysis for metal and oxygen production, slag from various other, water ice (and other ices) mining, and any other industries that may result in leftover piles. Besides in itself being a resource-improving capability, its usage could (and likely would) require non-traditional tools and processes to adapt/be adapted to various new/modified specialty community contexts, as could how humans behave (differently) in various contexts using those various techniques/systems/methods such as involving regolith.

10) Incorporation of Tailings Piles from Other Systems

Generally, tailings refer to materials (e.g., rock waste, other materials, etc.) leftover from a mining or industrial operation after extraction of the valuable portion (e.g., extracted mineral, etc.). In exemplary embodiments, the leftover raw material or semi-processed material is used and serves as a valuable input into the specialty farming/production and/or agriculture systems. In such exemplary embodiments, the reuse of the tailings helps reduce waste. And the tailings are already in a form that is relatively easily maneuverable due to the initial extraction and possible other completed operations such as particle size sorting, etc. Exemplary embodiments may include Application Programming Interfaces (APIs) (e.g., agricultural interface(s), etc.) incorporated into a mining, industrial (e.g., metal production/manufacturing), and/or extraction systems to thereby enable reuse of tailings, including the ability to produce oxygen from such tailings. Other possible industries which may result in tailing piles or other similar leftover materials include production of ceramics, chemical production, metal production, wastewater recycling, sewage recycling, oxygen production, ice mining and refining, and any other industries that may be occurring within or nearby a specialty community.

### 11) Resource Reclamation Systems

As disclosed above, exemplary embodiments may be configured to be operable for monitoring and/or reclaiming nutrient loss. Additionally or alternatively, exemplary embodiments may include systems and methods focused on composting and reclamation of nutrients (e.g., reclaiming as many nutrients as possible, etc.) particularly major elements like Nitrogen, Phosphorus, and/or Potassium but also trace elements such as but not limited to like zinc, iron, calcium and any other micronutrient that can be reclaimed. Such exemplary embodiments may include composters, biodigesters, other organic extractors, combinations thereof, etc. Exemplary embodiments may include a software system operable for monitoring and potentially rewarding or incentivizing proper composting of biological waste as well as the proper tracking, management and reclamation of any waste. For example, an exemplary embodiment of a system may be configured to monitor space tourist(s) to confirm proper compositing of biological waste(s) and to award a small credit in compensation for helping the nutrition and biological health of the colony. In such exemplary embodiments, the system may include one or more of aspects or features of the dynamic and adaptive systems and methods for rewarding and/or disincentivizing behaviors disclosed in U.S. Patent Application Publication US2021/0202067, which is incorporated herein by reference.

### 12) Disease Detection and Resolution System(s)

Returning to the monitoring aspects, exemplary embodiments may be configured or extended beyond simply monitoring for nutritional value for both consumers and the plants themselves. Exemplary embodiments may be configured to be operable for monitoring for and detecting potential disease(s) including novel off-Earth based diseases that might occur within the plants, etc. In such exemplary embodiments, the disease monitoring and detection may include using optical, air, spectral any other type of sensor (e.g., Internet of Things (IoT) sensors, etc.), combinations thereof, etc. After detecting a disease(s), exemplary embodiments may be configured to help resolve the adverse situation (e.g., establishing a quarantine zone, recommend therapeutic or other treatment, etc.). By way of example, exemplary embodiments may include one or more features of a quarantine-related exemplary embodiment as disclosed in U.S. Pat. No. 11,412,353 and U.S. Patent Application Publication US2022/0386080, which are incorporated herein by reference.

Disease detection and resolution embodiments include integration with other types of embodiments included, including detection/responses to changing human behaviors and preferences, wastage, and even rewards/incentive systems, in addition to changing reallocation of resources to increase certain medicinal herbs as a result to a predicted ailment increase in a person(s). For example, monitoring/detecting changing tastes/preferences could be an early detection mechanism for a certain ailment (with a recent example being a loss of taste associated with COVID). In closed communities, with a constrained food supply chain, such change in taste or food cravings could be especially material as a disease early-warning system, or a nutritional deficiency detection mechanism, or even an airborne microbe detection mechanism, particularly if such conditions are detected in a rapidly growing set of persons over a short period of time. Put another way, a change in behavior (particularly in certain contexts) in specialized communities may serve as an effective early warning system for a variety of issues far more than is possible in traditional on-Earth communities, with its extremely variable, generally uncontrolled contexts.

### 13) Nutrient Monitoring (e.g., Brix Reading, Etc.) for Improving Food

Exemplary embodiments may be configured to be operable for monitoring and grading of the resulting produce off-Earth (or on-Earth specialty communities) as managing diet of a person (particularly off-Earth) will be an essential life support function particularly in the early days off-Earth colonization. Advantageously, such exemplary embodiments may therefore provide valuable information or knowledge as to exactly what the nutrition needed/desired is for an individual (e.g., fruit(s), vegetable(s), combination thereof, etc.) to help address or alleviate various health concerns, productivity/motivation, or even just general mental health. In turn, this nutrition, at the individual, group, pool, or other combination of individuals, can have a direct or indirect impact on the resources needed for the nutrition consumable, such as types, amounts, and timing of organic versus chemical fertilizers, if, how and what type of pesticides, etc., as well as dictating or influencing the amount of light/radiation exposure, water, humidity, temperatures, etc.

As such, such nutrient-related data will be key inputs/aspects of many of the above embodiments, and such data will need to be highly tightly tied to individual physical and mental data collection mechanisms, using a variety of wearable, attached, and/or embedded sensor mechanisms in the individual to detect and measure various biomarkers, as well as various context detection and measurement mechanisms (such as Internet of Things sensor deployment through a room and “things” in the room) to appropriately calibrate/normalize etc. the individual physical/mental/behavioral data with the conditions in which that data was collected.

14) Incorporation into Health Monitoring Systems

Exemplary embodiments may be incorporated into health monitoring systems similar to those of astronauts, e.g., to improve diet as well as suggest supplements to improve health and fitness. Moreover, thus systems could be at the multiple individual level, and could focus generally on the applicable individual(s) and/or the context they are in, static and/or dynamic (e.g., traveling through space in a ship; fluctuating gravity, radiation levels, etc.). For example, in addition to sensors on an individual astronaut on a space station, sensors could be deployed in working areas where multiple individuals can be, including changing numbers and particulars of who is in a particular area, when, doing what, why, and how. Such exemplary embodiments may build upon the nutritional, disease, and other types of monitoring described above. For example, the system may be configured to use or include health-based IoT to monitor overall health and provide suggestions to improve diet using both human and AI inputs. The system may be configurable and/or adjustable to accommodate for variants such as long-term stays and short-term stays as well as tourists who may each have different concerns for both health and privacy.

Monitoring could be various external and internal type monitoring including camera observation, use of AI, machine learning, predictive models, or transmission to professionals for monitoring. Cues such a blood pressure, eyes, breathing patterns, sleep patterns, general behavioral cues (such as deviations, lethargy or starting to visit unusual patterns) and other monitoring. Models can also include interactions with other agents within the system including computer systems, animals, plants and other humans.

Another aspect of the disclosure is in the health monitoring of groups/pools of living entities within the specialized community. Note that such groups can be multiple, one, even no such actual entities. In effect empty groups can be used for example (but not limited to) certain kinds of health conditions that did not exist when the community was established, but could develop over time and need to be monitored for accordingly. Scurvy on old-fashioned sailing ships taking long voyages would be an example where the group would be empty at the start of the voyage, but several months later could be half the ship population or more. In fact, the monitoring of/for potential ailments as a group could be critical in specialty communities, as the potential for certain kinds of mental and/or physical ailments could be especially “contagious” in such environments, even if the ailment is not a type of contagion spread by bodily contact or other such mechanisms. The saying “panic is contagious” is an example of a non-physically-spread ailment. Anxiety, Depression, and other mentally focused ailments are examples of such. Being around a depressed or anxious person in a closed community, particularly over time, could well spread to others who might not otherwise feel in that respect. Thus, the ability to monitor for ailments not yet existing or in low levels and/or “harmless” states will be critical, as well as preemptively addressing any appearance of the ailment in the broader population.

### 15) Value-Added Processing

In exemplary embodiments, food-related value-added processing (e.g., creation of sauerkraut, grinding flour, etc.) is included or integrated as part of farm and food management. For example, exemplary embodiments may include a flour grinder configured for use in lower gravity or microgravity, a machine to make food incorporating “specialty” settings such as adjustments for atmospheric density, gravity, etc. or other kitchen items configured for use in specialty environments (“utensils”).

Such utensils, besides being “Smart” (e.g., ability to be configured and remotely controlled), will have an array of sensors and connectivity to sensors to adjust for the purpose it is being used for (e.g., specialty processing for an individual or pool of individuals with specialty needs), volume of production, and the context in which it is being used (where in the food production lifecycle the utensil is being used), environmental conditions (pressure, temperature, gravity, humidity), and duration of use, for example.

The above will be utilized not just in preparing foods etc. for to-be-immediately consumed needs, but for pre-emptive purposes. As noted above, a key part of the disclosure (including the patent applications and patents incorporated herein by reference) is in the prevention/pre-emption of negative events, occurrences, situations, and so forth e.g., negative contexts and/or behaviors that lead to trigger thresholds being approached/reached/exceeded. For example, as noted, the disclosure includes capabilities for monitoring for rising anxiety levels and predicting/forecasting when an individual(s)/pool(s) of individuals may reach a threshold in their anxiety levels that will need an increased amount of an anxiety-reducing type of food, additive, herb, medicine, etc. Since there will likely be heavy demand for food preparation utensils (particularly those that can accommodate specialty community needs), as well as support needs (labor, key ingredients, power needs, water requirements, etc.), these will need to be reserved/scheduled far in advance, particularly for food, etc. that have a short shelf life and cannot be produced far in advance of need. In effect, pre-planned emergency preparation may be needed anticipating the use of a myriad of scarce resources in critical contexts.

### 16) Incorporation of Animal Management Systems

In exemplary embodiments, animal-related systems are incorporated for improving both soil and food options (e.g., animal to field feedback loop, animal to plate feedback improvement, etc.). For the same or similar reasons provided above, animals may be incorporated for not only improving soil health, but also for animal health reasons, such as to increase nutritional value of the animals and other uses the animals may have off-Earth. Further, animal “management” could include not all the human-related embodiments described above (e.g., related to nutrition, health management (both physical and mental), disease, etc.), but also the embodiments related to food supply chain production, e.g., growing, harvesting, etc. In fact, given that to date there is little to no data on animal management/production in any sort of specialized community, animal management systems in such communities may well be good “guinea pigs” as far as how human-related systems in such communities will “behave” and generate similar issues. Put another way, monitoring and responding to issues in the animal management systems may need to be linked to the human systems, as the issues they encounter may be similar.

17) Monitoring and Improving Health of Animals off-Earth

As disclosed above, exemplary embodiments may be configured with and/or include health monitoring. In some exemplary embodiments, the health monitoring is extended to animals (e.g., specialty animal husbandry, off-Earth zoos, etc.) for monitoring and improving health of animals off-Earth and even on Earth, e.g., endangered species.

### 18) Systems for Quality Control and Monitoring

Exemplary embodiments may be configured with and/or include systems for quality control and monitoring for improving the quality of goods to return to Earth, including preventing/preempting the counterfeiting of goods produced in specialty communities (thus likely to be considered more valuable due to scarcity, status, unique qualities, and/or superior quality considerations. In such exemplary embodiments, IoT system(s) may be used for monitoring quality control of goods to return to (traditional) Earth or other specialized communities. For example, people on Earth may want Moon-made cheese, Moon-made whiskey or other consumable made on the Moon or elsewhere off-Earth or in Earth specialized communities (underwater farms, etc.). In such cases, such as off-EARTH, an exemplary system(s) disclosed herein may be configured to be operable for monitoring the off-Earth supply chain for purposes of quality control, packaging, and/or distribution. It is possible, even likely, that the end-market price for such specialty community-made consumables may be a huge multiple of the traditionally made equivalent—possibly thousands of times or higher. Thus, a pound of “moon stilton cheese” may be $100,000, even a $1,000,000, vs $10 for traditionally made cheese. Or a cask of “Moon Brandy/Cognac” could fetch $10,000,000 or more.

Such price differentials will of course run the very high risk of counterfeit and fakes, and/or causing a high risk of theft and other crimes to obtain it. Thus, quality assurance-focused, step-by-step certification, counterfeit-prevention security will need to be part of every aspect of these “Moon Brandy/Cognac” consumables. Not only would all manners of data collection, verification, and security be employed, the use of blockchain and cryptocurrencies and derivatives, such as NFT (Non-Fungible Tokens) will be needed at key points in the production cycle/food supply chain to ensure (and prove) that critical ingredients and/or steps in the production chain have been provided/done in a way that only could have been done in that specialty community, at that time and place, using specialty community resources (of all sorts), for that purpose (making of food product XYZ or manufactured item ABC. These NFT's in effect will become part of the “Moon-Cognac” end product, and without them the Moon Cognac will deemed to be fake, or stolen. In fact, specialty crypto-currency may be created for that production lot of Moon Cognac, with the amount in circulation aligning precisely with the amount of moon cheese produced, Indeed, it may be that the only way to purchase/own Moon Cognac would be through the acquisition of the moon cheese crypto, such as 1 MCT (Moon Cognac token) being redeemable for 1 ounce of Moon Cognac. Such crypto value could be open ended/not expiring, and redeemable much like a U.S. silver certificate dollar bill could historically be redeemed directly for that amount of silver. Or, if a consumable had an expiration (such as a Moon Cheese, the Moon Cheese NFT could be bought/sold on a futures or options-type market, gradually changing value until it became worthless in alignment with the Moon Cheese becoming inedible.

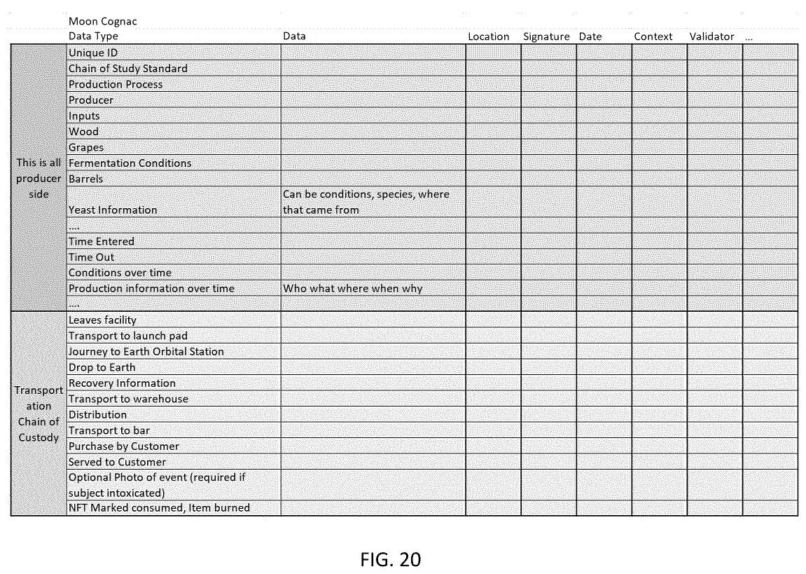

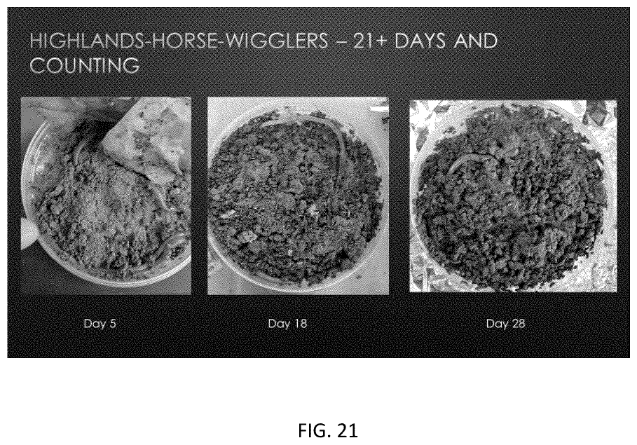

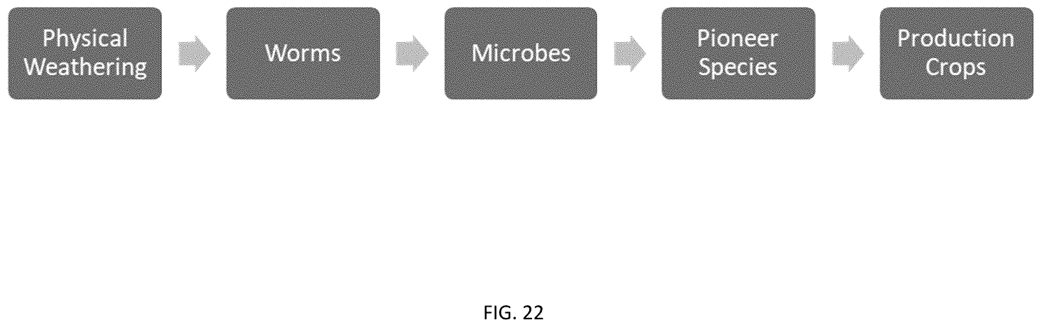

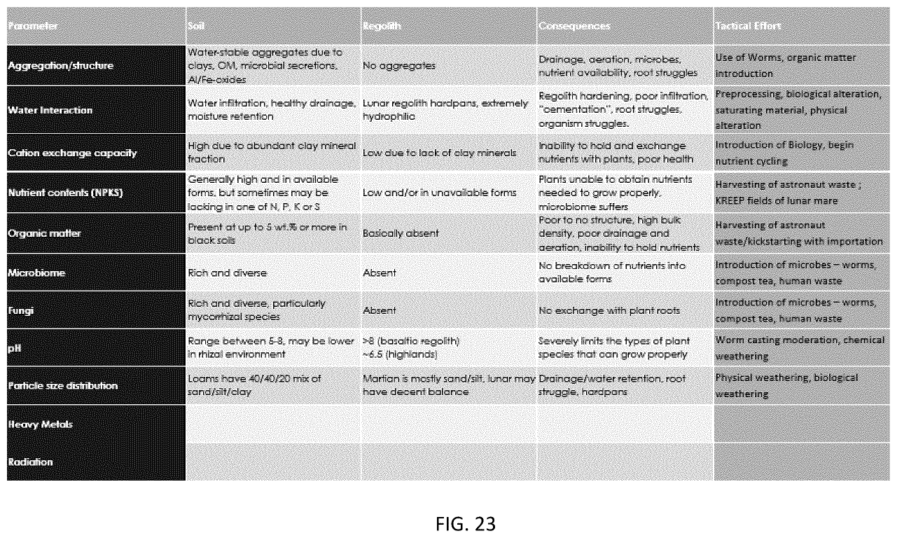

In such case(s), an exemplary system(s) disclosed herein may be configured to be operable for monitoring the specialty community consumable for purposes of quality control, packaging management, and/or distribution control, as well as associated benefits of counterfeiting prevention and asset verification, validation, and valuation. Various forms of evidence in the form of blockchain transactions will be established during the course of production with potential value being added literally using cryptocurrency during key steps of production, such as illustrated in FIG. 20.

As the table in FIG. 20 illustrates, every aspect of the good's (such as a bottle of Moon Cognac) would be meticulously tracked and recorded, not only on the production side but once it leaves the Moon (in this example) and enters the marketplace. Existing counterfeit systems, to the extent they exist, focus on the end product and perhaps purchaser history. In specialty community production environments, part of the value is knowing that EVERY part of the item production was done within the specialized community, and indeed may have actual value (in the form of crypto) that literally values work/production in progress, not just end product milestones.

### 19) Human and Robotic Tenders Management

Exemplary embodiments may be configured with and/or include a project and work management system that is operable for harmonizing or providing harmony between human and robotic actors. This will include inclusion of machine learning/AI digital agents working on behalf of a given human (or even attempting to minor that human's behavior in certain contexts), and those agents working in coordination with robots (or the robot's agent) as human/robot behaviors are coordinated for given tasks/behaviors in given contexts.

Achieving such coordination itself could require specialized integration/coordination agents, including calibration capabilities that appropriate modify a robot's action to recognize that such actions are likely to be faster, and possibly more impactful/productive, than an equivalent human's action, in certain contexts. Alternatively, for contexts that are more service oriented (e.g., robots are service providers to humans), the integration/coordination becomes more of a human providing the objective and assigning the task and expectations associated with the task, and the robot acting on the task to meet the human's expectations.

### 20) Monitoring and Control of Robotic Equipment

Exemplary embodiments may be configured to be operable for monitoring and controlling robotic equipment including tasking and maintenance. Such exemplary embodiments may be configured for monitoring and maintenance of new off-Earth/specialty farm technology and may include on the software side using IoT sensors to help keep things working well as well as logistically manage the equipment in tracking what equipment was used, when, where, by who, why, and how. As recognized herein, this may be more difficult to do in specialized communities (with different reference systems than on traditional Earth) and that actually tracking usage and associated maintenance needed may be very different for such equipment due to the potentially dramatically different use cases of the equipment (utilized in a wide range of gravity conditions) as well as the contexts within which the equipment was used (e.g., sometimes used by humans, sometimes by robots) and/or different resource constraints in how and how often they are to be maintained (e.g., limited lubrication oil).

### 21) Broad Scale Systems, Large Acreage, Domed, or Underground

In exemplary embodiments, the methods and technologies disclosed herein are applicable to small greenhouses as well as broad scale agricultural sites, e.g., on the scale of several acres, hundreds of acres or more, etc. It is anticipated for various reasons, that there will be a substantial increase in various specialty communities with their own food supply chains, as space colonization begins and various “prepper” communities become more prevalent, just to name two. The resources for these varying purpose communities will also vary, As will the associated tools. The tools for the associated small greenhouses and other small agricultural sites, may differ from the tools for the large scale agricultural sites. Variations on historical “dumb” farmer tools may need to be used in smaller environments, with modifications (hand shovel with special screen for lower gravity for example), with more robot use and more ability to dynamically (and automatically) reallocate resources to different food production immediately upon detecting the need for such reallocation. Further, by their nature smaller communities with smaller areas used for farming will have much “wiggle-room” for food production miscalculations, making the mechanisms described herein that much more important. Conversely, while off-Earth colonization will likely become large scale in many ways, there are obvious inherent limitations (e.g., naturally non-productive soil, no water) to associated large scale agriculture, also necessitating the mechanisms described herein. Domed, underground, or underwater areas will tend towards the smaller size, but potentially have advantages in terms of being able to control at least some contextual elements needed for farming, such as climate control within domed areas.

### 22) Intensive Systems for Hydroponics or Intensive Gardening

Exemplary embodiments may be configured for using hydroponics or intensive stacking in specialty farming, which may include or incorporate IoT elements, e.g., IoT sensors, mesh networks, etc. Further, such methods are much less studied on Earth (with its mostly horizontal, earth-based farming methods), and thus the systems for deploying them in specialty environments, especially off-Earth, will likely be an almost greenfield environment for learning how to do such specialty farming. Many traditional on Earth methods and tools will by itself will likely be transformed moving in effect from a single 3 dimensional context (plot of land, planting one seed to certain depth, with only one plant growing vertically), to stacks of such plots, e.g., using vertical space/height (a Z dimension) to plant more within the same amount of X and Y real estate. This kind of “true” 3 dimensional farming will necessitate many of the mechanisms described herein. For example, just watering a true 3D plot with several plants in small area will necessitate different irrigation systems than used for “simple” 3D planting (e.g., with just one plant in a vertical space). Being able to measure, apply, and water several plants will require intensive sensors/sensor arrays and real-time network measurements to ensure that all the plants in a vertical space will be evenly watered, and as importantly no water goes to waste.

### 23) Inclusion of Regenerative Techniques in Transforming Soil

In general, regenerative agriculture tends to focus on turning regolith into soil. Exemplary embodiments include or relate to transformation of regolith with worms and/or biologics (e.g., bacteria, fungus, other small soil critters, etc.). For example, a system may be configured to be operable for monitoring the regolith to soil transformation, e.g., via worms and/or biologics, etc. The system may also be configured to be operable for identifying where it would be beneficial to add various worms and/or biologics, etc. Monitoring and tracking such specialized resources (e.g., worms, etc.) may itself need specialized processing and systems, which will in turn need to be integrated with other system and method embodiments disclosed herein. Since in specialized communities resources are scarce, processes are untested, growing/production contexts are new, and successful growing/production of food may depend on even the smallest factor being right, no resource no matter how small is too small or insignificant to be monitored, tracked, and integrated into the resources used in the community's supply chain. Indeed, it may be that factors deemed unimportant in traditional earth farming (types of worms and ways to stimulate them) become critical factors in growing food off Earth or under other specialty conditions. In such a case example, it may be necessary to tag or otherwise identify individual worms, where they move, why, how etc., and alter conditions to make some portion of the (finite) amount of (possibly specialized) worms “reallocate themselves” from growing one food item to another food item as part of a reprioritization/reallocation of resources from the first foot item to the second food item.

### 24) Use of Information Management System

Exemplary embodiments may be configured to and/or include an information management system including blockchain systems and/or non-blockchain systems. The use of such systems may be standalone (e.g., controlled fully within the specialized community), using existing such systems in/on traditional Earth, or a combination of the two. More broadly, there will be numerous architectures employed in such systems, including but not limited to client-server, IoT-type mesh or Zigbee networks, Edge-computing, and centralized and/or fully distributed (e.g., highly localized control) systems. Use cases will vary widely, from collection of data from individuals via wearable sensors, to arrays for collecting detailed contextual data, to various control systems for managing crops, to machines capable of enabling massive machine learning/AI capabilities, to close ties with other community computer/networks.

### 25) Processing, Preserving, and Packaging

Processing, preserving, and packaging of food may include dehydration and nutrition management for ready-to-eat meals. Crops grown off-Earth or specialty on-Earth (e.g., in desert conditions) will likely be dehydrated to recover the water for immediate use and allow for longer term storage and compacting. These processes, combined with the nutrition management system disclosed herein, may be necessary for long term sustainability, as off-Earth outposts may need or require large food stocks in case transport of supplies from Earth are delayed for any reason, and the ability to reclaim water from any/all sources in circumstances where ice/water is extremely limited. In which case, the off-Earth post inhabitants would need to have a sufficiently large food (and water) stock to be able to either make a return trip to Earth or have enough time to replenish supplies locally at the off-Earth post.

As discussed in various places, triggers like Anger, Boredom, Money, Stress, and so on are those “things” which can, in turn, cause or serve as the primary catalyst for or otherwise “activate” certain (usually negative) behavior, such as an alcoholic being triggered to drink/relapse. While triggers can often be enough by themselves to “cause” or otherwise result in the (undesired) behavior, that is not always the case, particularly as many triggers are emotional in nature at least in part, and emotions tend to “blend into” or merge with other emotions (and thus triggers), and/or causing/triggering other emotions/triggers to occur, and/or vice versa. For example, Anxiety can cause/lead to/trigger Depression or vice versa. Boredom can cause/lead to/trigger Loneliness, which can, in turn, lead et al. to Depression. Kids (children) chaos can cause Noise which can result in Yelling, which can result in Anger. These are all “related triggers” to the original trigger that started the process. FIG. 19 illustrates how triggers can be “related.” The practical effect of a trigger activating related triggers is a kind of emotional/trigger “snowball” that rolls over the sufferer. Some of the snowballs “picked up” after the ball starts rolling may be relatively small, or as big if not bigger than the original snowball-starting trigger, just like in the physical snowball world. This makes preempting the original “upstream” snowball (e.g., the one highest on the mountain) to begin with so critical.

In an alcoholic example, it is not uncommon for alcoholics to have somewhere between 5 and 10 (or even more) very significant or major triggers, e.g., a trigger(s) that, in and of itself or by its very nature, can individually lead to a relapse. These significant/major triggers really make the person want to (or “have to”) drink, more often than not. They can make the person want to drink all by themselves, e.g., independently of anything else going on in the person's life. But they also can set off a number of “related” triggers. Using a straw-that-broke-the-camels-back metaphor, a significant/major trigger (and possibly more) can serve as the first 900 straws on the camel-relapse back that can stand 1000 until it breaks (relapses). One or more related triggers—for that person—might serve as the 101 straws that puts the camel over the top: breaks its back. And these triggers, and how much they “weigh”, can depend on the context, with different triggers having different straw “weights” in different contexts. Indeed, for a given person, one trigger might be significant/major and another related trigger relatively minor in impact; in other contexts the reverse might be true, or other triggers coming into the mix in varying degrees. In effect, for different people, and even for the same person, any/all of the triggers (and others) shown in FIG. 19 might be both significant/major triggers as well as related triggers for other significant/major triggers depending on the context and the person. This sea of interrelated triggers is a different concept than is “taught” in rehabilitation programs of all sorts. Even when traditional treatment programs go through some degree of trigger discussion, they nearly always “treat” triggers as “standalone”, e.g., the focus is on the effects that a trigger has on your drinking habits (in an alcoholic example) due to that trigger all by itself. At best (worst) such treatments might incorporate the concept of “dual-diagnosis” of Alcoholism and Depression for example, or Anxiety and Depression in a more general mental health treatment program.

Unfortunately, the pressures of daily life rarely line up in such single file fashion. Certain situations in a person's daily, historical, or ongoing life can “activate” or trigger other triggers. Again, these are referred to herein as related triggers. For example, Boredom can make you Lonely, which, in turn, might make you want to go out with friends who drink (causing direct or indirect Peer Pressure), which may take you to a place where Proximity and Smell of nearby alcohol has you drooling for a Taste of alcohol to help you Escape from other problems in your life. It is incredibly difficult to defend against alcohol in all of these simultaneous/near-simultaneous circumstances. There are hundreds, even thousands of possible combinations. And many of them, in relevance, degree, impact, and combination can vary in their “snowball building” by context, making treatment far more complex. And life is complex—what further makes determining/diagnosing/treatment so difficult is that many triggers often “attack” at the same time or nearly, and/or occur so often in conjunction with related triggers that it sometimes becomes impossible to sort out the different triggers involved, and what is a “cause” and what is an “effect”.

To add even more complexity, a person's defenses, such as an alcoholic's defenses, may be weaker for some triggers than others. Worse still the strength of the person's defense may vary depending on the hour of the day, day of the week, personal living environment at any given time, how their day at the job went, and so on. In total, this complexity of trigger/related trigger relationships and variability of how and when they attack makes it almost impossible to build a single defense that works against all of a person's vulnerabilities all of the time—a much more sophisticated set of systems, methods/processes, and mechanisms are needed—examples of which are disclosed herein.

By way of example, providing an incentive may be in effect also providing a form of disincentive, e.g., by doing X you are NOT doing Y. By rewarding not drinking, you are inherently penalizing drinking even if there is no overt penalty for drinking (just rewards for not drinking).

By way of example, a blockchain/crypto-based system might be combined with a community member value scoring system in helping to allocate certain resources, with certain crypto only being able to be used for certain goods or services, and/or only for certain contexts, or even only between certain individuals. Harsh perhaps, but specialized communities are by their nature highly susceptible to scarcity scenarios, and there needs to be flexibility and creativity in managing such scenarios with minimum amount of unpleasantness while the crisis is worked through.

With respect to scoring of the specialty community “contribution” (e.g. figuring out the absolute and/or relative value of a person's contribution), exemplary embodiments of the system may be adapted or configured to be operable to measure (dis)respect. It is theorized that a huge amount of today's young person murders are due to some variation of being, or being perceived to have been, “disrespected.” In exemplary embodiments, the system may be configured to be operable with a kind of (dis)respect score that could be rewarded (or penalized/punished) financially or in goods and services, perhaps young people would think twice before picking up a gun and ruining both their lives with violence. The system may be used, for example, in a crime-ridden neighborhood being fought over by a couple gangs, which crime-ridden neighborhood could be considered a specialty or specialized community. Indeed, detection of and tracking of gangs and preemption of gang activities before they deteriorate to negative activities will be a key community health activity. More broadly, the use of such potentially negative (as well as mitigating positive activity) will be a key part of new/improved forensics capabilities that will be needed by special communities and enabled by the abilities described above. Means, motive, and opportunity determinations will be greatly enhanced, particularly with respect to context detection and AI motive analysis.

In exemplary embodiments, the need for a wide range of monitoring/data collection, analysis, intelligent agents, predication engines, action development, and machine learning/feedback capabilities may be needed, applied to and integrated with the individual and multiple persons, to their living and working and leisure/playing environments, to who/what they interact with and how, and more broadly the ability to monitor and measure and predict and respond to an individual's potential impact on a community's production and consumption chains, utilizing a wide range of data collection and analysis mechanisms, including but not limited to wearable sensors, community (inside and out) sensor arrays, context determination/monitoring mechanisms, various forms of communications networks, and digital agent/machine learning/AI capabilities.

In exemplary embodiments, the system is configured to implement a digital twin(s) for an individual(s) of the community that seeks to mirror behavior and associated contributions of the individual to the community. One exemplary way of doing this through the concept of the community's individuals having a “digital twin” that seeks to mirror, to the extent possible and relevant (and according to privacy constraints) the behavior and associated contributions of the individual. Through digital agents/machine learning/AI, such as system will model past and current individual (and/or group/pool) behavior. This could be used for the scoring purposes described earlier, and/or to predict, for various community scenarios and associated contexts, how the person(s) will behave, what the likely outcomes will be, and what adjustments may be necessary to improve the community's outcome in such scenarios.

In exemplary embodiments, achieving coordination between human and robotic entities may rely upon specialized integration/coordination agents, including calibration capabilities that appropriate modify a robot's action to recognize that such actions are likely to be faster, and possibly more impactful/productive, than an equivalent human's action, in certain contexts. Alternatively, for contexts that are more service oriented (e.g., robots are service providers to humans), the integration/coordination becomes more of a human providing the objective and assigning the task and expectations associated with the task, and the robot acting on the task to meet the human's expectations.

In exemplary embodiments, a system is configured for managing specialty community agriculture and associated resources utilizing needs-based, context-based, and behavior-driven integrated production and consumption resource management capabilities and resource inputs. The system comprises a plurality of different devices, sensors, other systems, and/or communications network(s) configured to dynamically and flexibly manage modular planting, growing, treatment, harvesting, producing, acquiring, generating, distribution, storage, and/or consumption of consumable resources of a community(s). The system is configured to be operable for determining, assessing, analyzing, (re)allocating, and/or predicting future production and/or consumption resources to support future forecasted, predicted, and/or possible needs and associated behaviors and contexts of at least a plurality of humans within, expected to be within, and/or requirement to be within a human community population of the community(s).

The producing, acquiring, and generating of consumable resources of the community(s) may include things like mining, energy production, pumping water, and generating key resources, including imports into the community from outside. And consumption may also include use of a resource as an input into other production, such as salt as an additive to meals, or even waste production resulting from consumption used as fertilizers.

In exemplary embodiments, the system is configured for utilizing a plurality of digital agents that measure, monitor, track, assess and/or analyze human(s), equipment, and/or community(s) needs, contexts, behaviors, and associated resources, to develop, proffer, and/or implement recommendations associated with said needs, contexts, behaviors and/or associated resources using a scoring capability(s).

In exemplary embodiments, the resources include one or more agricultural inputs and outputs, life support, energy, water, soil and/or regolith, structural and construction, communication, transportation, healthcare, environmental control, scientific and research, security and safety, social and recreational, supply chain and logistics, crew and support, and/or sustainable, physical, electronic, mental health, and/or intangible resources needed to meet said needs. The needs-based capabilities include assessment of past, present, and future production and/or consumption needs and associated resources. The needs include needs of an entity comprising two or more of Physiological, Safety, Love/Relationships, Esteem, Actualization, Transcendence, setup, configuration, operating, maintenance, and/or retirement needs, with such needs being associated with one or more resources. The needs of a community include two or more of physical, mental health, stress, nutritional, occupational and skills, family, age and life stage, education and skill development, age and life stage, energy and water usage, financial, community engagement, environmental impact, emergency preparedness, transportation, cultural and social preferences, and resource feedback and reporting capabilities. The contexts include two or more of social, work, cultural, economic, family, health, environmental, political, technology, emergency or crisis, legal, religious or spiritual, psychological, and/or recreational contexts. The behaviors include one or more of prosocial, antisocial, altruistic, aggressive, cooperative, competitive, assertive passive, impulsive, risk-taking, procrastination, adaptive, maladaptive, empathetic, conformity, nonconformity, risk-adverse, risk-seeking, self-regulation, and/or help-seeking behaviors.

In exemplary embodiments, the system is configured such that the needs, contexts, behaviors, and associated resources are capable of being measured, determined, estimated, monitored, and/or tracked, with such capability(s) being able to determine, estimate, and/or generate a score or an input into a scoring system(s). The system is configured such that the scoring system(s) utilizes one or more of a quantitative, numerical qualitative, color coding, binary, ordinal ranking, textual feedback, graphical representation(s), heatmap(s), percentage-based, checklist, threshold-based, range-based, risk matrices, machine learning/AI, natural language processing, sensor alarms, health-based and/or behavioral analysis algorithm. The system is configured such that score(s) of the scoring system(s) serve as inputs and/or mechanisms into recommendation development and implementation of changes/modifications to needs, behaviors, contexts, and/or resource utilization, resource (re)allocation and associated processes, and/or dynamically and flexibly manage modular planting, growing, treatment, harvesting, producing, acquiring, generating, distribution, storage, and/or consumption of consumable resources of the community(s). The system is configured such that behavior recommendations are based on the context associated with behavior, an entity and/or community needs that need to be addressed by the individual, and on the potential for positive impact to the entity's personal and/or community score.

In exemplary embodiments, the system is configured to be operable for prescribing and/or applying artificial electromagnetic waves and/or fields to help support plant growth and health and/or to help support health, safety, and/or treatment of at least one entity within the community. The community may be an off-Earth community. And the system may be configured to be operable for prescribing and/or applying artificial electromagnetic waves and/or fields to help support plant growth and health and/or to help support health, safety, and/or treatment for the at least one entity within the off-Earth community to preempt and/or lower the risk of a future occurrence of a psychological episode, which may be linked to reduced protection of the Earth's electromagnetic field coupled with a solar event(s).

In exemplary embodiments, the plurality of different devices, sensors, other systems, and/or communications network(s) comprises at least one digital agent configured to be operable for developing, proffering, and/or implementing recommendations for altering behavior of at least one entity of the community which altered behavior will benefit the community in terms of land management, environment, and/or climate.

In exemplary embodiments, the system is configured to be operable for developing, proffering, and/or implementing recommendations regarding seeds including saving sprouts and recovery along with breeding and genetic modification non-earth gravity.

In exemplary embodiments, the system is configured to determine, through a plurality of measurements/readings taken by the plurality of different devices, sensors, other systems, and/or communication network(s) and/or through information from and/or about system inputs, behavior(s) of at least one entity of the community and: (a) context(s) associated with the behavior(s) of the at least one entity; or (b) location and the context(s) associated with the behavior(s) of the at least one entity. The at least one entity comprises one or more of a human, an animal, a plant, another system, a machine, a robot, an artificial intelligence, a virtual agent, a corporation, a business entity, a nation, a network, a driverless vehicle, a connected vehicle, a drone, and/or a governmental entity.

In exemplary embodiments, the system is configured to: assess, evaluate, and predict a risk of a future occurrence(s) of context(s) associated with behavior(s) by the at least one entity; and facilitate one or more actions and/or activities to preempt and/or lower the risk of a future occurrence(s) of context(s) associated with behavior(s) by the at least one entity before the context(s) associated with the behavior(s) occurs when the behavior(s) by the at least one entity is determined to be detrimental to the community.

In exemplary embodiments, the system is further configured to dynamically and adaptively determine a reward for incentivizing context(s) associated with behavior(s) of the at least one entity that is beneficial to the community; and/or the system is further configured to dynamically and adaptively determine a disincentive for disincentivizing context(s) associated with behavior(s) of the at least one entity that is detrimental to the community.

In exemplary embodiments, the system is configured to capture the behavior(s) and the context(s) of the least one entity in a system of record for tracking, managing, and redeeming reward(s) and disincentive(s). The behavior(s) of the at least one entity includes a contribution of the at least one entity to the community as it relates to the planting, growing, treatment, harvesting, producing, acquiring, generating, distribution, storage, and/or consumption of consumable resources of the community. The system is configured to quantitatively and qualitatively assess the contribution of the at least one entity to the community and assign contribution assessment, score, level, or metric(s) where the contribution of the at least one entity is measured against one or more participation and/or performance targets after the at least one entity's participation and/or performance has been adjusted to incorporate contextual situations, issues, and/or other factors resulting in a context outside expected parameters that may have impacted the at least one entity's participation and/or performance. And the system is configured to assign, award, and/or allocate an electronic or physical store of value for the contribution of the at least one entity to the community, which may be based in part on a comparison of the contribution assessment, score, level, or metric(s) assigned to the contribution made by the at least one entity with the contribution assessments, scores, levels, or metrics assigned to contributions made by other entities of the community.

In exemplary embodiments, the system of record may be a ledger, distributed ledger or blockchain system. The electronic or physical store of value may comprise one or more of money, a ticket, cryptocurrency, and a credit for a certain amount/type of consumable. The electronic or physical store of value may be transferrable from one entity to another entity. The electronic or physical store of value may be redeemable for cybercurrency and/or transferrable to an electronic wallet.

In exemplary embodiments, the at least one entity's participation and/or performance is measured against one or more participation and/or performance targets for a specialty farming and/or agricultural production process as a whole or for a combination of less than all of the steps, parts, and/or sub-processes of the specialty farming and/or agricultural production process.

In exemplary embodiments, the system is configured to capture to every contribution made by entities to the community relating to the planting, growing, treatment, harvesting, producing, acquiring, generating, distribution, storage, and/or consumption of consumable resources of the community. The system is configured to quantitatively and qualitatively value the contributions made by the entities to the community where the contributions are measured against one or more participation and/or performance targets once the participation and/or performance has been adjusted to incorporate contextual situations, issues, and/or other factors resulting in a context outside expected parameters that may have impacted the participation and/or performance. And the system is configured to assign, award, and/or allocate electronic or physical stores of value for the contributions made by the entities to the community, which may be based in part on a comparison of the contribution assessments, scores, levels, or metrics assigned to the contributions made by the entities of the community. The electronic or physical stores of value assigned, awarded, and/or allocated by the system for the contributions made by the entities to the community comprise credits for certain amounts/types of consumables, thereby allowing for merit-based allocation of the consumables to the entities of the community, whereby the credits may be tied to financial cost of the consumable, social impact, nutritional requirements individually or broadly, or other variables(s). For example, the credits may be for various nutrients, which may include tracking of various account balances and conversion back into a regenerative system or a financial accounting.

In exemplary embodiments, the system is configured to be operable for identifying a root cause(s) of a given negative measure and for qualifying, reducing an impact of, and/or otherwise nullifying a negative metric when the root cause(s) demonstrates that the root cause(s) was out of the control of the at least one entity; and/or the system is configured to quantitatively and qualitatively assess contributions made by entity(ies) to the community and assign contribution assessment, score, level, or metric(s) based on fixed objective criteria and criteria relative to other entity(ies) including other persons, robots, and/or assets of the community.

In exemplary embodiments, the system is configured to capture to contributions made by entities to the community relating to the planting, growing, treatment, harvesting, producing, acquiring, generating, distribution, storage, and/or consumption of consumable resources of the community. And the system is configured to quantitatively and qualitatively value or score the contributions made by the entities to the community for helping to determine allocation of the community's resources.

In exemplary embodiments, the plurality of different devices, sensors, other systems, and/or communications network(s) comprises at least one digital agent associated with at least one entity of the community. The system may be configured to search for, detect, measure, monitor, track, control, manage, react, and/or respond to one or more triggers of a human asset of the community by utilizing the at least one digital agent. The digital agent may be configured with specialized machine learning/AI-based capability(ies) to focus on searching for, detection, measuring, monitoring, tracking, controlling, managing, reacting, and/or responding to a particular trigger and its related triggers by utilizing data sets associated with the searching for, detection, measuring, monitoring, tracking, controlling, managing, reacting, and/or responding to the one or more triggers and their related triggers by utilizing and/or combining with data associated with the human's historical interaction of and/or association with said one or more triggers and their related triggers, including occurrences of the one or more triggers and their related triggers, the behaviors and contexts preceding, causing, associated with, or resulting from the occurrence of the one or more triggers and their related triggers. The contexts may include one or more of where, when, why, who, what, and/or how associated with the behavior(s) preceding, causing, being associated with, and/or resulting from the occurrence of the one or more triggers and their related triggers. And the one or more triggers and their related triggers may be associated with mental and/or physical behavior and/or situational ranges, levels, break points, targets, or milestones that, once reached or increasing in risk of being reached and/or occurring, can materially increase the potential for changes in behavior that can cause negative or positive results associated with that change in behavior.

In exemplary embodiments, the digital agent may be local to the at least one entity including embedded, attached, remote, and/or cloud based that communicates with surroundings using a variety of sensors and/or interfaces. The digital agent may comprise one or more specialty trigger agents configured to interact with one or more other specialty functional agents, such as for location and context. The digital agent may comprise one or more specialized agents configured to focus on location and context detection, historical tracking, future location and/or context prediction and action generation. The digital agent may be configured to predict future location based on historical and current location and associated contexts for both the at least one entity individually as well as other entities that have in the past encountered similar location/contexts. The digital agent may comprise an artificial intelligent (AI) assistant-type bot, assistant, aid, or agent. The digital agent may comprise an intelligent agent (IA) configured to perceive its environment, to take action(s) autonomously in order to achieve a goal(s), and to improve its performance with learning and/or knowledge. The digital agent may be configured to perceive its environment through the plurality of different devices, sensors, other systems, and/or communications network(s) and to act upon that environment through one or more actuators.

In exemplary embodiments, wherein the digital agent may comprise one or more of a specialty agent or bot dedicated to one or more particular functions and/or data specialty areas, a Medical Bot specialized within a medical field(s), a cancer diagnostic bot, a sepsis treatment bot, a location analysis and prediction bot (Chat GPS), a forensics bot, a context determination bot, a trigger detection bot, a behavior determination bot, a mental state bot, and/or a digital agent comprising a natural language processing tool driven by AI technology. In exemplary embodiments, the digital agent may comprise a bot configured to focus on triggers, trigger detection, and alternative actions based on trigger and context.

In exemplary embodiments, the system is configured to: determine, through a plurality of measurements/readings taken by the plurality of different devices, sensors, other systems, and/or communication network(s) and/or through information from and/or about system inputs, contexts, and current available and utilized resources of a community, and current and future needs of a community, possible additions, changes, maintenance, and/or removals of one or more resources to and/or from the community; and cross reference and assess the one or more current resources of the community with the current and future needs of the community to determine whether the community should expand, maintain, and/or reduce one or more resource production. The system is configured to direct, advise, or otherwise instruct the community to expand, maintain, and/or reduce/remove productive capabilities when the system determines that the community should expand, maintain, and/or reduce a resource(s) production required to support said future needs of the resource(s).

In exemplary embodiments, the production utilizes the conversion and/or transformation of regolith into a usable soil state(s) utilizing a plurality of physical weathering, chemical weathering, biological weathering, worm deployment, microbe application, biological process, water/hydration application, pioneer species utilization, algae growth, and/or fungi-based, synthetic, and/or gene engineered organism introduction(s), action(s), method(s), technique(s), mechanism(s), and/or mode(s) of processing. The conversion, transformation, and/or utilization introduction(s), action(s), method(s), technique(s), mechanism(s), and/or modes of processing are conducted in one or more contexts enabling acceleration in time and/or quantity and/or scale of such conversion and/or transformation of the regolith and/or utilization of the resulting soil(s). One or more production contexts include one or more natural, artificial, and/or constructed structures, systems, machinery, mechanisms, and/or environments with systemic ability to control one or more aspects of moisture, water interaction, humidity, radiation, light, nitrogen, oxygen, CO2, mineral content, heavy metals, PH, nutrient content, particle size, particle distribution, particle composition, organic matter, microbiome, fungi, aggregation/structure, cation exchange and capacity. The needs include physical, mental, nutritional, hydration-related needs used in and for human consumption, at various levels including necessary for survival for one or more humans, maintenance of status quo, ability to support various population levels and demographics, individual survival and/or physical and mental well-being. Resources include raw materials, regolith, minerals, nutritional-related, light-related, water-based, fertilizers, worms, chemical agents, pharmaceutical agents, microbes, pioneer species, algae, fungis, synthetic organisms, gen-engineered organisms, labor, capital, space, capacity, processing, storage, and transportation capabilities, and interim and end products and/or growth and/or production resulting from use of one or more resources.

In exemplary embodiments, the community is an off-Earth community. The system is configured to be operable for managing agriculture of the off-Earth community. When the system determines that the off-Earth community should expand resource production, the system is configured to direct, advise, or otherwise instruct the community regarding: In-Situ Resource Utilization (ISRU); transformation of local resources into supporting materials for production of bioproducts and support of community health and longevity; transformation of local resources using ISRU methods including transformation of off-Earth regolith material into locally derived soils to support IRSU based agriculture for the community; and/or transformation of regolith material including one or more biological(s), chemical alteration, physical alteration, another method of alteration, and combinations thereof.

In exemplary embodiments, the system is configured to: monitor, through a plurality of measurements/readings taken by the plurality of different devices, sensors, other systems, and/or communication network(s) and/or through information from and/or about system inputs, health of worms that are used to facilitate transformation of off-Earth regolith material into locally derived soils to support IRSU based agriculture for the community; and determine and facilitate one or more actions, if any, for improving health of the worms.

In exemplary embodiments, the system is configured to provide guidance or direct expansion of productive capacity using data points including context provided by one or more of an IoT device(s), sensor(s), health data, predictive modeling, biomarker(s) such as for pH or water retention, soil cation exchange, particle size or distribution, or other method.

In exemplary embodiments, during expansion of productive capabilities of ISRU soils, the system is configured to account for the expanding capacity in a larger model to support the community and anticipate and suggest best use(s) of the new expanded capacity including one or more of growing staple crops, expanding to specialty goods, more advanced bioproducts (such as supporting biopolymer creation) and/or creating a greenspace for psychological health and well-being.

In exemplary embodiments, the context includes a physical and/or virtual geofence or boundary area, a contextual geofence or boundary area, and/or restrictions associated with that geofence(s) or boundary area(s), and/or restriction(s) associated with the geofence(s) or boundary area(s). The geofence or boundary area may include a buffer zone with a varying physical or virtual length, width, and/or height.

In exemplary embodiments, the community comprises a neighborhood that is crime-ridden and/or subject to a turf war between rival gangs. The system is configured to: dynamically and adaptively determine a reward for incentivizing behavior for an associated context that increases a likelihood of a future occurrence(s) of a respectful behavior(s) by the at least one entity that is respectful of at least one other entity of the community before the respectful behavior(s) occurs; and/or dynamically and adaptively determine a disincentive for disincentivizing behavior for an associated context that decreases the likelihood of a future occurrence(s) of a disrespectful behavior(s) by the at least one entity that is disrespectful of at least one other entity of the community before the disrespectful behavior(s) occurs.

In exemplary embodiments, the system is configured to dynamically and adaptively determine a reward for incentivizing behavior for an associated context that increases a likelihood of a future occurrence(s) of a respectful behavior(s) by the at least one entity that is respectful of at least one other entity of the community before the respectful behavior(s) occurs and facilitate redemption of the reward including one or more of a material reward, a physical reward, a financial reward, a monetary reward, an electronic reward, a virtual reward, a non-material reward, and a non-financial reward. Additionally, or alternatively, the system is configured to dynamically and adaptively determine a disincentive for disincentivizing behavior for an associated context that decreases the likelihood of a future occurrence(s) of a disrespectful behavior(s) by the at least one entity that is disrespectful of at least one other entity of the community before the disrespectful behavior(s) occurs and facilitate redemption of the disincentive including one or more of a material punishment or penalty, a physical punishment or penalty, a financial punishment or penalty, a monetary punishment or penalty, an electronic punishment or penalty, a virtual punishment or penalty, a non-material punishment or penalty, and a non-financial punishment or penalty.

In exemplary embodiments, consumables of the community may include one or more of food, liquids, minerals, and environmental needs of living organisms in the community needed to sustain existence and health. The system may be configured such that the consumables are organized virtually and/or physically into modules which are based on one or more of: physical attributes, sustenance impact, ability-to-produce conditions and contexts, living entity(ies) consuming the consumable, geographical location, raw material inputs and/or partial or finished good output storage requirements, tools and materials needed for production, growing life cycle, growing temporal requirements, shelf-life duration, expiration expectations, and/or hybrids and combinations thereof.

In exemplary embodiments, the system may be configured such that consumables of the community are organized virtually and/or physically into modules which include one or more units of seed(s), plant(s), animal(s), mineral(s), liquid(s), and/or gas(es) that can directly or when combined with other produce and/or result in one or more unit(s) of consumable items. The system may be configured to be operable for managing the community by utilizing needs-based production and consumption capabilities that include: consumables needed to sustain the existence and health of an individual living entity, group(s) or pools of individual living entities, and/or the community of living entities as a whole based on their nutritional needs, living/working environments and contexts, physical and/or mental well-being, ability to interact with other members of the community, and/or ability to contribute to the community in balance with resources needed for support; and/or condition(s) with, within, or associated with one or more living entity(s), module(s) of production and/or consumption, or related input, in-process, or output of production and/or consumption experience, including situations, circumstances, environments, and states of persons, places, and things.

In exemplary embodiments, the system is configured to be operable for: monitoring, via the plurality of different devices, sensors, other systems, and/or communications network(s), every entity's interaction with every other entity of the community to determine the interaction impact on one or more community triggers including one or more of anxiety, depression, conflict, and/or motivation level; and weighing or scoring the monitored interactions, which may include likelihood of a future occurrence, associated criticality for the community, and whether an identified root cause(s) of a given negative measure demonstrates that the root cause(s) was outside or at least partially outside of the entity's control.

In exemplary embodiments, the system may be configured to be operable for detecting trigger(s), diagnosing root cause(s) of the trigger(s), and developing resource-optimized remedies/actions at the multi-entry level for the community. The system may be configured to influence connected actions between entities of the community such that the outcome of said actions facilitates the achievement of a common goal, objective, motivation, or purpose for the community. The system may be configured to facilitate, control, and/or manage interaction between entities of the community by utilizing a plurality of measurements/readings taken by the plurality of different devices, sensors, other systems, and/or communications network(s). The system may be configured to dynamically redeploy measurement capabilities of the plurality of different devices, sensors, other systems, and/or communications network(s) automatically without manual human intervention or with little human manual intervention.

In exemplary embodiments, the system is configured to calibrate measurement capabilities and results of the plurality of different devices, sensors, other systems, and/or communications network(s) automatically without manual human intervention or with little human manual intervention to accommodate for different context(s) of the community under which the plurality of different devices, sensors, other systems, and/or communications network(s) are being used. The calibration provides the ability to compare data obtained for the community via the plurality of different devices, sensors, other systems, and/or communications network(s) with data obtained elsewhere under different contexts.

In exemplary embodiments, the system is configured to implement a digital twin(s) for an individual(s) of the community that seeks to minor behavior and associated contributions of the individual to the community.

In exemplary embodiments, the system is configured with and/or includes a project and work management system operable for harmonizing or providing harmony between human and robotic entities of the community. The project and work management system includes one or more digital agents associated with and/or working on behalf of one or more humans of the community, and one or more digital agents associated with and/or working in coordination with one or more robots of the community. The digital agents are operable for coordinating human/robot behaviors for given tasks in given contexts.

In exemplary embodiments, the system is configured for administering and managing a community's prison-less criminal, civil, and other legal/judicial sentence(s) and/or verdicts and associated punishments associated with an infraction(s) and a violator(s) utilizing community(s), victim(s), and violator(s) needs-based, context-based, and behavior-driven contribution and feedback capabilities and associated punishment and community contribution scoring management capabilities. The plurality of different devices, sensors, other systems, and/or communications network(s) are configured to dynamically and flexibly manage the needs, behaviors, contexts, triggers, and resource utilization/usage associated with the infractions and/or associated punishment to satisfy, reduce, and/or modify said punishments and/or pre-empt, prevent, and/or mitigate current and/or future needs, behaviors, triggers, and/or resource utilization/usage, and associated contexts, and pre-empt, identify, proffer, recommend, implement, and/or facilitate additions, modifications, and/or deletions to a violator's needs, behaviors, triggers, and/or resource utilization/usage and associated contexts that will satisfy, reduce, and/or modify said punishments, and/or benefit the community, the victim(s) of the infraction(s), and/or the violator(s) themselves.

In exemplary embodiments, the system is configured to be operable with digital agent monitoring for key variables, calculating future requirements (e.g., resource, amounts, dosages, timing, automatically administering dosage through modifying medicine administration, menu selection, meal preparation, etc.), submitting those requirements to the overall production system, monitoring its production, monitoring its production and reallocation to the user(s). In effect, the digital agent is performing various functions with respect to a particular person, issue, ailment, etc. and interfacing with the overall system to ensure (as best as possible) that the needs of the user(s) are met as close to the user(s) requirements as possible. In exemplary embodiments, the system may include a module comprising one or more digital agents focused on managing the particular consumption needs(s) for a user(s) including detecting/determining/calculating one or more needs for the user(s) in the form of resource requirements, interfacing with the rest of the system to input (and possibly advocate) for the user(s) for those requirements. The digital agent(s) may potentially join or unite with other digital agents to pool their requirements, in effect becoming a requirements advocacy block to obtain their collective requirements for a particular resource as a reallocation into the overall food supply chain. The digital agents may monitor how the system incorporates the requirements if or in what form, monitor the production related to that module as well as any attempts to divert from it, ensure its production with respect to its user(s) requirements, and upon production making sure that the user(s) obtain the produced resource in the amount and from that it needs, as well as controlling the administering of the resource, such as direct injectables, additives to food, changing of menu to incorporate resource, etc.

For example, a person's digital agent could have a variety of modules in it, (e.g., submodules) or separate user modules within more general Anxiety modules our modeled the trigger concept disclosed herein, e.g., a digital agent module (for each community member, as well as one tracking and issue/trigger overall for the community, or pools of that trigger's sufferers, etc.) for all things Anxiety, Anger, Boredom, etc. The digital agent module(s) would focus on monitoring (and refining the monitoring) of the user, detecting a rising level of anxiety of its user for example by detecting more aggressive, terseness in the user's language, various body measurements, even monitoring conversations or messages of co-workers that are commenting on the demeanor of the user.

The digital agent/modules may be configured to develop an anxiety-reducing plan for the user by also analyzing past behavior of the user and/or similar people (for example, if the user is a maintenance engineer in a somewhat dangerous job, assessing other people with a similar job and contexts). This could include, for example, including interfacing with medical agents or other medical resources, that the agent forecasts that the user will need to add 10 mg of Passion Flower to the user's diet within 3 months. It would interface with the rest of the system to input those requirements into the resource allocation and production schedule for medicinal herbs or other logical control aspect related to the production of passion flower.

The agent interface could include “negotiating” with the system to get the needed allocation. This could take many forms, including bartering or using other resources currently allocated to the user to “pay for” this reallocation. It could also interface with other agents that also are detecting anxiety needs for their users to essentially pool their requirements (as production of some resources may greatly benefit from scaling their production. Some production may not be possible at all unless it reached some minimum amount. The “negotiation” might include determining acceptable substitutes in cases where the full desired allocation of the resource is not possible; for example substituting some amount of lavender in the place of the portion of the passion flower requirement. Indeed, the ability of these digital agents to interact with each other, while potentially using any architecture and system of integration, would likely be in the some form of distributed mesh architecture with every specialized agent being able to interact with any/every other agent on an as-needed basis as determined by the agent. The as-needed basis would be particularly triggered by the occurrences of certain triggers, the behaviors underlying/causing/contributing to those triggers, and the context in which they occurred, whether occurring in everyday activities or caused by certain unusual events, e.g., unplanned internal or external activities, disruptions, etc. that may have a measurable impact on the community or parts of it, ranging from full scale disasters, to breakdown in key equipment, to new people entering the community or other people exiting (or dying). The ability to not just absorb but understand the implications as well as how to respond to all of this information is beyond most human-run systems, but would be within the capabilities of machine learning/AI, such as in the form of digital agents.

These agents/modules would interact with other digital agents, such as ones responsible for “overseeing” the Anxiety levels of the community or parts of the community, as well as digital agents focused on all aspects of certain food/production segments, like medicinal herbs, or specific herbs, and/or food that requires certain resources/resource amounts, and/or particular resources themselves. For example, each user may have an agent/module that are distinguishable for a user, each with his/her triggers (e.g., anxiety), behaviors (e.g., what demonstrates anxiety), context (e.g., what contexts trigger anxiety), needs (e.g., what is needed to preempt/mitigate the anxiety). More broadly, the community in itself may be provided with “specialty agents” that focus on particular triggers, behaviors, contexts, and needs, and what resources are needed to meet the needs. The community's network of digital agents may be configured to focus on a particular need, context, behavior.

In exemplary embodiments, the needs include needs of an entity comprising physiological needs, safety needs, love and belongingness needs, esteem needs, self-actualization needs, and self-transcendence needs. Physiological needs refers to needs that are most basic and critical needs for survival including food, water, air, sleep, shelter, and sex. Once physiological needs are met, safety needs refers to people seeking security and stability including physical safety (protection from physical harm or danger), financial security (having enough resources to cover basic needs), health and well-being (access to healthcare and a sense of well-being), and stability (predictability in life and a safe environment). Love and belongingness needs revolve around social connections and relationships and include friendship (building and maintaining meaningful friendships), intimacy (forming close, romantic relationships), family (feeling a sense of belonging within a family unit), and community (being part of social groups, communities, or teams). Esteem needs involve self-respect and recognition from others and include self-esteem (developing a positive self-image and self-worth), recognition (receiving acknowledgment and respect from others), achievement (striving for accomplishments and success), and status (seeking social or professional status and recognition). Self-actualization needs are about personal growth and fulfillment and include creativity (expressing one's creativity and unique talents), problem solving (engaging in challenging tasks and problem-solving), morality (exploring personal values and ethics), and acceptance of facts (embracing the realities of life). Self-transcendence needs include altruism (a focus on helping and benefiting others), spirituality (seeking a deeper connection with the spiritual or transcendent), and service (engaging in activities that contribute to a greater good).

Other key aspects of specialized communities that may be addressed by the systems disclosed herein include rewarding/disincentivizing behaviors; preempting criminal, addictive, and generally undesired behavior; and social management. In specialized communities, there are very significant risks of not only full-scale disasters (such as a radiation event that endangers a large crop), but smaller scale disasters or even various form of unusual “events” that have the potential to disrupt the community. Particularly since many such communities tend to be relatively small, any individual/small numbers of people “event” (physical or mental) has the potential of rippling through the community in significant ways, particularly if its resolution requires (re)allocating/directing scarce resources to address it. Some sort of scoring system may be implemented to objectively (to the extent possible) address the event in a way perceived as fair to the larger community. In the same vein, criminal activity will have an outsized impact on the community, as there will be limited resources to imprison criminals, and indeed a handling a criminal may take up more resources than the average community member exasperated by the fact that the criminal.

Further, it is likely that some of the social disfunction, if not criminality, in a small community will be among small groups of individuals. Further, like much of the social upheaval and ultimately crime that occurs today, its root cause will be in seemingly petty disputes among individuals, ones that are perceived as “disrespectful” and require some sort of response (e.g., revenge), that escalates until there is serious injury or even death among the parties. This kind of perceived social disrespect and the escalation of response, including drawing other individuals into it (akin to gangs or at least cliques), if allowed to occur, will function as a cancer on the small community, as it consumes resources that would be far better utilized in other ways.

One way of addressing all of the above would be to have a kind of community contribution scoring system to measure each individual's contribution to the community, as well as score (deduct) behaviors that are detrimental to the community. While at a high level, such a system would seem to make each community member in competition with all the others in a bit to “outrank” others and thus score privileges that lower scoring members would not attain, that would not be the intent of the system. For example, such a system may not even be implemented until someone commits an infraction, particularly against another. In such instances, a “mini-community” might be established that involves everyone associated with the infraction, to measure their behaviors and award (or deduct) points depending on adherence (or not) to certain rules and conditions. It could also enable ways of “evening the score” between supposedly disrespected parties involved in a dispute, even monetarily, such as deducting community credits (perhaps using blockchain and crypto currencies in some form) to compensate the “wronged” party before they have the time, inclination, and opportunity for revenge.

Such as scoring system for use in social management could also be used in the digital agent “bartering” scheme discussed herein, where a user (e.g., community member) who is anticipated to need more than their allocated form of resources could effectively buy or barter say “anxiety credits” that could “fund” the acquisition of an extra passion flower allocation for example. Disclosed herein are exemplary embodiments of comprehensive and fair systems of measuring behavior, contribution to social stability, and community contribution as a whole.

The ability to appropriately punish criminals/criminality of various forms and severity will be a particular difficulty in closed communities. Expelling them from the community would in all practicality not be an option for many of the communities (e.g., a space station, moon base, other off-Earth location, etc.), but other traditional punishments, notably prisons would be impractical for all but the most heinous crimes, particularly with respect to incarceration and taking a resource out of the community contribution chain but keeping them in the food and other resources chain. Indeed, studies on Earth for “normal” prisons have a criminal consuming at minimum of $50K worth of resources per year. In a specialty community, a prisoner would need all sorts of resources: not just property, facilities, food, water, medical and labor to guard them, but also oxygen and specialized sanitation. Indeed, since much of the above (a portfolio of resources dedicated to one individual) would go against the practical reality in managing a specialty community.

In an emergency/extreme event, where the resources of the community suddenly become even more limited, there will likely be a great temptation on part of at least part of the society (particularly those likely to panic or become mob-like in their mental outlook) to drastically, even totally cut back on the resources allocated to an incarcerated criminal, given their negative community contribution. In exemplary embodiments, various digital agents may be configured to anticipate this, and, depending on the nature of the emergency/event, the resources impacted by the emergency/event, and the degree to which the criminal consumes directly or indirectly those resources, significantly reduce the incarcerated criminal's resources, at least to a minimum sustenance level. This cutback would need to be made public to the community to preempt negative actions on their part. Additionally, or alternatively, the digital agents (e.g., the digital agents for the criminal, the digital agents managing the community resources, etc.) may be configured to be operable for tracking and allowing the criminal to gain goodwill credits that may help him/her negotiate time off their sentences. Accordingly, alternatives will be needed (even more so than in a “normal” community for the criminal (perhaps varying on the state of the criminal's sentence, or even if they have been convicted yet-see separate comment on criminal forensics) to engage in other methods of serving their sentence short of incarceration. Such activities/behaviors (instead of incarceration) could include (but are not limited to):
Community Service: Offenders can be required to perform community service as a way to give back to the community. This service would likely need to focus on the most urgent needs of the community (particularly if in emergency/extreme event)). This could involve cleaning up debris, cleaning public spaces, helping the needy/elderly, or participating in environmental projects.
Fines and Restitution: Offenders may be fined, and the funds collected can go towards compensating victims or supporting community programs and services. This may be tied to the “respect” part where the restitution is at least in part geared towards preventing revenge/retribution (the revenge/retribution activities do not have had to be criminal in nature to qualify for such retribution). Such fines and retributions could be “earned”, e.g., retribution activities could be tracked via blockchain, etc.
Probation: Offenders can be placed on probation, requiring them to meet certain conditions, such as regular check-ins with a probation officer, attending rehabilitation programs, or maintaining a clean record.
Restorative Justice Programs: Encourage dialogue between offenders and victims to understand the impact of the crime. This can lead to restitution agreements and a focus on repairing the harm done.
Education and Skill-Building Programs: Provide offenders with opportunities to acquire new skills, education, or job training to reduce the likelihood of re-offending.
Electronic Monitoring: Use technology such as ankle bracelets to monitor the whereabouts of offenders, particularly for non-violent offenses.
Counseling and Rehabilitation: Offer counseling and rehabilitation programs for individuals involved in minor crimes, addressing underlying issues like substance abuse or mental health problems.
Community-Based Corrections: Establish halfway houses or supervised living arrangements where non-violent offenders can receive support and guidance while reintegrating into the community.
Community Panels: Set up community panels or boards composed of local citizens to decide on appropriate sanctions for minor offenses, ensuring community involvement in the process.
Mediation and Conflict Resolution: Utilize mediation and conflict resolution techniques to address disputes and conflicts without resorting to punitive measures.

In exemplary embodiments, the system is configured to include community scoring (e.g., social credit scoring, etc.) in which scores may be calculated or determined depending on at least one or more of the following.
The ability to (via sensors, networks, AI, etc.) monitor, measure, and track anything and everything that a person does that could contribute to (or deduct from) the person's contribution to society. This might include having various tiers or hierarchies of tracking (which consumes resources by themselves), particularly (but not necessarily for) detections. One exemplary way of punishing a person might not necessarily involve any of the above fines/restation activities (or it might reduce them), but instead involve the implementation of such “detection-tracking” at various levels of severity/intrusiveness. A misdemeanor might involve tracking locations and times, e.g., deductions and associated monitoring associated with monitoring teenage social hours and locations for example, and what the person does during those times/locations, like drinking, particularly as they related to the original infraction, such as vandalism the premise being the person's infraction of vandalism occurred while drinking.
A “ledger” (e.g., blockchain, etc.) that is incapable of hacking/political manipulation/human error and exaggeration would be included in exemplary embodiments. Sensors, networks, etc. measurements, as well as negotiations and decisions made (by agents and others) would all be captured in effect a running log of everything a person (or thing) does in the community on a daily basis, perhaps at various tiers of tracking. This will allow an impartial record of the raw data of a person's contribution (or deduction) to/from the community to be on record, and to use for various purposes not only (semi or actual) real-time but after the fact as needed (such detection of and setting of/administering of retribution).
The scoring system is configured to allocate resources fairly and in the process have the public know that resources are being allocated fairly, there may be a publicity/public knowledge aspect to the scoring system as well as the behaviors/activities underlying it. It also will allow “rescoring” or similar to recalculate a community score upon changing of a scoring system(s) inputs, algorithms, and outputs. In exemplary embodiments, there may be a digital agent/system by itself dedicated to continually monitoring and tweaking the scoring system as more information about the community's situation is collected and/or the community's resources situation changes. One example would be publicizing the person's score and/or infraction, at least to a certain group of people. While such “public shaming” may be controversial, it has shown to be an effective tool, and requires little resources in itself as a tool.
Related to fines/restitution would be in associating a value with a community scoring “point” or particular activity/behavior/contribution by the person (perhaps using blockchain as the system of record) through the use of crypto. One example way of doing this is that a portion of the person's redeeming activity (e.g., use human related power to charge battery(ies), etc.) is allocated to powering a certain amount of time and/or effort on part of a crypto miner system. This could perhaps also involve reallocated energy resources that go into powering the criminal's housing allocation, for example. In this way, there is a way to financially “value” a person's contribution and be able to redirect/repay or otherwise use the “fruits of that labor” in a broader financial ecosystem. Accordingly, exemplary embodiments disclosed herein include a system that is configured to be operable for selecting, collecting, monitoring, and contextualizing a person's behaviors using blockchain in a scoring system to directly or indirectly fund/power the generation of an amount of cryptocurrency.
Because it may be very difficult to measure one person's contribution relative to another person's and/or a metric, exemplary embodiments may include a mechanism for normalizing the activity/behavior/contribution. This could involve normalizing the activity against absolute or relative measures of the activity/behavior/contribution that others make, with “others” being further narrowed to reflect demographics, geography, timeframes, and in general contexts in which the activity/behavior/contribution is made. Further, the contexts themselves may need to be normalized. For example, an hour's worth of digging could be measured by amount of soil displaced, and could be further normalized by the context, e.g., 30 minutes of digging on the surface of the moon in a spacesuit is far different than digging in a nice-controlled atmosphere. A 100-pound woman would not expect to dig as much a 200-pound man; a 200-pound man with a back ailment would not be able to dig as much/as fast as a 200-pound man in good health, etc.

Sanitation/sewage in a closed specialty community is an important aspect that may be addressed by exemplary systems disclosed herein. A person's “output” may need to be monitored/measured, for a variety of purposes including early detection of an illness, but also to what degree(s) the output is true “waste”, e.g., what has to be discarded and thus a drain on the community resources versus what can be recycled (e.g., water, which depending on the composition of the output could be of varying difficulty to recycle) versus what may actually contribute to the community (e.g., fertilizer). In exemplary embodiments, an “output score” would be determined or calculated individually as well as a composite score as part of an overall community contribution score. This score could be used in a multitude of ways, from everyday resource allocation (e.g., where you get to live in the community and the resources associated with it) to “normal” resource reallocation (such as the passion flower example above), to emergency/extreme event allocation, to criminal/retribution, etc.

In exemplary embodiments, a system is configured for administering and managing a community's prison-less criminal, civil, and other legal/judicial sentence(s) and/or verdicts and associated punishments associated with an infraction(s) (e.g., crime, etc.) and a violator(s) (e.g., criminal, offender, transgressor, etc.) utilizing community(s), victim(s), and violator(s) needs-based, context-based, and behavior-driven contribution and feedback capabilities and associated punishment and community contribution scoring management capabilities. The system comprises a plurality of different devices, sensors, other systems, and/or communications network(s) configured to dynamically and flexibly manage the needs, behaviors, contexts, triggers, and resource utilization/usage associated with the infractions and/or associated punishment to satisfy, reduce, and/or modify said punishments and/or pre-empt, prevent, and/or mitigate current and/or future needs, behaviors, triggers, and/or resource utilization/usage, and associated contexts, and pre-empt, identify, proffer, recommend, implement, and/or facilitate additions, modifications, and/or deletions to a violator's needs, behaviors, triggers, and/or resource utilization/usage and associated contexts that will satisfy, reduce, and/or modify said punishments, and/or benefit the community, the victim(s) of the infraction(s), and/or the violator(s) themselves.

In exemplary embodiments, the system is configured for utilizing a plurality of digital agents that measure, monitor, track, assess and/or analyze a violator(s), victim(s), and/or community(s) needs, contexts, behaviors, triggers, and/or resource utilization/usage to develop, proffer, facilitate, and/or implement contribution, feedback, and/or punishment reduction recommendations associated with the needs, contexts, behaviors, triggers, and/or resources using a scoring capability(s).

In exemplary embodiments, the system is configured for using the plurality of digital agents in the management of said prison-less system including identifying, recommending, facilitating, and/or otherwise implementing action(s) for the violator(s) to take that will increase the likelihood of a positive impact to the community, victim(s) and/or violator(s) and/or that will decrease the likelihood of a negative impact to the community, victim(s), and/or violator(s).

In exemplary embodiments, the system is configured for using a ledger-based transaction capture system including a ledger, distributed ledger or blockchain system for recording measurements, determinations, estimates, monitoring, and/or tracking of the violator(s), community(s), and/or victim(s) needs, behaviors, triggers, contexts, resource utilization/usage, punishments, recommendations, recommendations adherence and associated values, inputs to a score, and scores/scoring.

In exemplary embodiments, the system is configured such that the contribution and feedback capability(s) and/or associated punishments include one or more of community service, fines and restitution, probation, restorative justice, education and skill-building, electronic monitoring, counseling and rehabilitation, community-based corrections, community panels, and/or mediation and conflict resolution capabilities.

In exemplary embodiments, the system is configured such that the recommendations are associated with a value(s), score(s), and/or input(s) to a score based on its impact on and/or contribution to the community(s), victim(s), and/or violator(s) needs, behavior(s), triggers(s), context(s), and/or resource utilization/usage.

In exemplary embodiments, the system is configured such that the contribution and feedback capability(s), associated punishments, and/or community(s), victim(s), and/or violator(s) need(s), behavior(s), trigger(s), contexts and/or resources utilized/used are associated with a scoring system.

In exemplary embodiments, the system is configured such that the contribution and feedback capability(s), associated punishments, and/or community(s), victim(s), and/or violator(s) need(s), behavior(s), trigger(s), contexts and/or resources utilized/used are associated with one or more value(s), score(s), and/or input(s) to a score. Current or future violator(s), victim(s), and/or community contribution value(s), score(s), and/or input(s) to a score can be estimated (e.g., projected, determined, predicted, forecasted, etc.) based on the violator′ (s) past, current and/or predicted needs, behaviors, triggers, and/or resource utilization/usage, and associated contexts, and used to prioritize, weight, and/or otherwise influence the recommendations, and/or impact the violator(s) punishment(s) by decreasing, lowering, diminishing, reducing, and/or otherwise satisfying the punishment(s), and/or increasing, enhancing, adding to, and/or otherwise increasing in severity the punishment(s). A violator's punishment(s) and/or associated value(s), score(s), and/or input(s) to a score can be decreased, lowered, diminished, reduced, and/or otherwise satisfied, and/or increased, enhanced, added to, and/or otherwise increased in severity, depending upon the violator's need(s), behavior(s), trigger(s), and/or resource utilization/usage, and associated contexts, and/or to the degree, timeliness, effectiveness, efficiency, accuracy, and/or level of quality the violator follows, implements, and/or satisfies the system(s) recommendations. The system may be configured to allow sentences to be dynamically and flexibly reduced or increased based on what the violator does versus a traditional rigid system in which sentences are fixed as being either time based or monetarily based along with some sort of location safeguard (e.g., ankle bracelet, etc.). For example, the system may reward a violator with “extra credit” if the violator exceeds the recommendations and vice versa.

In exemplary embodiments, the system is configured for use with a plurality of violators such that: a behavior of one violator that has a negative impact to the community, victim(s), and/or violators is detrimental to the other violators; and a behavior of one violator that has a positive impact to the community, victim(s), and/or violators is beneficial to the other violators. Accordingly, the system may be applied to multiple violators such that any one violator's “good or bad” deeds affect all the multiple violators, thus providing a “shame/fear/peer pressure” factor to keep violators in line based on the knowledge that they can help or hurt the other violators depending on their behavior.

In exemplary embodiments, the system is configured such that: punishments, associated monitoring, recommendation development, recommendation adherence, and/or scoring is done in a tiered and/or hierarchal structure; and/or monitoring and/or estimating/predicting of a violator(s), victim(s), and/or community(s) needs, behaviors, triggers, and/or resource utilization/usage and/or associated contexts and application to a violator's punishment and/or victim(s) and/or community(s) contribution score(s) are done in a tiered and/or hierarchical structure.

In exemplary embodiments, the tiered and/or hierarchical structure is qualitative, quantitative, or a combination thereof. The tiered and/or hierarchical structure includes a scoring system(s) that may utilize one or more of a quantitative, numerical qualitative, color coding, binary, ordinal ranking, textual feedback, graphical representation(s), heatmap(s), percentage-based, checklist, threshold-based, range-based, risk matrices, machine learning/AI, natural language processing, sensor alarms, health-based and/or behavioral analysis algorithm.

The tiered and/or hierarchical structure may be configured to be tailored to needs, behaviors, contexts, location, triggers, and/or resource utilization. The system may be configured such that a recommendation and/or degree of adherence to a recommendation is weighted according to importance, criticality, and/or impact to the violator(s), victim(s), and/or community(s), whereby the weighting depends on the need, behavior, trigger, and/or resource utilization/usage and associated context. For example, the system may assign a value for picking up trash when convenient to the violator that is less than a value that would be assigned for picking up trash when inconvenient or difficult for the violator.

In exemplary embodiments, the system is configured to include a learning and feedback mechanism that enables the system to analyze and modify what is tracked/measured/scored, how the tracking/measurements/scoring is performed, how the tracking/measurements/scoring is performed, how recommendations are developed, and how a punishment/punishment score can be reduced/increased based on the system learning about the violator(s) and/or learning about changes in the needs of the victim(s), the community(s), and/or violator(s).

In exemplary embodiments, wherein the system is configured such that: the system is configured such that the contribution and feedback capability(s), associated punishments, and/or community(s), victim(s), and/or violator(s) need(s), behavior(s), trigger(s), contexts and/or resources utilized/used are associated with a scoring system; the system is configured to assign, award, and/or allocate an electronic or physical store of value based on a score(s) of the scoring system; and the electronic or physical store of value comprises one or more of money, a ticket, cryptocurrency, and a credit for a certain amount/type of consumable; and/or the electronic or physical store of value is transferrable from one entity to another entity; and/or the electronic or physical store of value is redeemable for cybercurrency and/or transferrable to an electronic wallet.

In exemplary embodiments, the system is configured with the ability for setting punishment(s) based on possible contribution(s) the violator(s) is able to make including contribution(s) made by the violator(s) that have a positive impact on need(s) of the victim(s) and/or community(s) thereby allowing the violator(s) to interactively reduce the punishment(s) and achieve rehabilitation by making the contribution(s) that have a positive impact on need(s) of the victim(s).

In exemplary embodiments, the system is configured such that a violator(s) one of more needs, behaviors, triggers, contexts, and/or resource utilization/usage can be measured, determined, estimated, monitored and/or tracked with such capability(s) being able to determine, estimate, and/or generate a community contribution score or a value associated with a community contribution score(s) for that need(s), behavior(s), trigger(s), context(s) and/or resource usage(s).

Exemplary embodiments may include one or more computing devices, such as one or more servers, workstations, personal computers, laptops, tablets, smartphones, person digital assistants (PDAs), etc. In addition, the computing device may include a single computing device, or it may include multiple computing devices located in close proximity or distributed over a geographic region, so long as the computing devices are specifically configured to function as described herein. Further, different components and/or arrangements of components than illustrated herein may be used in the computing device and/or in other computing device embodiments.

Exemplary embodiments may include one or more processors and memory coupled to (and in communication with) the one or more processors. A processor may include one or more processing units (e.g., in a multi-core configuration, etc.) such as, and without limitation, a central processing unit (CPU), a microcontroller, a reduced instruction set computer (RISC) processor, an application specific integrated circuit (ASIC), a programmable logic device (PLD), a gate array, and/or any other circuit or processor capable of the functions described herein.

In exemplary embodiments, a memory may be one or more devices that permit data, instructions, etc., to be stored therein and retrieved therefrom. The memory may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, and/or any other type of volatile or nonvolatile physical or tangible computer-readable media.

In exemplary embodiments, computer-executable instructions may be stored in the memory for execution by a processor to particularly cause the processor to perform one or more of the functions described herein, such that the memory is a physical, tangible, and non-transitory computer readable storage media. Such instructions often improve the efficiencies and/or performance of the processor that is performing one or more of the various operations herein. It should be appreciated that the memory may include a variety of different memories, each implemented in one or more of the functions or processes described herein.

In exemplary embodiments, a network interface may be coupled to (and in communication with) the processor and the memory. The network interface may include, without limitation, a wired network adapter, a wireless network adapter, a mobile network adapter, or other device capable of communicating to one or more different networks. In some exemplary embodiments, one or more network interfaces may be incorporated into or with the processor.

It should be appreciated that the functions described herein, in some embodiments, may be described in computer executable instructions stored on a computer readable media, and executable by one or more processors. The computer readable media is a non-transitory computer readable storage medium. By way of example, and not limitation, such computer-readable media can include RAM, ROM, EEPROM, CD-ROM or other optical disk storage, magnetic disk storage or other magnetic storage devices, or any other medium that can be used to carry or store desired program code in the form of instructions or databases and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

Example embodiments are provided so that the present disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the present disclosure. In some example embodiments, well-known processes, well-known device structures, and well-known technologies are not described in detail. For example, technical material that is known in the technical fields related to the present disclosure has not been described in detail so that the present disclosure is not unnecessarily obscured. This includes, but is not limited, to technology utilized in determining the location of mobile devices via a variety of means. In addition, advantages and improvements that may be achieved with one or more exemplary embodiments of the present disclosure are provided for purposes of illustration only and do not limit the scope of the present disclosure, as exemplary embodiments disclosed herein may provide all or none of the above mentioned advantages and improvements and still fall within the scope of the present disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. In addition, as used herein, the term “or” is an inclusive “or” operator, and is equivalent to the term “and/or,” unless the context clearly dictates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, or features, these elements, components, or features should not be limited by these terms. These terms may be only used to distinguish one element, component, or feature from another element, component, or feature. Terms such as “first,” “second,” and other numerical terms when used herein do not imply a sequence or order unless clearly indicated by the context. Thus, a first element, component, or feature could be termed a second element, component, or feature without departing from the teachings of the example embodiments.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

The foregoing description of the embodiments has been provided for purposes of illustration and description. It is not intended to be exhaustive or to limit the present disclosure. Individual elements, intended or stated uses, or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the present disclosure, and all such modifications are intended to be included within the scope of the present disclosure.

What is claimed is:

1. A system for managing specialty community agriculture and associated resources utilizing needs-based, context-based, and behavior-driven integrated production and consumption resource management capabilities and resource inputs, the system comprising a plurality of different devices, sensors, other systems, and/or communications network(s) configured to dynamically and flexibly manage modular planting, growing, treatment, harvesting, producing, acquiring, generating, distribution, storage, and/or consumption of consumable resources of a community(s), wherein:
the system is configured to be operable for determining, assessing, analyzing, (re)allocating, and/or predicting future production and/or consumption resources to support future forecasted, predicted, and/or possible needs and associated behaviors and contexts of at least a plurality of humans within, expected to be within, and/or requirement to be within a human community population of the community(s).

2. The system of claim 1, wherein the system is configured for utilizing a plurality of digital agents that measure, monitor, track, assess and/or analyze human(s), equipment, and/or community(s) needs, contexts, behaviors, and associated resources, to develop, proffer, and/or implement recommendations associated with said needs, contexts, behaviors and/or associated resources using a scoring capability(s).

3. The system of claim 2, wherein the associated resources include one or more agricultural inputs and outputs, life support, energy, water, soil and/or regolith, structural and construction, communication, transportation, healthcare, environmental control, scientific and research, security and safety, social and recreational, supply chain and logistics, crew and support, and/or sustainable, physical, electronic, mental health, and/or intangible resources needed to meet said needs.

4. The system of claim 2, wherein:
the needs-based capabilities include assessment of past, present, and future production and/or consumption needs and associated resources;
the needs include needs of an entity comprising two or more of Physiological, Safety, Love/Relationships, Esteem, Actualization, Transcendence, setup, configuration, operating, maintenance, and/or retirement needs, with such needs being associated with one or more resources; and
the needs of a community include two or more of physical, mental health, stress, nutritional, occupational and skills, family, age and life stage, education and skill development, age and life stage, energy and water usage, financial, community engagement, environmental impact, emergency preparedness, transportation, cultural and social preferences, and resource feedback and reporting capabilities.

5. The system of claim 2, wherein the contexts include two or more of social, work, cultural, economic, family, health, environmental, political, technology, emergency or crisis, legal, religious or spiritual, psychological, and/or recreational contexts.

6. The system of claim 2, wherein the behaviors include one or more of prosocial, antisocial, altruistic, aggressive, cooperative, competitive, assertive passive, impulsive, risk-taking, procrastination, adaptive, maladaptive, empathetic, conformity, nonconformity, risk-adverse, risk-seeking, self-regulation, and/or help-seeking behaviors.

7. The system of claim 2, wherein the system is configured such that the needs, contexts, behaviors, and associated resources are capable of being measured, determined, estimated, monitored, and/or tracked, with such capability(s) being able to determine, estimate, and/or generate a score or an input into a scoring system(s).

8. The system of claim 7, wherein the system is configured such that the scoring system(s) utilizes one or more of a quantitative, numerical qualitative, color coding, binary, ordinal ranking, textual feedback, graphical representation(s), heatmap(s), percentage-based, checklist, threshold-based, range-based, risk matrices, machine learning/AI, natural language processing, sensor alarms, health-based and/or behavioral analysis algorithm.

9. The system of claim 7, wherein the system is configured such that score(s) of the scoring system(s) serve as inputs and/or mechanisms into recommendation development and implementation of changes/modifications to needs, behaviors, contexts, and/or resource utilization, resource (re)allocation and associated processes, and/or dynamically and flexibly manage modular planting, growing, treatment, harvesting, producing, acquiring, generating, distribution, storage, and/or consumption of consumable resources of the community(s).

10. The system of claim 9, wherein the system is configured such that behavior recommendations are based on the context associated with behavior, an entity and/or community needs that need to be addressed by the individual, and on the potential for positive impact to the entity's personal and/or community score.

11. The system of claim 1, wherein the system is configured to be operable for prescribing and/or applying artificial electromagnetic waves and/or fields to help support plant growth and health and/or to help support health, safety, and/or treatment of at least one entity within the community.

12. The system of claim 11, wherein:
the community is an off-Earth community; and
the system is configured to be operable for prescribing and/or applying artificial electromagnetic waves and/or fields to help support plant growth and health and/or to help support health, safety, and/or treatment for the at least one entity within the off-Earth community to preempt and/or lower the risk of a future occurrence of a psychological episode, which may be linked to reduced protection of the Earth's electromagnetic field coupled with a solar event(s).

13. The system of claim 1, wherein the plurality of different devices, sensors, other systems, and/or communications network(s) comprises at least one digital agent configured to be operable for developing, proffering, and/or implementing recommendations for altering behavior of at least one entity of the community which altered behavior will benefit the community in terms of land management, environment, and/or climate.

14. The system of claim 1, wherein the system is configured to be operable for developing, proffering, and/or implementing recommendations regarding seeds including saving sprouts and recovery along with breeding and genetic modification non-earth gravity.

15. The system of claim 1, wherein the system is configured to determine, through a plurality of measurements/readings taken by the plurality of different devices, sensors, other systems, and/or communication network(s) and/or through information from and/or about system inputs, behavior(s) of at least one entity of the community and:
(a) context(s) associated with the behavior(s) of the at least one entity; or
(b) location and the context(s) associated with the behavior(s) of the at least one entity.

16. The system of claim 15, wherein the at least one entity comprises one or more of a human, an animal, a plant, another system, a machine, a robot, an artificial intelligence, a virtual agent, a corporation, a business entity, a nation, a network, a driverless vehicle, a connected vehicle, a drone, and/or a governmental entity.

17. The system of claim 15, wherein the system is configured to:
assess, evaluate, and predict a risk of a future occurrence(s) of context(s) associated with behavior(s) by the at least one entity; and
facilitate one or more actions and/or activities to preempt and/or lower the risk of a future occurrence(s) of context(s) associated with behavior(s) by the at least one entity before the context(s) associated with the behavior(s) occurs when the behavior(s) by the at least one entity is determined to be detrimental to the community.

18. The system of claim 15, wherein:
the system is further configured to dynamically and adaptively determine a reward for incentivizing context(s) associated with behavior(s) of the at least one entity that is beneficial to the community; and/or
the system is further configured to dynamically and adaptively determine a disincentive for disincentivizing context(s) associated with behavior(s) of the at least one entity that is detrimental to the community.

19. The system of claim 15, wherein:
the system is configured to capture the behavior(s) and the context(s) of the least one entity in a system of record for tracking, managing, and redeeming reward(s) and disincentive(s);
the behavior(s) of the at least one entity includes a contribution of the at least one entity to the community as it relates to the planting, growing, treatment, harvesting, producing, acquiring, generating, distribution, storage, and/or consumption of consumable resources of the community;
the system is configured to quantitatively and qualitatively assess the contribution of the at least one entity to the community and assign contribution assessment, score, level, or metric(s) where the contribution of the at least one entity is measured against one or more participation and/or performance targets after the at least one entity's participation and/or performance has been adjusted to incorporate contextual situations, issues, and/or other factors resulting in a context outside expected parameters that may have impacted the at least one entity's participation and/or performance; and
the system is configured to assign, award, and/or allocate an electronic or physical store of value for the contribution of the at least one entity to the community, which may be based in part on a comparison of the contribution assessment, score, level, or metric(s) assigned to the contribution made by the at least one entity with the contribution assessments, scores, levels, or metrics assigned to contributions made by other entities of the community.

20. The system of claim 19, wherein:
the system of record is a ledger, distributed ledger or blockchain system; and/or
the electronic or physical store of value comprises one or more of money, a ticket, cryptocurrency, and a credit for a certain amount/type of consumable; and/or
the electronic or physical store of value is transferrable from one entity to another entity; and/or
the electronic or physical store of value is redeemable for cybercurrency and/or transferrable to an electronic wallet.

21. The system of claim 19, wherein the at least one entity's participation and/or performance is measured against one or more participation and/or performance targets for a specialty farming and/or agricultural production process as a whole or for a combination of less than all of the steps, parts, and/or sub-processes of the specialty farming and/or agricultural production process.

22. The system of claim 19, wherein:
the system is configured to capture to every contribution made by entities to the community relating to the planting, growing, treatment, harvesting, producing, acquiring, generating, distribution, storage, and/or consumption of consumable resources of the community; and
the system is configured to quantitatively and qualitatively value the contributions made by the entities to the community where the contributions are measured against one or more participation and/or performance targets once the participation and/or performance has been adjusted to incorporate contextual situations, issues, and/or other factors resulting in a context outside expected parameters that may have impacted the participation and/or performance; and
the system is configured to assign, award, and/or allocate electronic or physical stores of value for the contributions made by the entities to the community, which may be based in part on a comparison of the contribution assessments, scores, levels, or metrics assigned to the contributions made by the entities of the community.

23. The system of claim 22, wherein the electronic or physical stores of value assigned, awarded, and/or allocated by the system for the contributions made by the entities to the community comprise credits for certain amounts/types of consumables, thereby allowing for merit-based allocation of the consumables to the entities of the community, whereby the credits may be tied to financial cost of the consumable, social impact, nutritional requirements individually or broadly, or other variables(s).

24. The system of claim 19, wherein:
the system is configured to be operable for identifying a root cause(s) of a given negative measure and for qualifying, reducing an impact of, and/or otherwise nullifying a negative metric when the root cause(s) demonstrates that the root cause(s) was out of the control of the at least one entity; and/or
the system is configured to quantitatively and qualitatively assess contributions made by entity(ies) to the community and assign contribution assessment, score, level, or metric(s) based on fixed objective criteria and criteria relative to other entity(ies) including other persons, robots, and/or assets of the community.

25. The system of claim 19, wherein:
the system is configured to capture to contributions made by entities to the community relating to the planting, growing, treatment, harvesting, producing, acquiring, generating, distribution, storage, and/or consumption of consumable resources of the community; and
the system is configured to quantitatively and qualitatively value or score the contributions made by the entities to the community for helping to determine allocation of the community's resources.

26. The system of claim 1, wherein the plurality of different devices, sensors, other systems, and/or communications network(s) comprises at least one digital agent associated with at least one entity of the community.

27. The system of claim 26, wherein:
the system is configured to search for, detect, measure, monitor, track, control, manage, react, and/or respond to one or more triggers of a human asset of the community by utilizing the at least one digital agent;
the digital agent is configured with specialized machine learning/AI-based capability(ies) to focus on searching for, detection, measuring, monitoring, tracking, controlling, managing, reacting, and/or responding to a particular trigger and its related triggers by utilizing data sets associated with the searching for, detection, measuring, monitoring, tracking, controlling, managing, reacting, and/or responding to the one or more triggers and their related triggers by utilizing and/or combining with data associated with the human's historical interaction of and/or association with said one or more triggers and their related triggers, including occurrences of the one or more triggers and their related triggers, the behaviors and contexts preceding, causing, associated with, or resulting from the occurrence of the one or more triggers and their related triggers;
the contexts include one or more of where, when, why, who, what, and/or how associated with the behavior(s) preceding, causing, being associated with, and/or resulting from the occurrence of the one or more triggers and their related triggers; and
the one or more triggers and their related triggers are associated with mental and/or physical behavior and/or situational ranges, levels, break points, targets, or milestones that, once reached or increasing in risk of being reached and/or occurring, can materially increase the potential for changes in behavior that can cause negative or positive results associated with that change in behavior.

28. The system of claim 26, wherein:
the digital agent is local to the at least one entity including embedded, attached, remote, and/or cloud based that communicates with surroundings using a variety of sensors and/or interfaces; and/or
the digital agent comprises one or more specialty trigger agents configured to interact with one or more other specialty functional agents, such as for location and context; and/or
the digital agent comprises one or more specialized agents configured to focus on location and context detection, historical tracking, future location and/or context prediction and action generation; and/or
the digital agent is configured to predict future location based on historical and current location and associated contexts for both the at least one entity individually as well as other entities that have in the past encountered similar location/contexts;
and/or the digital agent comprises an artificial intelligent (AI) assistant-type bot, assistant, aid, or agent; and/or
the digital agent comprises an intelligent agent (IA) configured to perceive its environment, to take action(s) autonomously in order to achieve a goal(s), and to improve its performance with learning and/or knowledge; and/or
the digital agent is configured to perceive its environment through the plurality of different devices, sensors, other systems, and/or communications network(s) and to act upon that environment through one or more actuators.

29. The system of claim 26, wherein the digital agent comprises one or more of a specialty agent or bot dedicated to one or more particular functions and/or data specialty areas, a Medical Bot specialized within a medical field(s), a cancer diagnostic bot, a sepsis treatment bot, a location analysis and prediction bot (Chat GPS), a forensics bot, a context determination bot, a trigger detection bot, a behavior determination bot, a mental state bot, and/or a digital agent comprising a natural language processing tool driven by AI technology.

30. The system of claim 26, wherein the digital agent comprises a bot configured to focus on triggers, trigger detection, and alternative actions based on trigger and context.

31. The system of claim 1, wherein the system is configured to:
determine, through a plurality of measurements/readings taken by the plurality of different devices, sensors, other systems, and/or communication network(s) and/or through information from and/or about system inputs, contexts, and current available and utilized resources of a community, and current and future needs of a community, possible additions, changes, maintenance, and/or removals of one or more resources to and/or from the community; and
cross reference and assess the one or more current resources of the community with the current and future needs of the community to determine whether the community should expand, maintain, and/or reduce one or more resource production;
whereby the system is configured to direct, advise, or otherwise instruct the community to expand, maintain, and/or reduce/remove productive capabilities when the system determines that the community should expand, maintain, and/or reduce a resource(s) production required to support said future needs of the resource(s).

32. The system of claim 31, wherein:
said production utilizes the conversion and/or transformation of regolith into a usable soil state(s) utilizing a plurality of physical weathering, chemical weathering, biological weathering, worm deployment, microbe application, biological process, water/hydration application, pioneer species utilization, algae growth, and/or fungi-based, synthetic, and/or gene engineered organism introduction(s), action(s), method(s), technique(s), mechanism(s), and/or mode(s) of processing; and wherein such conversion, transformation, and/or utilization introduction(s), action(s), method(s), technique(s), mechanism(s), and/or modes of processing are conducted in one or more contexts enabling acceleration in time and/or quantity and/or scale of such conversion and/or transformation of the regolith and/or utilization of the resulting soil(s); and/or
one or more production contexts include one or more natural, artificial, and/or constructed structures, systems, machinery, mechanisms, and/or environments with systemic ability to control one or more aspects of moisture, water interaction, humidity, radiation, light, nitrogen, oxygen, CO2, mineral content, heavy metals, PH, nutrient content, particle size, particle distribution, particle composition, organic matter, microbiome, fungi, aggregation/structure, cation exchange and capacity; and/or
needs include physical, mental, nutritional, hydration-related needs used in and for human consumption, at various levels including necessary for survival for one or more humans, maintenance of status quo, ability to support various population levels and demographics, individual survival and/or physical and mental well-being; and/or
resources include raw materials, regolith, minerals, nutritional-related, light-related, water-based, fertilizers, worms, chemical agents, pharmaceutical agents, microbes, pioneer species, algae, fungis, synthetic organisms, gen-engineered organisms, labor, capital, space, capacity, processing, storage, and transportation capabilities, and interim and end products and/or growth and/or production resulting from use of one or more resources.

33. The system of claim 31, wherein:
the community is an off-Earth community;
the system is configured to be operable for managing agriculture of the off-Earth community;
when the system determines that the off-Earth community should expand resource production, the system is configured to direct, advise, or otherwise instruct the community regarding:
In-Situ Resource Utilization (ISRU);
transformation of local resources into supporting materials for production of bioproducts and support of community health and longevity;
transformation of local resources using ISRU methods including transformation of off-Earth regolith material into locally derived soils to support IRSU based agriculture for the community; and/or
transformation of regolith material including one or more biological(s), chemical alteration, physical alteration, another method of alteration, and combinations thereof.

34. The system of claim 33, wherein the system is configured to:
monitor, through a plurality of measurements/readings taken by the plurality of different devices, sensors, other systems, and/or communication network(s) and/or through information from and/or about system inputs, health of worms that are used to facilitate transformation of off-Earth regolith material into locally derived soils to support IRSU based agriculture for the community; and
determine and facilitate one or more actions, if any, for improving health of the worms.

35. The system of claim of claim 31, wherein the system is configured to provide guidance or direct expansion of productive capacity using data points including context provided by one or more of an IoT device(s), sensor(s), health data, predictive modeling, biomarker(s) such as for pH or water retention, soil cation exchange, particle size or distribution, or other method.

36. The system of claim of claim 31, wherein during expansion of productive capabilities of ISRU soils, the system is configured to account for the expanding capacity in a larger model to support the community and anticipate and suggest best use(s) of the new expanded capacity including one or more of growing staple crops, expanding to specialty goods, more advanced bioproducts (such as supporting biopolymer creation) and/or creating a greenspace for psychological health and well-being.

37. The system of claim 1, wherein the context includes a physical and/or virtual geofence or boundary area, a contextual geofence or boundary area, and/or restrictions associated with that geofence(s) or boundary area(s), and/or restriction(s) associated with the geofence(s) or boundary area(s).

38. The system of claim 37, wherein the geofence or boundary area includes a buffer zone with a varying physical or virtual length, width, and/or height.

39. The system of claim 1, wherein the community comprises a neighborhood that is crime-ridden and/or subject to a turf war between rival gangs, and wherein the system is configured to:
dynamically and adaptively determine a reward for incentivizing behavior for an associated context that increases a likelihood of a future occurrence(s) of a respectful behavior(s) by the at least one entity that is respectful of at least one other entity of the community before the respectful behavior(s) occurs; and/or
dynamically and adaptively determine a disincentive for disincentivizing behavior for an associated context that decreases the likelihood of a future occurrence(s) of a disrespectful behavior(s) by the at least one entity that is disrespectful of at least one other entity of the community before the disrespectful behavior(s) occurs.

40. The system of claim 1, wherein:
the system is configured to dynamically and adaptively determine a reward for incentivizing behavior for an associated context that increases a likelihood of a future occurrence(s) of a respectful behavior(s) by the at least one entity that is respectful of at least one other entity of the community before the respectful behavior(s) occurs and facilitate redemption of the reward including one or more of a material reward, a physical reward, a financial reward, a monetary reward, an electronic reward, a virtual reward, a non-material reward, and a non-financial reward; and/or
the system is configured to dynamically and adaptively determine a disincentive for disincentivizing behavior for an associated context that decreases the likelihood of a future occurrence(s) of a disrespectful behavior(s) by the at least one entity that is disrespectful of at least one other entity of the community before the disrespectful behavior(s) occurs and facilitate redemption of the disincentive including one or more of a material punishment or penalty, a physical punishment or penalty, a financial punishment or penalty, a monetary punishment or penalty, an electronic punishment or penalty, a virtual punishment or penalty, a non-material punishment or penalty, and a non-financial punishment or penalty.

41. The system of claim 1, wherein:
consumables of the community include one or more of food, liquids, minerals, and environmental needs of living organisms in the community needed to sustain existence and health; and/or
the system is configured such that the consumables are organized virtually and/or physically into modules which are based on one or more of: physical attributes, sustenance impact, ability-to-produce conditions and contexts, living entity(ies) consuming the consumable, geographical location, raw material inputs and/or partial or finished good output storage requirements, tools and materials needed for production, growing life cycle, growing temporal requirements, shelf-life duration, expiration expectations, and/or hybrids and combinations thereof.

42. The system of claim 1, wherein:
the system is configured such that consumables of the community are organized virtually and/or physically into modules which include one or more units of seed(s), plant(s), animal(s), mineral(s), liquid(s), and/or gas(es) that can directly or when combined with other produce and/or result in one or more unit(s) of consumable items; and/or
the system is configured to be operable for managing the community by utilizing needs-based production and consumption capabilities that include:
consumables needed to sustain the existence and health of an individual living entity, group(s) or pools of individual living entities, and/or the community of living entities as a whole based on their nutritional needs, living/working environments and contexts, physical and/or mental well-being, ability to interact with other members of the community, and/or ability to contribute to the community in balance with resources needed for support; and/or
condition(s) with, within, or associated with one or more living entity(s), module(s) of production and/or consumption, or related input, in-process, or output of production and/or consumption experience, including situations, circumstances, environments, and states of persons, places, and things.

43. The system of claim 1, wherein the system is configured to be operable for:
monitoring, via the plurality of different devices, sensors, other systems, and/or communications network(s), every entity's interaction with every other entity of the community to determine the interaction impact on one or more community triggers including one or more of anxiety, depression, conflict, and/or motivation level; and
weighing or scoring the monitored interactions, which may include likelihood of a future occurrence, associated criticality for the community, and whether an identified root cause(s) of a given negative measure demonstrates that the root cause(s) was outside or at least partially outside of the entity's control.

44. The system of claim 1, wherein:
the system is configured to be operable for detecting trigger(s), diagnosing root cause(s) of the trigger(s), and developing resource-optimized remedies/actions at the multi-entry level for the community; and/or
the system is configured to influence connected actions between entities of the community such that the outcome of said actions facilitates the achievement of a common goal, objective, motivation, or purpose for the community; and/or
the system is configured to facilitate, control, and/or manage interaction between entities of the community by utilizing a plurality of measurements/readings taken by the plurality of different devices, sensors, other systems, and/or communications network(s); and/or
the system is configured to dynamically redeploy measurement capabilities of the plurality of different devices, sensors, other systems, and/or communications network(s) automatically without manual human intervention or with little human manual intervention.

45. The system of claim 1, wherein the system is configured to calibrate measurement capabilities and results of the plurality of different devices, sensors, other systems, and/or communications network(s) automatically without manual human intervention or with little human manual intervention to accommodate for different context(s) of the community under which the plurality of different devices, sensors, other systems, and/or communications network(s) are being used, whereby the calibration provides the ability to compare data obtained for the community via the plurality of different devices, sensors, other systems, and/or communications network(s) with data obtained elsewhere under different contexts.

46. The system of claim 1, wherein the system is configured to implement a digital twin(s) for an individual(s) of the community that seeks to minor behavior and associated contributions of the individual to the community.

47. The system of claim 1, wherein the system is configured with and/or includes a project and work management system operable for harmonizing or providing harmony between human and robotic entities of the community, the project and work management system including one or more digital agents associated with and/or working on behalf of one or more humans of the community, and one or more digital agents associated with and/or working in coordination with one or more robots of the community, whereby the digital agents are operable for coordinating human/robot behaviors for given tasks in given contexts.

48. The system of claim 1, wherein the system is configured for administering and managing a community's prison-less criminal, civil, and other legal/judicial sentence(s) and/or verdicts and associated punishments associated with an infraction(s) and a violator(s) utilizing community(s), victim(s), and violator(s) needs-based, context-based, and behavior-driven contribution and feedback capabilities and associated punishment and community contribution scoring management capabilities, wherein the plurality of different devices, sensors, other systems, and/or communications network(s) configured to dynamically and flexibly manage the needs, behaviors, contexts, triggers, and resource utilization/usage associated with the infractions and/or associated punishment to satisfy, reduce, and/or modify said punishments and/or pre-empt, prevent, and/or mitigate current and/or future needs, behaviors, triggers, and/or resource utilization/usage, and associated contexts, and pre-empt, identify, proffer, recommend, implement, and/or facilitate additions, modifications, and/or deletions to a violator's needs, behaviors, triggers, and/or resource utilization/usage and associated contexts that will satisfy, reduce, and/or modify said punishments, and/or benefit the community, the victim(s) of the infraction(s), and/or the violator(s) themselves.

49. A system for administering and managing a community's prison-less criminal, civil, and other legal/judicial sentence(s) and/or verdicts and associated punishments associated with an infraction(s) and a violator(s) utilizing community(s), victim(s), and violator(s) needs-based, context-based, and behavior-driven contribution and feedback capabilities and associated punishment and community contribution scoring management capabilities, the system comprising a plurality of different devices, sensors, other systems, and/or communications network(s) configured to dynamically and flexibly manage the needs, behaviors, contexts, triggers, and resource utilization/usage associated with the infractions and/or associated punishment to satisfy, reduce, and/or modify said punishments and/or pre-empt, prevent, and/or mitigate current and/or future needs, behaviors, triggers, and/or resource utilization/usage, and associated contexts, and pre-empt, identify, proffer, recommend, implement, and/or facilitate additions, modifications, and/or deletions to a violator's needs, behaviors, triggers, and/or resource utilization/usage and associated contexts that will satisfy, reduce, and/or modify said punishments, and/or benefit the community, the victim(s) of the infraction(s), and/or the violator(s) themselves.

50. The system of claim 49, wherein the system is configured for utilizing a plurality of digital agents that measure, monitor, track, assess and/or analyze a violator(s), victim(s), and/or community(s) needs, contexts, behaviors, triggers, and/or resource utilization/usage to develop, proffer, facilitate, and/or implement contribution, feedback, and/or punishment reduction recommendations associated with the needs, contexts, behaviors, triggers, and/or resources using a scoring capability(s).

51. The system of claim 50, wherein the system is configured for using the plurality of digital agents in the management of said prison-less system including identifying, recommending, facilitating, and/or otherwise implementing action(s) for the violator(s) to take that will increase the likelihood of a positive impact to the community, victim(s) and/or violator(s) and/or that will decrease the likelihood of a negative impact to the community, victim(s), and/or violator(s).

52. The system of claim 49, wherein the system is configured for using a ledger-based transaction capture system including a ledger, distributed ledger or blockchain system for recording measurements, determinations, estimates, monitoring, and/or tracking of the violator(s), community(s), and/or victim(s) needs, behaviors, triggers, contexts, resource utilization/usage, punishments, recommendations, recommendations adherence and associated values, inputs to a score, and scores/scoring.

53. The system of claim 49, wherein the system is configured such that the contribution and feedback capability(s) and/or associated punishments include one or more of community service, fines and restitution, probation, restorative justice, education and skill-building, electronic monitoring, counseling and rehabilitation, community-based corrections, community panels, and/or mediation and conflict resolution capabilities.

54. The system of claim 49, wherein the system is configured such that the recommendations are associated with a value(s), score(s), and/or input(s) to a score based on its impact on and/or contribution to the community(s), victim(s), and/or violator(s) needs, behavior(s), triggers(s), context(s), and/or resource utilization/usage.

55. The system of claim 49, wherein the system is configured such that the contribution and feedback capability(s), associated punishments, and/or community(s), victim(s), and/or violator(s) need(s), behavior(s), trigger(s), contexts and/or resources utilized/used are associated with a scoring system.

56. The system of claim 49, wherein:
the system is configured such that the contribution and feedback capability(s), associated punishments, and/or community(s), victim(s), and/or violator(s) need(s), behavior(s), trigger(s), contexts and/or resources utilized/used are associated with one or more value(s), score(s), and/or input(s) to a score; and
current or future violator(s), victim(s), and/or community contribution value(s), score(s), and/or input(s) to a score can be estimated based on the violator′ (s) past, current and/or predicted needs, behaviors, triggers, and/or resource utilization/usage, and associated contexts, and used to prioritize, weight, and/or otherwise influence the recommendations, and/or impact the violator(s) punishment(s) by decreasing, lowering, diminishing, reducing, and/or otherwise satisfying the punishment(s), and/or increasing, enhancing, adding to, and/or otherwise increasing in severity the punishment(s); and
a violator's punishment(s) and/or associated value(s), score(s), and/or input(s) to a score can be decreased, lowered, diminished, reduced, and/or otherwise satisfied, and/or increased, enhanced, added to, and/or otherwise increased in severity, depending upon the violator's need(s), behavior(s), trigger(s), and/or resource utilization/usage, and associated contexts, and/or to the degree, timeliness, effectiveness, efficiency, accuracy, and/or level of quality the violator follows, implements, and/or satisfies the system(s) recommendations.

57. The system of claim 49, wherein the system is configured for use with a plurality of violators such that:
a behavior of one violator that has a negative impact to the community, victim(s), and/or violators is detrimental to the other violators; and
a behavior of one violator that has a positive impact to the community, victim(s), and/or violators is beneficial to the other violators.

58. The system of claim 49, wherein the system is configured such that:
punishments, associated monitoring, recommendation development, recommendation adherence, and/or scoring is done in a tiered and/or hierarchal structure; and/or
monitoring and/or estimating/predicting of a violator(s), victim(s), and/or community(s) needs, behaviors, triggers, and/or resource utilization/usage and/or associated contexts and application to a violator's punishment and/or victim(s) and/or community(s) contribution score(s) are done in a tiered and/or hierarchical structure.

59. The system of claim 58, wherein the tiered and/or hierarchical structure is qualitative, quantitative, or a combination thereof.

60. The system of claim 59, wherein the tiered and/or hierarchical structure includes a scoring system(s) that utilizes one or more of a quantitative, numerical qualitative, color coding, binary, ordinal ranking, textual feedback, graphical representation(s), heatmap(s), percentage-based, checklist, threshold-based, range-based, risk matrices, machine learning/AI, natural language processing, sensor alarms, health-based and/or behavioral analysis algorithm.

61. The system of claim 59, wherein the tiered and/or hierarchical structure is configured to be tailored to needs, behaviors, contexts, location, triggers, and/or resource utilization.

62. The system of claim 59, wherein the system is configured such that a recommendation and/or degree of adherence to a recommendation is weighted according to importance, criticality, and/or impact to the violator(s), victim(s), and/or community(s), whereby the weighting depends on the need, behavior, trigger, and/or resource utilization/usage and associated context.

63. The system of claim 49, wherein the system is configured to include a learning and feedback mechanism that enables the system to analyze and modify what is tracked/measured/scored, how the tracking/measurements/scoring is performed, how the tracking/measurements/scoring is performed, how recommendations are developed, and how a punishment/punishment score can be reduced/increased based on the system learning about the violator(s) and/or learning about changes in the needs of the victim(s), the community(s), and/or violator(s).

64. The system of claim 49, wherein the system is configured such that:
the system is configured such that the contribution and feedback capability(s), associated punishments, and/or community(s), victim(s), and/or violator(s) need(s), behavior(s), trigger(s), contexts and/or resources utilized/used are associated with a scoring system;
the system is configured to assign, award, and/or allocate an electronic or physical store of value based on a score(s) of the scoring system; and
the electronic or physical store of value comprises one or more of money, a ticket, cryptocurrency, and a credit for a certain amount/type of consumable; and/or the electronic or physical store of value is transferrable from one entity to another entity;
and/or the electronic or physical store of value is redeemable for cybercurrency and/or transferrable to an electronic wallet.

65. The system of claim 49, wherein the system is configured with the ability for setting punishment(s) based on possible contribution(s) the violator(s) is able to make including contribution(s) made by the violator(s) that have a positive impact on need(s) of the victim(s) and/or community(s) thereby allowing the violator(s) to interactively reduce the punishment(s) and achieve rehabilitation by making the contribution(s) that have a positive impact on need(s) of the victim(s).

66. The system of claim 49, wherein the system is configured such that a violator(s) one of more needs, behaviors, triggers, contexts, and/or resource utilization/usage can be measured, determined, estimated, monitored and/or tracked with such capability(s) being able to determine, estimate, and/or generate a community contribution score or a value associated with a community contribution score(s) for that need(s), behavior(s), trigger(s), context(s) and/or resource usage(s).
